(19) United States
(12) Patent Application Publication
(10) Pub. No.: US 2009/0029996 A1
(43) Pub. Date: Jan. 29, 2009

(54) Pyridazin-3(2H)-One Derivatives And Their Use As Pde4 Inhibitors

(76) Inventors: Nuria Aguilar Izquierdo, Barcelona (ES); Marta Carrascal Riera, Barcelona (ES); Vittorio Dal Piaz, Impruneta (IT); Jordi Gracia Ferrer, Barcelona (ES); Wenceslao Lumeras Amador, Sant Just Desvern (ES); Maria Del Carmen Masdeu Margalef, Barcelona (ES); Graham Warrellow, Hertfordshire (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/629,527

(22) PCT Filed: Jun. 21, 2005
(86) PCT No.: PCT/EP05/06712

§ 371 Date: Feb. 12, 2008

(30) Foreign Application Priority Data
Jun. 21, 2004 (ES) P200401512

(51) Int. Cl. A61K 31/5377; C07D 403/02; A61K 31/501; A61P 19/02; A61P 1/00; A61P 17/00; A61P 11/00; C07D 413/14

(52) U.S. Cl. 514/236.5; 544/238; 514/252.04; 514/252.03; 544/114

(57) ABSTRACT

The invention relates to new therapeutically useful pyridazin-3(2H)-one derivatives, to processes for their preparation and to pharmaceutical compositions containing them. These compounds are potent and selective inhibitors of phosphodiesterase 4 (PDE4) and are thus useful in the treatment, prevention and suppression of related pathological conditions, diseases and disorders, in particular asthma, chronic obstructive pulmonary disease, rheumatoid arthritis, topic dermatitis, psoriasis or irritable bowel disease.

The present invention relates to new therapeutically useful pyridazin-3(2H)-one derivatives, to processes for their preparation and to pharmaceutical compositions containing them. These compounds are potent and selective inhibitors of phosphodiesterase 4 (PDE4) and are thus useful in the treatment, prevention or suppression of pathological conditions, diseases and disorders known to be susceptible of being improved by inhibition of PDE4.

Phosphodiesterases (PDEs) comprise a superfamily of enzymes responsible for the hydrolysis and inactivation of the second messengers cyclic adenosine monophosphate (cAMP) and cyclic guanosine monophosphate (cGMP). Eleven different PDE families have been identified to date (PDE1 to PDE11) which differ in substrate preference, catalytic activity, sensitivity to endogenous activators and inhibitors, and encoding genes.

The PDE4 isoenzyme family exhibits a high affinity for cyclic AMP but has weak affinity for cyclic GMP. Increased cyclic AMP levels caused by PDE4 inhibition are associated with the suppression of cell activation in a wide range of inflammatory and immune cells, including lymphocytes, macrophages, basophils, neutrophils, and eosinophils. Moreover, PDE4 inhibition decreases the release of the cytokine Tumor Necrosis Factor α (TNFα). The biology of PDE4 is described in several recent reviews, for example M. D. Houslay, Prog. Nucleic Acid Res. Mol. Biol. 2001, 69, 249-315; J. E. Souness et al. Immunopharmacol. 2000 47, 127-162; or M. Conti and S. L. Jin, Prog. Nucleic Acid Res. Mol. Biol. 1999, 63, 1-38.

In view of these physiological effects, PDE4 inhibitors of varied chemical structures have been recently disclosed for the treatment or prevention of chronic and acute inflammatory diseases and of other pathological conditions, diseases and disorders known to be susceptible to amelioration by inhibition of PDE4. See, for example, U.S. Pat. No. 5,449,686, U.S. Pat. No. 5,710,170, WO 98/45268, WO 99/06404, WO 01/57025, WO 01/57036, WO 01/46184, WO 97/05105, WO 96/40636, WO03/097613, U.S. Pat. No. 5,786,354, U.S. Pat. No. 5,773,467, U.S. Pat. No. 5,753,666, U.S. Pat. No. 5,728,712, U.S. Pat. No. 5,693,659, U.S. Pat. No. 5,679,696, U.S. Pat. No. 5,596,013, U.S. Pat. No. 5,541,219, U.S. Pat. No. 5,508,300, U.S. Pat. No. 5,502,072 or H. J. Dyke and J. G. Montana, Exp. Opin. Invest. Drugs 1999, 8, 1301-1325.

A few compounds having the capacity to selectively inhibit phosphodiesterase 4 are in active development. Examples of these compounds are cipamfylline, arofyline, cilomilast, roflumilast, mesopram and pumafentrine.

The international applications WO03/097613 A1, WO2004/058729 A1 and WO 2005/049581 describe pyridazin-3(2H)-one derivatives as potent and selective inhibitors of PDE4. We have now found that the compounds of formula (I) described in more detail below have surprising and particularly advantageous properties.

It is known that the clinical development in man of early PDE4 inhibitors such as rolipram has been hampered by the appearance of side effects such as nausea and vomiting at therapeutic plasma levels (Curr. Pharm. Des. 2002, 8, 1255-96). The compounds described in the present invention are potent and selective PDE4 inhibitors which are hydrolized systemically. This particular property provides the compounds with a high local activity and little or no systemic action, avoiding or reducing the risk of unwanted systemic side effects, and makes them useful for the treatment or prevention of these pathological conditions, diseases and disorders, in particular asthma, chronic obstructive pulmonary disease, rheumatoid arthritis, atopic dermatitis, psoriasis or irritable bowel disease.

The compounds of the present invention can also be used in combination with other drugs known to be effective in the treatment of these diseases. For example, they can be used in combination with steroids or immunosuppressive agents, such as cyclosporin A, rapamycin, T-cell receptor blockers, β2-adrenergic agonists or antagonists of M3 muscarinic receptors. In this case the administration of the compounds allows a reduction of the dosage of the other drugs, thus preventing the appearance of the undesired side effects associated with both steroids and immunosuppressants.

Like other PDE4 inhibitors (see references above) the compounds of the invention can also be used for blocking the ulcerogenic effects induced by a variety of etiological agents, such as antiinflammatory drugs (steroidal or non-steroidal antiinflammatory agents), stress, ammonia, ethanol and concentrated acids. They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease.

They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the present invention provides novel compounds of formula (I):

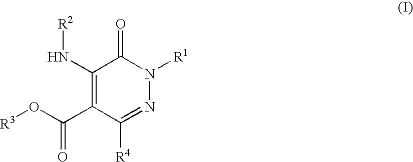

wherein
R¹ represents:
a hydrogen atom;
an alkyl, alkenyl or alkynyl group, which is optionally substituted by one or more substituents selected from halogen atoms and hydroxy, alkoxy, aryloxy, alkylthio, arylthio, oxo, amino, mono- or di-alkylamino, acylamino, hydroxycarbonyl, alkoxycarbonyl, carbamoyl or mono- or di-alkylcarbamoyl groups;
   R² represents a monocyclic or polycyclic heteroaryl group, which is optionally substituted by one or more substituents selected from:
halogen atoms;
alkyl and alkylene groups, which are optionally substituted by one or more substituents selected from halogen atoms and phenyl, hydroxy, alkoxy, aryloxy, alkylthio, arylthio, oxo, amino, mono- or di-alkylamino, acylamino, hydroxycarbonyl, alkoxycarbonyl, carbamoyl or mono- or di-alkylcarbamoyl groups
phenyl, hydroxy, hydroxycarbonyl, hydroxyalkyl, alkoxycarbonyl, alkoxy, cycloalkoxy, nitro, cyano, aryloxy, alkylthio, arylthio, alkylsulfinyl, alkylsulfonyl, alkylsulfamoyl, acyl, amino, mono- or di-alkylamino, acylamino, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono- or di-alkylaminosulfonyl, cyano, difluoromethoxy or trifluoromethoxy groups;
   R³ represents a group of formula:

G-L1-(CRR′)ₙ—

wherein
n is an integer from 0 to 6
R and R′ are independently selected from the group consisting of hydrogen atoms and lower alkyl groups
L1 is a linker selected from the group consisting of a direct bond, —CO—, —NR″—, —NR″—CO—, —O(CO)NR″—, —NR″(CO)O—, —O(CO)—, —O(CO)O—, —(CO)O— and —O(R″O)(PO)O— groups wherein R″ is selected from the group consisting of hydrogen atoms and lower alkyl groups
G is selected from hydrogen atoms and alkyl, alkenyl, alkynyl, cycloalkyl, cycloalkenyl, heterocyclyl, aryl, arylalkyl and heteroaryl groups said groups being optionally substituted with one or more substituents selected from:
halogen atoms;
alkyl and alkenyl groups, which are optionally substituted by one or more substituents selected from halogen atoms; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono- or di-alkylaminosulfonyl, cyano, difluoromethoxy or trifluoromethoxy groups;
   with the proviso that R³ is not a hydrogen atom,
   R⁴ represents a monocyclic or polycyclic aryl or heteroaryl group, which is optionally substituted by one or more substituents selected from:
halogen atoms;
alkyl and alkenyl groups, which are optionally substituted by one or more substituents selected from halogen atoms and phenyl, hydroxy, hydroxyalkyl, alkoxy, aryloxy, alkylthio, arylthio, oxo, amino, mono- or di-alkylamino, acylamino, hydroxycarbonyl, alkoxycarbonyl, carbamoyl, mono- or di-alkylcarbamoyl groups; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono- or di-alkylaminosulfonyl, cyano, difluoromethoxy or trifluoromethoxy groups;
   and the pharmaceutically acceptable salts or N-oxides thereof.

Further objectives of the present invention are to provide processes for preparing said compounds; pharmaceutical compositions comprising an effective amount of said compounds; the use of the compounds in the manufacture of a medicament for the treatment of diseases susceptible of being improved by inhibition of PDE4; and methods of treatment of diseases susceptible to amelioration by inhibition of PDE4, which methods comprise the administration of the compounds of the invention to a subject in need of treatment.

As used herein the term alkyl embraces optionally substituted, linear or branched radicals having 1 to 20 carbon atoms or, preferably 1 to 12 carbon atoms. More preferably alkyl radicals are “lower alkyl” radicals having 1 to 8, preferably 1 to 6 and more preferably 1 to 4 carbon atoms.

Examples include methyl, ethyl, n-propyl, i-propyl, n-butyl, sec-butyl, t-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

As used herein, the term alkenyl embraces optionally substituted, linear or branched, mono or polyunsaturated radicals having 1 to 20 carbon atoms or, preferably, 1 to 12 carbon atoms. More preferably alkenyl radicals are “lower alkenyl” radicals having 2 to 8, preferably 2 to 6 and more preferably 2 to 4 carbon atoms. In particular it is preferred that the alkenyl radicals are mono or diunsaturated.

Examples include vinyl, allyl, 1-propenyl, isopropenyl, 1-butenyl, 2-butenyl, 3-butenyl, 1-pentenyl, 2-pentenyl, 3-pentenyl and 4-pentenyl radicals.

As used herein, the term alkynyl embraces optionally substituted, linear or branched, mono or polyunsaturated radicals having 1 to 20 carbon atoms or, preferably, 1 to 12 carbon atoms. More preferably, alkynyl radicals are “lower alkynyl” radicals having 2 to 8, preferably 2 to 6 and more preferably 2 to 4 carbon atoms. In particular, it is preferred that the alkynyl radicals are mono or diunsaturated.

Examples include 1-propynyl, 2-propynyl, 1-butynyl, 2-butynyl and 3-butynyl radicals.

When it is mentioned that alkyl, alkenyl or alkynyl radicals may be optionally substituted it is meant to include linear or branched alkyl, alkenyl or alkynyl radicals as defined above, which may be unsubstituted or substituted in any position by one or more substituents, for example by 1, 2 or 3 substituents. When two or more substituents are present, each substituent may be the same or different.

A said optionally substituted alkenyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, substituents on an alkenyl group are themselves unsubstituted.

A said optionally substituted alkynyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, substituents on an alkynyl group are themselves unsubstituted.

A said optionally substituted alkyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, substituents on an alkyl group are themselves unsubstituted. Preferred optionally substituted alkyl groups are unsubstituted or substituted with 1, 2 or 3 fluorine atoms.

As used herein, the term alkylene embraces divalent alkyl moieties typically having from 1 to 6, for example from 1 to 4, carbon atoms. Examples of C₁-C₄ alkylene radicals include methylene, ethylene, propylene, butylene, pentylene and hexylene radicals.

A said optionally substituted alkylene group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms.

When an alkylene radical is present as a substituent on another radical it shall be deemed to be a single substituent, rather than a radical formed by two substituents.

As used herein, the term alkoxy (or alkyloxy) embraces optionally substituted, linear or branched oxy-containing radicals each having alkyl portions of 1 to 10 carbon atoms. More preferred alkoxy radicals are “lower alkoxy” radicals having 1 to 8, preferably 1 to 6 and more preferably 1 to 4 carbon atoms.

An alkoxy group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkoxy group are themselves unsubstituted.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy and 2-hydroxypropoxy.

As used herein, the term alkylthio embraces radicals containing an optionally substituted, linear or branched alkyl radicals of 1 to 10 carbon atoms attached to a divalent sulfur atom. More preferred alkylthio radicals are “lower alkylthio” radicals having 1 to 8, preferably 1 to 6 and more preferably 1 to 4 carbon atoms.

An alkylthio group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkylthio group are themselves unsubstituted.

Preferred optionally substituted alkylthio radicals include methylthio, ethylthio, n-propylthio, i-propylthio, n-butylthio, sec-butylthio, t-butylthio, trifluoromethylthio, difluoromethylthio, hydroxymethylthio, 2-hydroxyethylthio and 2-hydroxypropylthio.

As used herein, the term monoalkylamino embraces radicals containing an optionally substituted, linear or branched alkyl radicals of 1 to 10 carbon atoms attached to a divalent —NH— radical. More preferred monoalkylamino radicals are “lower monoalkylamino” radicals having 1 to 8, preferably 1 to 6 and more preferably 1 to 4 carbon atoms.

A monoalkylamino group typically contains an alkyl group which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substitutents on a monoalkylamino group are themselves unsubstituted.

Preferred optionally substituted monoalkylamino radicals include methylamino, ethylamino, n-propylamino, i-propylamino, n-butylamino, sec-butylamino, t-butylamino, trifluoromethylamino, difluoromethylamino, hydroxymethylamino, 2-hydroxyethylamino and 2-hydroxypropylamino.

As used herein, the term dialkylamino embraces radicals containing a trivalent nitrogen atoms with two optionally substituted, linear or branched alkyl radicals of 1 to 10 carbon atoms attached thereto. More preferred dialkylamino radicals are “lower dialkylamino” radicals having 1 to 8, preferably 1 to 6 and more preferably 1 to 4 carbon atoms in each alkyl radical.

A dialkylamino group typically contains two alkyl groups, each of which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylamino group are themselves unsubstituted.

Preferred optionally substituted dialkylamino radicals include dimethylamino, diethylamino, methyl(ethyl)amino, di(n-propyl)amino, n-propyl(methyl)amino, n-propyl(ethyl)amino, di(i-propyl)amino, i-propyl(methyl)amino, i-propyl(ethyl)amino, di(n-butyl)amino, n-butyl(methyl)amino, n-butyl(ethyl)amino, n-butyl(i-propyl)amino, di(sec-butyl)amino, sec-butyl(methyl)amino, sec-butyl(ethyl)amino, sec-butyl(n-propyl)amino, sec-butyl(i-propyl)amino, di(t-butyl)amino, t-butyl(methyl)amino, t-butyl(ethyl)amino, t-butyl(n-propyl)amino, t-butyl(i-propyl)amino, trifluoromethyl(methyl)amino, trifluoromethyl(ethyl)amino, trifluoromethyl(n-propyl)amino, trifluoromethyl(i-propyl)amino, trifluoromethyl(n-butyl)amino, trifluoromethyl(sec-butyl)amino, difluoromethyl(methyl)amino, difluoromethyl(ethyl)amino, difluoromethyl(n-propyl)amino, difluoromethyl(i-propyl)amino, difluoromethyl(n-butyl))amino, difluoromethyl(sec-butyl)amino, difluoromethyl(t-butyl)amino, difluoromethyl(trifluoromethyl)amino, hydroxymethyl(methyl)amino, ethyl(hydroxymethyl)amino, hydroxymethyl(n-propyl)amino, hydroxymethyl(i-propyl)amino, n-butyl(hydroxymethyl)amino, sec-butyl(hydroxymethyl)amino, t-butyl(hydroxymethyl)amino, difluoromethyl(hydroxymethyl)amino, hydroxymethyl(trifluoromethyl)amino, hydroxyethyl(methyl)amino, ethyl(hydroxyethyl)amino, hydroxyethyl(n-propyl)amino, hydroxyethyl(i-propyl)amino, n-butyl(hydroxyethyl)amino, sec-butyl(hydroxyethyl)amino, t-butyl(hydroxyethyl)amino, difluoromethyl(hydroxyethyl)amino, hydroxyethyl(trifluoromethyl)amino, hydroxypropyl(methyl)amino, ethyl(hydroxypropyl)amino, hydroxypropyl(n-propyl)amino, hydroxypropyl(i-propyl)amino, n-butyl(hydroxypropyl)amino, sec-butyl(hydroxypropyl)amino, t-butyl(hydroxypropyl)amino, difluoromethyl(hydroxypropyl)amino, hydroxypropyl(trifluoromethyl)amino.

As used herein, the term hydroxyalkyl embraces linear or branched alkyl radicals having 1 to 10 carbon atoms, preferably 1 to 6 carbon atoms, any one of which may be substituted with one or more hydroxyl radicals.

Examples of such radicals include hydroxymethyl, hydroxyethyl, hydroxypropyl, hydroxybutyl and hydroxyhexyl.

As used herein, the term alkoxycarbonyl embraces optionally substituted, linear or branched radicals each having alkyl portions of 1 to 10 carbon atoms and attached to an oxycarbonyl radical. More preferred alkoxycarbonyl radicals are “lower alkoxycarbonyl” radicals, in which the alkyl moiety has 1 to 8, preferably 1 to 6 and more preferably 1 to 4 carbon atoms.

An alkoxycarbonyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkoxycarbonyl group are themselves unsubstituted.

Preferred optionally substituted alkoxycarbonyl radicals include methoxycarbonyl, ethoxycarbonyl, n-propoxycarbonyl, i-propoxycarbonyl, n-butoxycarbonyl, sec-butoxycarbonyl, t-butoxycarbonyl, trifluoromethoxycarbonyl, difluoromethoxycarbonyl, hydroxymethoxycarbonyl, 2-hydroxyethoxycarbonyl and 2-hydroxypropoxycarbonyl.

As used herein, the term monoalkylcarbamoyl embraces radicals containing an optionally substituted, linear or branched alkyl radicals of 1 to 10 carbon atoms and attached to the nitrogen of a-NHCO— radical. More preferred monoalkylcarbamoyl radicals are “lower monoalkylcarbamoyl” radicals in which the alkyl moiety has 1 to 8, preferably 1 to 6 and more preferably 1 to 4 carbon atoms.

A monoalkylcarbamoyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a monoalkylcarbamoyl group are themselves unsubstituted.

Preferred optionally substituted monoalkylcarbamoyl radicals include methylcarbamoyl, ethylcarbamoyl, n-propylcarbamoyl, i-propylcarbamoyl, n-butylcarbamoyl, sec-butylcarbamoyl, t-butylcarbamoyl, trifluoromethylcarbamoyl, difluoromethylcarbamoyl, hydroxymethylcarbamoyl, 2-hydroxyethylcarbamoyl and 2-hydroxypropylcarbamoyl.

As used herein, the term dialkylcarbamoyl embraces radicals containing a radical NCO— where the nitrogen is attached to two optionally substituted, linear or branched alkyl radicals of 1 to 10 carbon atoms. More preferred dialkylcarbamoyl radicals are “lower dialkylcarbamoyl” radicals having 1 to 8, preferably 1 to 6 and more preferably 1 to 4 carbon atoms in each alkyl radical.

A dialkylcarbamoyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylcarbamoyl group are themselves unsubstituted.

Preferred optionally substituted dialkylcarbamoyl radicals include dimethylcarbamoyl, diethylcarbamoyl, methyl(ethyl)carbamoyl, di(n-propyl)carbamoyl, n-propyl(methyl)carbamoyl, n-propyl(ethyl)carbamoyl, di(i-propyl)carbamoyl, i-propyl(methyl)carbamoyl, i-propyl(ethyl)carbamoyl, di(n-butyl)carbamoyl, n-butyl(methyl)carbamoyl, n-butyl(ethyl)carbamoyl, n-butyl(i-propyl)carbamoyl, di(sec-butyl)carbamoyl, sec-butyl(methyl)carbamoyl, sec-butyl(ethyl)carbamoyl, sec-butyl(n-propyl)carbamoyl, sec-butyl(i-propyl)carbamoyl, di(t-butyl)carbamoyl, t-butyl(methyl)carbamoyl, t-butyl(ethyl)carbamoyl, t-butyl(n-propyl)carbamoyl, t-butyl(i-propyl)carbamoyl, trifluoromethyl(methyl)carbamoyl, trifluoromethyl(ethyl)carbamoyl, trifluoromethyl(n-propyl)carbamoyl, trifluoromethyl(i-propyl)carbamoyl, trifluoromethyl(n-butyl)carbamoyl, trifluoromethyl(sec-butyl)carbamoyl, difluoromethyl(methyl)carbamoyl, difluoromethyl(ethyl)carbamoyl, difluoromethyl(n-propyl)carbamoyl, difluoromethyl(i-propyl)carbamoyl, difluoromethyl(n-butyl))carbamoyl, difluoromethyl(sec-butyl)carbamoyl, difluoromethyl(t-butyl)carbamoyl, difluoromethyl(trifluoromethyl)carbamoyl, hydroxymethyl(methyl)carbamoyl, ethyl(hydroxymethyl)carbamoyl, hydroxymethyl(n-propyl)carbamoyl, hydroxymethyl(i-propyl)carbamoyl, n-butyl(hydroxymethyl)carbamoyl, sec-butyl(hydroxymethyl)carbamoyl, t-butyl(hydroxymethyl)carbamoyl, difluoromethyl(hydroxymethyl)carbamoyl, hydroxymethyl(trifluoromethyl)carbamoyl, hydroxyethyl(methyl)carbamoyl, ethyl(hydroxyethyl)carbamoyl, hydroxyethyl(n-propyl)carbamoyl, hydroxyethyl(i-propyl)carbamoyl, n-butyl(hydroxyethyl)carbamoyl, sec-butyl(hydroxyethyl)carbamoyl, t-butyl(hydroxyethyl)carbamoyl, difluoromethyl(hydroxyethyl)carbamoyl, hydroxyethyl(trifluoromethyl)carbamoyl, hydroxypropyl(methyl)carbamoyl, ethyl(hydroxypropyl)carbamoyl, hydroxypropyl(n-propyl)carbamoyl, hydroxypropyl(i-propyl)carbamoyl, n-butyl(hydroxypropyl)carbamoyl, sec-butyl(hydroxypropyl)carbamoyl, t-butyl(hydroxypropyl)carbamoyl, difluoromethyl(hydroxypropyl)carbamoyl, hydroxypropyl(trifluoromethyl)carbamoyl.

As used herein, the term alkylsulfinyl embraces radicals containing an optionally substituted, linear or branched alkyl radicals of 1 to 10 carbon atoms attached to a divalent —SO— radical. More preferred alkylsulfinyl radicals are “lower alkylsulfinyl” radicals having 1 to 8, preferably 1 to 6 and more preferably 1 to 4 carbon atoms.

An alkylsulfinyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a alkylsulfinyl group are themselves unsubstituted.

Preferred optionally substituted alkylsulfinyl radicals include methylsulfinyl, ethylsulfinyl, n-propylsulfinyl, i-propylsulfinyl, n-butylsulfinyl, sec-butylsulfinyl, t-butylsulfinyl, trifluoromethylsulfinyl, difluoromethylsulfinyl, hydroxymethylsulfinyl, 2-hydroxyethylsulfinyl and 2-hydroxypropylsulfinyl.

As used herein, the term alkylsulfonyl embraces radicals containing an optionally substituted, linear or branched alkyl radicals of 1 to 10 carbon atoms attached to a divalent —SO₂— radical. More preferred alkylsulfonyl radicals are “lower alkylsulfonyl” radicals having 1 to 8, preferably 1 to 6 and more preferably 1 to 4 carbon atoms.

An alkylsulfonyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a monoalkylaminosulfonyl group are themselves unsubstituted.

As used herein, the term monoalkylaminosulfonyl embraces radicals containing an optionally substituted, linear or branched alkyl radicals of 1 to 10 carbon atoms and attached to the nitrogen of a-NHSO₂— radical. More preferred monoalkylaminosulfonyl radicals are “lower monoalkylaminosulfonyl” radicals having 1 to 8, preferably 1 to 6 and more preferably 1 to 4 carbon atoms.

A monoalkylaminosulfonyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a monoalkylaminosulfonyl group are themselves unsubstituted.

Preferred optionally substituted monoalkylaminosulfonyl radicals include methylaminosulfonyl, ethylaminosulfonyl, n-propylaminosulfonyl, i-propylaminosulfonyl, n-butylaminosulfonyl, sec-butylaminosulfonyl, t-butylaminosulfonyl, trifluoromethylaminosulfonyl, difluoromethylaminosulfonyl, hydroxymethylaminosulfonyl, 2-hydroxyethylaminosulfonyl and 2-hydroxypropylaminosulfonyl.

As used herein, the term dialkylaminosulfonyl embraces radicals containing a radical NSO₂— where the nitrogen is attached to two optionally substituted, linear or branched alkyl radicals of 1 to 10 carbon atoms. More preferred dialkylaminosulfonyl radicals are “lower dialkylaminosulfonyl” radicals having 1 to 8, preferably 1 to 6 and more preferably 1 to 4 carbon atoms in each alkyl radical.

A dialkylaminosulfonyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylaminosulfonyl group are themselves unsubstituted.

Preferred optionally substituted dialkylaminosulfonyl radicals include dimethylaminosulfonyl, diethylaminosulfonyl, methyl(ethyl)aminosulfonyl, di(n-propyl)aminosulfonyl, n-propyl(methyl)aminosulfonyl, n-propyl(ethyl)aminosulfonyl, di(i-propyl)aminosulfonyl, i-propyl(methyl)aminosulfonyl, i-propyl(ethyl)aminosulfonyl, di(n-butyl)aminosulfonyl, n-butyl(methyl)aminosulfonyl, n-butyl(ethyl)aminosulfonyl, n-butyl(i-propyl)aminosulfonyl, di(sec-butyl)aminosulfonyl, sec-butyl(methyl)aminosulfonyl, sec-butyl(ethyl)aminosulfonyl, sec-butyl(n-propyl)aminosulfonyl, sec-butyl(i-propyl)aminosulfonyl, di(t-butyl)aminosulfonyl, t-butyl(methyl)aminosulfonyl, t-butyl(ethyl)aminosulfonyl, t-butyl(n-propyl)aminosulfonyl, t-butyl(i-propyl)aminosulfonyl, trifluoromethyl(methyl)aminosulfonyl, trifluoromethyl(ethyl)aminosulfonyl, trifluoromethyl(n-propyl)aminosulfonyl, trifluoromethyl(i-propyl)aminosulfonyl, trifluoromethyl(n-butyl)aminosulfonyl, trifluoromethyl(sec-butyl)aminosulfonyl, difluoromethyl(methyl)aminosulfonyl, difluoromethyl(ethyl)aminosulfonyl, difluoromethyl(n-propyl)aminosulfonyl, difluoromethyl(i-propyl)aminosulfonyl, difluoromethyl(n-butyl))aminosulfonyl, difluoromethyl(sec-butyl)aminosulfonyl, difluoromethyl(t-butyl)aminosulfonyl, difluoromethyl(trifluoromethyl)aminosulfonyl, hydroxymethyl(methyl)aminosulfonyl, ethyl(hydroxymethyl)aminosulfonyl, hydroxymethyl(n-propyl)aminosulfonyl, hydroxymethyl(i-propyl)aminosulfonyl, n-butyl(hydroxymethyl)aminosulfonyl, sec-butyl(hydroxymethyl)aminosulfonyl, t-butyl(hydroxymethyl)aminosulfonyl, difluoromethyl(hydroxymethyl)aminosulfonyl, hydroxymethyl(trifluoromethyl)aminosulfonyl, hydroxyethyl(methyl)aminosulfonyl, ethyl(hydroxyethyl)aminosulfonyl, hydroxyethyl(n-propyl)aminosulfonyl, hydroxyethyl(i-propyl)aminosulfonyl, n-butyl(hydroxyethyl)aminosulfonyl, sec-butyl(hydroxyethyl)aminosulfonyl, t-butyl(hydroxyethyl)aminosulfonyl, difluoromethyl(hydroxyethyl)aminosulfonyl, hydroxyethyl(trifluoromethyl)aminosulfonyl, hydroxypropyl(methyl)aminosulfonyl, ethyl(hydroxypropyl)aminosulfonyl, hydroxypropyl(n-propyl)aminosulfonyl, hydroxypropyl(i-propyl)aminosulfonyl, n-butyl(hydroxypropyl)aminosulfonyl, sec-butyl(hydroxypropyl)aminosulfonyl, t-butyl(hydroxypropyl)aminosulfonyl, difluoromethyl(hydroxypropyl)aminosulfonyl and hydroxypropyl(trifluoromethyl)aminosulfonyl.

As used herein, the term alkylsulfamoyl embraces radicals containing an optionally substituted, linear or branched alkyl radical of 1 to 10 carbon atoms and attached to the nitrogen of a-NSO₂— radical. More preferred alkylsulfamoyl radicals are “lower alkylsulfamoyl” radicals having 1 to 8, preferably 1 to 6 and more preferably 1 to 4 carbon atoms.

An alkylsulfamoyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkylsulfamoyl group are themselves unsubstituted.

Preferred optionally substituted alkylsulfamoyl radicals include methylsulfamoyl, ethylsulfamoyl, n-propylsulfamoyl, i-propylsulfamoyl, n-butylsulfamoyl, sec-butylsulfamoyl, t-butylsulfamoyl, trifluoromethylsulfamoyl, difluoromethylsulfamoyl, hydroxymethylsulfamoyl, 2-hydroxyethylsulfamoyl and 2-hydroxypropylsulfamoyl.

As used herein, the term alkylsulfamido embraces radicals containing an optionally substituted, linear or branched alkyl radicals of 1 to 10 carbon atoms and attached to one of the nitrogen atoms of a —NHSO₂NH— radical. More preferred alkylsulfamido radicals are “lower alkylsulfamido” radicals having 1 to 8, preferably 1 to 6 and more preferably 1 to 4 carbon atoms.

An alkylsulfamido group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkylsulfamido group are themselves unsubstituted.

Preferred optionally substituted alkylsulfamido radicals include methylsulfamido, ethylsulfamido, n-propylsulfamido, i-propylsulfamido, n-butylsulfamido, sec-butylsulfamido, t-butylsulfamido, trifluoromethylsulfamido, difluoromethylsulfamido, hydroxymethylsulfamido, 2-hydroxyethylsulfamido and 2-hydroxysulfamido.

As used herein, the term N′-alkylureido embraces radicals containing an optionally substituted, linear or branched alkyl radical of 1 to 10 carbon atoms attached to the terminal nitrogen of a —NHCONH— radical. More preferred N′-alkylureido radicals are “lower N′-alkylureido” radicals in which the alkyl moiety has 1 to 8, preferably 1 to 6 and more preferably 1 to 4 carbon atoms.

An N′-alkylureido group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an N′-alkylureido group are themselves unsubstituted.

Preferred optionally substituted N′-alkylureido radicals include N′-methylureido, N′-ethylureido, N′-n-propylureido, N′-i-propylureido, N′-n-butylureido, N′-sec-butylureido, N′-t-butylureido, N′-trifluoromethylureido, N′-difluoromethylureido, N′-hydroxymethylureido, N′-2-hydroxyethylureido and N′-2-hydroxypropylureido.

As used herein, the term N′,N′-dialkylureido embraces radicals containing a radical —NHCON where the terminal nitrogen is attached to two optionally substituted, linear or branched alkyl radicals of 1 to 10 carbon atoms. More preferred N′,N′-dialkylureido radicals are “lower N′,N′-dialkylureido” radicals having 1 to 8, preferably 1 to 6 and more preferably 1 to 4 carbon atoms in each alkyl radical.

A N′,N′-dialkylureido group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an N′,N′-dialkylureido group are themselves unsubstituted.

Preferred optionally substituted N′,N′-dialkylureido radicals include N′,N′-dimethylureido, N′,N′-diethylureido, N′-methyl, N′-ethylureido, N′,N′-di(n-propyl)ureido, N′-n-propyl, N′-methylureido, N′-n-propyl, N′-ethylureido, N′,N′-di(i-propyl)ureido, N′-i-propyl, N′-methylureido, N′-i-propyl, N′-ethylureido, N′,N′-di(n-butyl)ureido, N′-n-butyl, N′-methylureido, N′-n-butyl, N′-ethylureido, N′-n-butyl, N′-(i-propyl)ureido, N′,N′-di(sec-butyl)ureido, N′-sec-butyl, N′-methylureido, N′-sec-butyl, N′-ethylureido, N′-sec-butyl, N′-(n-propyl)ureido, N′-sec-butyl, N′(i-propyl)ureido, N′,N′-di(t-butyl)ureido, N′-t-butyl, N′-methylureido, N′-t-butyl, N′-ethylureido, N′-t-butyl, N′-(n-propyl)ureido, N′-t-butyl, N′-(i-propyl)ureido, N′-trifluoromethyl, N′-methylureido, N′-trifluoromethyl, N′-ethylureido, N′-trifluoromethyl, N′-(n-propyl)ureido, N′-trifluoromethyl, N′-(i-propyl)ureido, N′-trifluoromethyl, N′-(n-butyl)ureido, N′-trifluoromethyl, N′-(sec-butyl)ureido, N′-difluoromethyl, N′-methylureido, N′-difluoromethyl, N′-ethylureido, N′-difluoromethyl, N′(n-propyl)ureido, N′-difluoromethyl, N′-(i-propyl)ureido, N′-difluoromethyl, N′-(n-butyl)ureido, N′-difluoromethyl, N′-(sec-butyl)ureido, N′-difluoromethyl, N′-(t-butyl)ureido, N′-difluoromethyl, N′-trifluoromethylureido, N′-hydroxymethyl, N′-methylureido, N′-ethyl, N′-hydroxymethylureido, N′-hydroxymethyl, N′-(n-propyl)ureido, N′-hydroxymethyl, N′-(i-propyl)ureido, N′-n-butyl, N′-hydroxymethylureido, N′-sec-butyl, N′-hydroxymethylureido, N′-t-butyl, N′-hydroxymethylureido, N′-difluoromethyl, N′-hydroxymethylureido, N′hydroxymethyl, N′-trifluoromethylureido, N′-hydroxyethyl, N′-methylureido, N′-ethyl, N′-hydroxyethylureido, N′-hydroxyethyl, N′-(n-propyl)ureido, N′-hydroxyethyl, N′-(i-propyl)ureido, N′-(n-butyl), N′-hydroxyethylureido, N′(sec-butyl), N′-hydroxyethylureido, N′-(t-butyl), N′-hydroxyethylureido, N′-difluoromethyl, N′-hydroxyethylureido, N′-hydroxyethyl, N′-trifluoromethylureido, N′-hydroxypropyl, N′-methylureido, N′-ethyl, N′-hydroxypropylureido, N′-hydroxypropyl, N′-(n-propyl)ureido, N′-hydroxypropyl, N′-(i-propyl)ureido, N′-(n-butyl), N′-hydroxypropylureido, N′(sec-butyl), N′-hydroxypropylureido, N′(t-butyl), N′-hydroxypropylureido, N′-difluoromethyl, N′-hydroxypropylureido y N′-hydroxypropyl, N′-trifluoromethylureido.

As used herein, the term acyl embraces optionally substituted, linear or branched radicals having 2 to 20 carbon atoms or, preferably 2 to 12 carbon atoms attached to a carbonyl radical. More preferably acyl radicals are “lower acyl” radicals of formula —COR, wherein R is a hydrocarbon group, preferably an alkyl group, having 2 to 8, preferably 2 to 6 and more preferably 2 to 4 carbon atoms.

An acyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an acyl group are themselves unsubstituted.

Preferred optionally substituted acyl radicals include acetyl, propionyl, butiryl, isobutiryl, isovaleryl, pivaloyil, valeryl, lauryl, myristyl, stearyl and palmityl,

As used herein, the term aryl radical embraces typically a C₅-C₁₄ monocyclic or polycyclic aryl radical such as phenyl, naphthyl, anthranyl and phenanthryl. Phenyl is preferred.

A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁-C₄ alkyl groups, C₁-C₄ alkoxy groups and C₁-C₄ hydroxyalkyl groups. When an aryl radical carries 2 or more substituents, the substituents may be the same or different. Unless otherwise specified, the substituents on an aryl group are typically themselves unsubstituted.

As used herein, the term heteroaryl radical embraces typically a 5- to 14-membered ring system, preferably a 5- to 10-membered ring system, comprising at least one heteroaromatic ring and containing at least one heteroatom selected from O, S and N. A heteroaryl radical may be a single ring or two or more fused rings wherein at least one ring contains a heteroatom.

A said optionally substituted heteroaryl radical is typically unsubstituted or substituted with 1, 2 or 3 substituents which may be the same or different. The substituents are preferably selected from halogen atoms, preferably fluorine, chlorine or bromine atoms, alkoxycarbonyl groups in which the alkyl moiety has from 1 to 4 carbon atoms, nitro groups, hydroxy groups, C₁-C₄ alkyl groups and C₁-C₄ alkoxy groups. When an heteroaryl radical carries 2 or more substituents, the substituents may be the same or different. Unless otherwise specified, the substituents on a heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, thienopyridinyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, thienopyridinyl, pyrazolyl, 2H-pyrazolo[3,4-d]pyrimidinyl, 1H-pyrazolo[3,4-d]pyrimidinyl, thieno[2,3-d]pyrimidnyl and the various pyrrolopyridyl radicals.

Oxadiazolyl, oxazolyl, pyridyl, pyrrolyl, imidazolyl, thiazolyl, thiadiazolyl, thienyl, furanyl, quinolinyl, isoquinolinyl, thienopyridinyl, indolyl, benzoxazolyl, naphthyridinyl, benzofuranyl, pyrazinyl, pyrimidinyl and the various pyrrolopyridyl radicals are preferred.

As used herein, the term cycloalkyl embraces saturated carbocyclic radicals and, unless otherwise specified, a cycloalkyl radical typically has from 3 to 7 carbon atoms.

A cycloalkyl radical is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. When a cycloalkyl radical carries 2 or more substituents, the substituents may be the same or different. Typically the substituents on a cycloalkyl group are themselves unsubstituted.

Examples include cyclopropyl, cyclobutyl, cyclopentyl, cyclohexyl and cycloheptyl. It is preferably cyclopropyl, cyclopentyl and cyclohexyl.

As used herein, the term cycloalkenyl embraces partially unsaturated carbocyclic radicals and, unless otherwise specified, a cycloalkenyl radical typically has from 3 to 7 carbon atoms.

A cycloalkenyl radical is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. When a cycloalkenyl radical carries 2 or more substituents, the substituents may be the same or different. Typically, the substituents on a cycloalkenyl group are themselves unsubstituted.

Examples include cyclobutenyl, cyclopentenyl, cyclohexenyl and cycloheptenyl. Cyclopentenyl and cyclohexenyl are preferred.

As used herein, the term heterocyclyl radical embraces typically a non-aromatic, saturated or unsaturated C₃-C₁₀ carbocyclic ring system, such as a 5, 6 or 7 membered radical, in which one or more, for example 1, 2, 3 or 4 of the carbon atoms preferably 1 or 2 of the carbon atoms are replaced by a heteroatom selected from N, O and S. Saturated heterocyclyl radicals are preferred. A heterocyclic radical may be a single ring or two or more fused rings wherein at least one ring contains a heteroatom. When a heterocyclyl radical carries 2 or more substituents, the substituents may be the same or different.

A said optionally substituted heterocyclyl radical is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a heterocyclyl radical are themselves unsubstituted.

Examples of heterocyclic radicals include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, cromanyl, isocromanyl, imidazolidinyl, imidazolyl, oxiranyl, azaridinyl, 4,5-dihydro-oxazolyl, 2-benzofuran-1(3H)-one, 1,3-dioxol-2-one and 3-aza-tetrahydrofuranyl.

Where a heterocyclyl radical carries 2 or more substituents, the substituents may be the same or different.

As used herein, some of the atoms, radicals, moieties, chains and cycles present in the general structures of the invention are “optionally substituted”. This means that these atoms, radicals, moieties, chains and cycles can be either unsubstituted or substituted in any position by one or more, for example 1, 2, 3 or 4, substituents, whereby the hydrogen atoms bound to the unsubstituted atoms, radicals, moieties, chains and cycles are replaced by chemically acceptable atoms, radicals, moieties, chains and cycles. When two or more substituents are present, each substituent may be the same or different. The substituents are typically themselves unsubstituted.

Typically when a cyclic radical is bridged by an alkylene or alkylenedioxy radical, the bridging alkylene radical is attached to the ring at non-adjacent atoms.

As used herein, the term halogen atom embraces chlorine, fluorine, bromine and iodine atoms. A halogen atom is typically a fluorine, chlorine or bromine atom, most preferably chlorine or fluorine. The term halo when used as a prefix has the same meaning.

As used herein, an acylamino group is typically a said acyl group attached to an amino group.

As used herein an alkylenedioxy group is typically —O—R—O—, wherein R is a said alkylene group.

As used herein, an alkoxycarbonyl group is typically a said alkoxy group attached to a said carbonyl group.

As used herein, an acyloxy group is typically a said acyl group attached to an oxygen atom.

As used herein, a cycloalkoxy group is typically a said cycloalkyl group attached to an oxygen atom.

Compounds containing one or more chiral centre may be used in enantiomerically or diastereoisomerically pure form, or in the form of a mixture of isomers.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

As used herein, an N-oxide is formed from the tertiary basic amines or imines present in the molecule, using a convenient oxidising agent.

According to one embodiment of the present invention in the compounds of formula (I) R¹ is selected from the group consisting of hydrogen atoms and lower alkyl groups, which are optionally substituted by one or more substituents selected from halogen atoms and hydroxy, alkoxy, alkylthio, hydroxycarbonyl and alkoxycarbonyl groups.

According to another embodiment of the present invention in the compounds of formula (I) R² is an heteroaryl group which is optionally substituted by one or more substituents selected from halogen atoms and hydroxy, lower alkyl, hydroxyalkyl, hydroxycarbonyl, alkoxy, alkylenedioxy, alkoxycarbonyl, aryloxy, acyl, acyloxy, alkylthio, arylthio, amino, nitro, cyano, mono- or di-alkylamino, acylamino, carbamoyl or mono- or di-alkylcarbamoyl, difluoromethyl, trifluoromethyl, difluoromethoxy or trifluoromethoxy groups. It is preferred that R² is an heteroaryl group which is optionally substituted by one or more substituents selected from halogen atoms and hydroxy, hydroxyalkyl, hydroxycarbonyl, alkoxy, alkylenedioxy, alkoxycarbonyl, aryloxy, acyl, acyloxy, alkylthio, arylthio, amino, nitro, cyano, mono- or di-alkylamino, acylamino, carbamoyl or mono- or di-alkylcarbamoyl, difluoromethyl, trifluoromethyl, difluoromethoxy or trifluoromethoxy groups. It is further preferred that R² is a N-containing heteroaryl group and still more preferred that R² is optionally substituted by one or more substituents selected from halogen atoms and lower alkyl groups.

According to still another embodiment of the present invention in the compounds of formula (I) R³ represents:

G-L1-(CRR′)ₙ—

wherein
n is an integer from 0 to 3, preferably from 1 to 3
R and R′ are independently selected from the group consisting of hydrogen atoms and lower alkyl groups
L1 is a linker selected from the group consisting of a direct bond, —CO—, —O(CO)—, —O(CO)O— and —(CO)O—; and
G is selected from hydrogen atoms and alkyl, cycloalkyl, heterocyclyl, aryl and heteroaryl groups said groups being optionally substituted with one or more substituents selected from:
halogen atoms;
alkyl and alkenyl groups, which are optionally substituted by one or more substituents selected from halogen atoms; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono- or di-alkylaminosulfonyl, cyano, difluoromethoxy or trifluoromethoxy groups;

It is particularly advantageous that when n is zero, L1 is a direct bond and G is different from a hydrogen atom.

According to still another embodiment of the present invention in the compounds of formula (I) R³ represents:

G-L1-(CRR′)ₙ—

wherein
n is an integer from 0 to 3, preferably from 1 to 3
R and R′ are independently selected from the group consisting of hydrogen atoms and methyl groups
L1 is a linker selected from the group consisting of a direct bond, —CO—, —O(CO)—, O(CO)O— and —(CO)O—; and
G is selected from alkyl, cycloalkyl, heterocyclyl, aryl and heteroaryl groups said groups being optionally substituted with one or more halogen atoms;

According to still another embodiment of the present invention in the compounds of formula (I) R³ represents:

G-L1-(CRR′)ₙ—

wherein
n is 0 or 1, preferably 1
R is a hydrogen atom
R′ is a hydrogen atom or a methyl group
L1 is a linker selected from the group consisting of a direct bond, —O(CO)O— and —(CO)O—; and
G is selected from alkyl and cycloalkyl groups said groups being optionally substituted with one halogen atoms.

According to another embodiment of the present invention in the compounds of formula (I) R⁴ represents a phenyl, pyridyl or thienyl group, which is optionally substituted by one or more substituents selected from:
halogen atoms;
alkyl groups, which are optionally substituted by one or more substituents selected from halogen atoms and hydroxy, hydroxyalkyl, alkoxy, alkylthio, mono- or di-alkylamino, acylamino, hydroxycarbonyl, alkoxycarbonyl, carbamoyl, mono- or di-alkylcarbamoyl groups; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono- or di-alkylaminosulfonyl, cyano, difluoromethoxy or trifluoromethoxy groups;

It is preferred that R⁴ is optionally substituted by one or more substituents selected from halogen atoms and lower alkyl groups. Most preferably R⁴ is a phenyl group.

In another embodiment of the invention the compounds of formula (I):

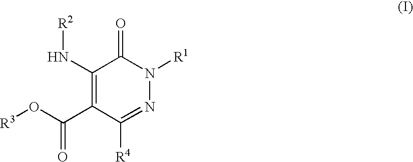

wherein
R¹ represents an ethyl group
R² is a N-containing heteroaryl group optionally substituted by one substituent selected from halogen atoms and lower alkyl groups.
R³ represents:

G-L1-(CRR′)ₙ—

wherein
n is 0 or 1, preferably 1
R is a hydrogen atom
R′ is a hydrogen atom or a methyl group
L1 is a linker selected from the group consisting of a direct bond, —O(CO)O— and —(CO)O—; and
G is selected from alkyl and cycloalkyl groups said groups being optionally substituted with one halogen atoms; and
   R⁴ represents a phenyl group
   and the pharmaceutically acceptable salts or N-oxides are preferred for use in a formulation for topical application.
   Particular individual compounds of the invention include:
4-(methoxycarbonyl)benzyl 1-ethyl-6-oxo-3-phenyl-5-(quinolin-5-ylamino)-1,6-dihydropyridazine-4-carboxylate
benzyl 1-ethyl-6-oxo-3-phenyl-5-(quinolin-5-ylamino)-1,6-dihydropyridazine-4-carboxylate
2-(benzyloxy)-2-oxoethyl 1-ethyl-6-oxo-3-phenyl-5-(quinolin-5-ylamino)-1,6-dihydropyridazine-4-carboxylate
2-ethoxy-2-oxoethyl 1-ethyl-6-oxo-3-phenyl-5-(quinolin-5-ylamino)-1,6-dihydropyridazine-4-carboxylate
2-oxo-2-pyrrolidin-1-ylethyl 1-ethyl-6-oxo-3-phenyl-5-(quinolin-5-ylamino)-1,6-dihydropyridazine-4-carboxylate
3-amino-3-oxopropyl 1-ethyl-6-oxo-3-phenyl-5-(quinolin-5-ylamino)-1,6-dihydropyridazine-4-carboxylate
2-(dimethylamino)ethyl 1-ethyl-6-oxo-3-phenyl-5-(quinolin-5-ylamino)-1,6-dihydropyridazine-4-carboxylate
2-[(tert-butoxycarbonyl)amino]ethyl 1-ethyl-6-oxo-3-phenyl-5-(quinolin-5-ylamino)-1,6-dihydropyridazine-4-carboxylate
2-(acetyloxy)ethyl 1-ethyl-6-oxo-3-phenyl-5-(quinolin-5-ylamino)-1,6-dihydropyridazine-4-carboxylate
3-fluorobenzyl 1-ethyl-6-oxo-3-phenyl-5-(quinolin-5-ylamino)-1,6-dihydropyridazine-4-carboxylate
[(2,2-dimethylpropanoyl)oxy]methyl 1-ethyl-6-oxo-3-phenyl-5-(quinolin-5-ylamino)-1,6-dihydropyridazine-4-carboxylate
2-oxo-2-pyridin-4-ylethyl 1-ethyl-6-oxo-3-phenyl-5-(quinolin-5-ylamino)-1,6-dihydropyridazine-4-carboxylate
2-(dimethylamino)-2-oxoethyl 1-ethyl-6-oxo-3-phenyl-5-(quinolin-5-ylamino)-1,6-dihydropyridazine-4-carboxylate
2-aminoethyl 1-ethyl-6-oxo-3-phenyl-5-(quinolin-5-ylamino)-1,6-dihydropyridazine-4-carboxylate
ethyl 1-ethyl-6-oxo-3-phenyl-5-(pyridin-3-ylamino)-1,6-dihydropyridazine-4-carboxylate
2-(benzyloxy)-2-oxoethyl 1-ethyl-6-oxo-3-phenyl-5-(pyridin-3-ylamino)-1,6-dihydropyridazine-4-carboxylate
(butyryloxy)methyl 1-ethyl-6-oxo-3-phenyl-5-(pyridin-3-ylamino)-1,6-dihydropyridazine-4-carboxylate
3-oxo-1,3-dihydro-2-benzofuran-1-yl 1-ethyl-6-oxo-3-phenyl-5-(pyridin-3-ylamino)-1,6-dihydropyridazine-4-carboxylate
(acetyloxy)methyl 1-ethyl-6-oxo-3-phenyl-5-(pyridin-3-ylamino)-1,6-dihydropyridazine-4-carboxylate
1-(acetyloxy)ethyl 1-ethyl-6-oxo-3-phenyl-5-(pyridin-3-ylamino)-1,6-dihydropyridazine-4-carboxylate
2-(dimethylamino)-2-oxoethyl 1-ethyl-6-oxo-3-phenyl-5-(pyridin-3-ylamino)-1,6-dihydropyridazine-4-carboxylate
benzyl 1-ethyl-6-oxo-3-phenyl-5-(pyridin-3-ylamino)-1,6-dihydropyridazine-4-carboxylate
[(2,2-dimethylpropanoyl)oxy]methyl 1-ethyl-6-oxo-3-phenyl-5-(pyridin-3-ylamino)-1,6-dihydropyridazine-4-carboxylate
1-(acetyloxy)-1-methylethyl 1-ethyl-6-oxo-3-phenyl-5-(pyridin-3-ylamino)-1,6-dihydropyridazine-4-carboxylate
ethyl 1-ethyl-5-[(4-methylpyridin-3-yl)amino]-6-oxo-3-phenyl-1,6-dihydropyridazine-4-carboxylate
[(2,2-dimethylpropanoyl)oxy]methyl 1-ethyl-5-[(4-methylpyridin-3-yl)amino]-6-oxo-3-phenyl-1,6-dihydropyridazine-4-carboxylate
1-[(ethoxycarbonyl)oxy]ethyl 1-ethyl-5-[(4-methylpyridin-3-yl)amino]-6-oxo-3-phenyl-1,6-dihydropyridazine-4-carboxylate
2-(benzyloxy)-2-oxoethyl 1-ethyl-5-[(4-methylpyridin-3-yl)amino]-6-oxo-3-phenyl-1,6-dihydropyridazine-4-carboxylate
1-[(ethoxycarbonyl)oxy]ethyl 1-ethyl-6-oxo-3-phenyl-5-(pyridin-3-ylamino)-1,6-dihydropyridazine-4-carboxylate
ethyl 1-ethyl-5-(isoquinolin-4-ylamino)-6-oxo-3-phenyl-1,6-dihydropyridazine-4-carboxylate
[(2,2-dimethylpropanoyl)oxy]methyl 1-ethyl-5-(isoquinolin-4-ylamino)-6-oxo-3-phenyl-1,6-dihydropyridazine-4-carboxylate
1-(acetyloxy)ethyl 1-ethyl-5-(isoquinolin-4-ylamino)-6-oxo-3-phenyl-1,6-dihydropyridazine-4-carboxylate
({[1-ethyl-6-oxo-3-phenyl-5-(pyridin-3-ylamino)-1,6-dihydropyridazin-4-yl]carbonyl}oxy)acetic acid
ethyl 1-ethyl-3-(3-methylphenyl)-6-oxo-5-(pyridin-3-ylamino)-1,6-dihydropyridazine-4-carboxylate
[(2,2-dimethylpropanoyl)oxy]methyl 1-ethyl-3-(3-methylphenyl)-6-oxo-5-(pyridin-3-ylamino)-1,6-dihydropyridazine-4-carboxylate
ethyl 1-ethyl-3-(3-fluorophenyl)-6-oxo-5-(pyridin-3-ylamino)-1,6-dihydropyridazine-4-carboxylate
(butyryloxy)methyl 1-ethyl-3-(3-fluorophenyl)-6-oxo-5-(pyridin-3-ylamino)-1,6-dihydropyridazine-4-carboxylate
ethyl 1-ethyl-3-(4-fluorophenyl)-6-oxo-5-(pyridin-3-ylamino)-1,6-dihydropyridazine-4-carboxylate
(butyryloxy)methyl 1-ethyl-3-(4-fluorophenyl)-6-oxo-5-(pyridin-3-ylamino)-1,6-dihydropyridazine-4-carboxylate
ethyl 5-[(2-chloropyridin-3-yl)amino]-1-ethyl-6-oxo-3-phenyl-1,6-dihydropyridazine-4-carboxylate
methyl 1-ethyl-6-oxo-3-phenyl-5-(pyridin-3-ylamino)-1,6-dihydropyridazine-4-carboxylate
methyl 1-ethyl-6-oxo-3-phenyl-5-(quinolin-5-ylamino)-1,6-dihydropyridazine-4-carboxylate
ethyl 1-ethyl-6-oxo-5-(pyridin-3-ylamino)-3-(2-thienyl)-1,6-dihydropyridazine-4-carboxylate
2-(acetyloxy)ethyl 1-ethyl-6-oxo-5-(pyridin-3-ylamino)-3-(2-thienyl)-1,6-dihydropyridazine-4-carboxylate
2-[(tert-butoxycarbonyl)amino]ethyl 1-ethyl-6-oxo-5-(pyridin-3-ylamino)-3-(2-thienyl)-1,6-dihydropyridazine-4-carboxylate
2-ethoxy-2-oxoethyl 1-ethyl-6-oxo-5-(pyridin-3-ylamino)-3-(2-thienyl)-1,6-dihydropyridazine-4-carboxylate
2-(benzyloxy)-2-oxoethyl 1-ethyl-6-oxo-5-(pyridin-3-ylamino)-3-(2-thienyl)-1,6-dihydropyridazine-4-carboxylate
benzyl 1-ethyl-6-oxo-5-(pyridin-3-ylamino)-3-(2-thienyl)-1,6-dihydropyridazine-4-carboxylate
ethyl 1-ethyl-5-(4-methyloyridin-3-ylamino)-6-oxo-3-thien-2-yl-1,6-dihydropyridazin-4-carboxylate
2-(acetyloxy)ethyl 1-ethyl-5-[(4-methylpyridin-3-yl)amino]-6-oxo-3-(2-thienyl)-1,6-dihydropyridazine-4-carboxylate
2-[(tert-butoxycarbonyl)amino]ethyl 1-ethyl-5-[(4-methylpyridin-3-yl)amino]-6-oxo-3-(2-thienyl)-1,6-dihydropyridazine-4-carboxylate
ethyl 1-ethyl-5-(isoquinolin-4-ylamino)-6-oxo-3-(2-thienyl)-1,6-dihydropyridazine-4-carboxylate
2-(acetyloxy)ethyl 1-ethyl-5-(isoquinolin-4-ylamino)-6-oxo-3-(2-thienyl)-1,6-dihydropyridazine-4-carboxylate
2-[(tert-butoxycarbonyl)amino]ethyl 1-ethyl-5-(isoquinolin-4-ylamino)-6-oxo-3-(2-thienyl)-1,6-dihydropyridazine-4-carboxylate
2-ethoxy-2-oxoethyl 1-ethyl-5-(isoquinolin-4-ylamino)-6-oxo-3-(2-thienyl)-1,6-dihydropyridazine-4-carboxylate
2-(benzyloxy)-2-oxoethyl 1-ethyl-5-(isoquinolin-4-ylamino)-6-oxo-3-(2-thienyl)-1,6-dihydropyridazine-4-carboxylate
benzyl 1-ethyl-5-(isoquinolin-4-ylamino)-6-oxo-3-(2-thienyl)-1,6-dihydropyridazine-4-carboxylate
ethyl 1-ethyl-5-(isoquinolin-4-ylamino)-6-oxo-3-(3-thienyl)-1,6-dihydropyridazine-4-carboxylate
2-(acetyloxy)ethyl 1-ethyl-5-(isoquinolin-4-ylamino)-6-oxo-3-(3-thienyl)-1,6-dihydropyridazine-4-carboxylate
2-[(tert-butoxycarbonyl)amino]ethyl 1-ethyl-5-(isoquinolin-4-ylamino)-6-oxo-3-(3-thienyl)-1,6-dihydropyridazine-4-carboxylate
2-ethoxy-2-oxoethyl 1-ethyl-5-(isoquinolin-4-ylamino)-6-oxo-3-(3-thienyl)-1,6-dihydropyridazine-4-carboxylate
2-(benzyloxy)-2-oxoethyl 1-ethyl-5-(isoquinolin-4-ylamino)-6-oxo-3-(3-thienyl)-1,6-dihydropyridazine-4-carboxylate
4-fluorobenzyl 1-ethyl-5-(isoquinolin-4-ylamino)-6-oxo-3-(2-thienyl)-1,6-dihydropyridazine-4-carboxylate
4-(methoxycarbonyl)benzyl 1-ethyl-5-(isoquinolin-4-ylamino)-6-oxo-3-(2-thienyl)-1,6-dihydropyridazine-4-carboxylate
ethyl 1-ethyl-3-(4-methylphenyl)-6-oxo-5-(pyridin-3-ylamino)-1,6-dihydropyridazine-4-carboxylate
ethyl 1-ethyl-5-(isoquinolin-4-ylamino)-3-(4-methylphenyl)-6-oxo-1,6-dihydropyridazine-4-carboxylate
ethyl 1-ethyl-3-(4-methylphenyl)-5-[(4-methylpyridin-3-yl)amino]-6-oxo-1,6-dihydropyridazine-4-carboxylate
[(2,2-dimethylpropanoyl)oxy]methyl 1-ethyl-3-(4-methylphenyl)-6-oxo-5-(pyridin-3-ylamino)-1,6-dihydropyridazine-4-carboxylate
[(2,2-dimethylpropanoyl)oxy]methyl 1-ethyl-5-(isoquinolin-4-ylamino)-3-(4-methylphenyl)-6-oxo-1,6-dihydropyridazine-4-carboxylate
[(2,2-dimethylpropanoyl)oxy]methyl 1-ethyl-3-(4-methylphenyl)-5-[(4-methylpyridin-3-yl)amino]-6-oxo-1,6-dihydropyridazine-4-carboxylate
1-[(isopropoxycarbonyl)oxy]ethyl 1-ethyl-6-oxo-3-phenyl-5-(pyridin-3-ylamino)-1,6-dihydropyridazine-4-carboxylate
1-[(isopropoxycarbonyl)oxy]ethyl 1-ethyl-5-(isoquinolin-4-ylamino)-6-oxo-3-phenyl-1,6-dihydropyridazine-4-carboxylate
1-[(isopropoxycarbonyl)oxy]ethyl 1-ethyl-5-[(4-methylpyridin-3-yl)amino]-6-oxo-3-phenyl-1,6-dihydropyridazine-4-carboxylate
1-{[(cyclohexyloxy)carbonyl]oxy}ethyl 1-ethyl-6-oxo-3-phenyl-5-(pyridin-3-ylamino)-1,6-dihydropyridazine-4-carboxylate
1-{[(cyclohexyloxy)carbonyl]oxy}ethyl 1-ethyl-5-(isoquinolin-4-ylamino)-6-oxo-3-phenyl-1,6-dihydropyridazine-4-carboxylate
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate
ethyl 1-ethyl-6-oxo-3-phenyl-5-(thieno[2,3-c]pyridin-3-ylamino)-1,6-dihydropyridazine-4-carboxylate
ethyl 1-ethyl-6-oxo-3-phenyl-5-(thieno[2,3-b]pyridin-3-ylamino)-1,6-dihydropyridazine-4-carboxylate
[(2,2-dimethylpropanoyl)oxy]methyl 1-ethyl-6-oxo-3-phenyl-5-(thieno[2,3-b]pyridin-3-ylamino)-1,6-dihydropyridazine-4-carboxylate
7-ethoxy-7-oxoheptyl 1-ethyl-5-(isoquinolin-4-ylamino)-6-oxo-3-(3-thienyl)-1,6-dihydropyridazine-4-carboxylate
6-ethoxy-6-oxohexyl 1-ethyl-5-(isoquinolin-4-ylamino)-6-oxo-3-(3-thienyl)-1,6-dihydropyridazine-4-carboxylate
3-amino-3-oxopropyl 1-ethyl-5-(isoquinolin-4-ylamino)-6-oxo-3-(3-thienyl)-1,6-dihydropyridazine-4-carboxylate
2-ethoxy-2-oxoethyl 1-ethyl-5-[(4-methylpyridin-3-yl)amino]-6-oxo-3-(2-thienyl)-1,6-dihydropyridazine-4-carboxylate
2-(benzyloxy)-2-oxoethyl 1-ethyl-5-[(4-methylpyridin-3-yl)amino]-6-oxo-3-(2-thienyl)-1,6-dihydropyridazine-4-carboxylate
1-{[(1-ethylpropoxy)carbonyl]oxy}ethyl 1-ethyl-6-oxo-3-phenyl-5-(pyridin-3-ylamino)-1,6-dihydropyridazine-4-carboxylate
1-{[(1-ethylpropoxy)carbonyl]oxy}ethyl 1-ethyl-5-[(4-methylpyridin-3-yl)amino]-6-oxo-3-phenyl-1,6-dihydropyridazine-4-carboxylate
1-{[(1-ethylpropoxy)carbonyl]oxy}ethyl 1-ethyl-5-(isoquinolin-4-ylamino)-6-oxo-3-phenyl-1,6-dihydropyridazine-4-carboxylate
(butyryloxy)methyl 1-ethyl-5-(isoquinolin-4-ylamino)-6-oxo-3-phenyl-1,6-dihydropyridazine-4-carboxylate
(acetyloxy)methyl 1-ethyl-5-[(4-methylpyridin-3-yl)amino]-6-oxo-3-phenyl-1,6-dihydropyridazine-4-carboxylate
benzyl 1-ethyl-5-[(4-methylpyridin-3-yl)amino]-6-oxo-3-(2-thienyl)-1,6-dihydropyridazine-4-carboxylate
4-(methoxycarbonyl)benzyl 1-ethyl-5-[(4-methylpyridin-3-yl)amino]-6-oxo-3-(2-thienyl)-1,6-dihydropyridazine-4-carboxylate
(isobutyryloxy)methyl 1-ethyl-5-(isoquinolin-4-ylamino)-6-oxo-3-phenyl-1,6-dihydropyridazine-4-carboxylate
(isobutyryloxy)methyl 1-ethyl-6-oxo-3-phenyl-5-(pyridin-3-ylamino)-1,6-dihydropyridazine-4-carboxylate
4-fluorobenzyl 1-ethyl-6-oxo-5-(pyridin-3-ylamino)-3-(2-thienyl)-1,6-dihydropyridazine-4-carboxylate
4-(methoxycarbonyl)benzyl 1-ethyl-6-oxo-5-(pyridin-3-ylamino)-3-(2-thienyl)-1,6-dihydropyridazine-4-carboxylate
[(isopropoxycarbonyl)oxy]methyl 1-ethyl-6-oxo-3-phenyl-5-(pyridin-3-ylamino)-1,6-dihydropyridazine-4-carboxylate
7-ethoxy-7-oxoheptyl 1-ethyl-6-oxo-5-(pyridin-3-ylamino)-3-(2-thienyl)-1,6-dihydropyridazine-4-carboxylate
6-ethoxy-6-oxohexyl 1-ethyl-6-oxo-5-(pyridin-3-ylamino)-3-(2-thienyl)-1,6-dihydropyridazine-4-carboxylate
4-fluorobenzyl 1-ethyl-5-[(4-methylpyridin-3-yl)amino]-6-oxo-3-(2-thienyl)-1,6-dihydropyridazine-4-carboxylate
(5-methyl-2-oxo-1,3-dioxol-4-yl)methyl 1-ethyl-6-oxo-3-phenyl-5-(pyridin-3-ylamino)-1,6-dihydropyridazine-4-carboxylate
ethyl 1-ethyl-5-[(4-methylpyridin-3-yl)amino]-6-oxo-3-(3-thienyl)-1,6-dihydropyridazine-4-carboxylate
chloromethyl 1-ethyl-6-oxo-3-phenyl-5-(pyridin-3-ylamino)-1,6-dihydropyridazine-4-carboxylate
{[(cyclohexyloxy)carbonyl]oxy}methyl 1-ethyl-5-[(4-methylpyridin-3-yl)amino]-6-oxo-3-phenyl-1,6-dihydropyridazine-4-carboxylate
(5-methyl-2-oxo-1,3-dioxol-4-yl)methyl 1-ethyl-5-[(4-methylpyridin-3-yl)amino]-6-oxo-3-phenyl-1,6-dihydropyridazine-4-carboxylate
[(2,2-dimethylbutanoyl)oxy]methyl 1-ethyl-6-oxo-3-phenyl-5-(pyridin-3-ylamino)-1,6-dihydropyridazine-4-carboxylate
({N-[(benzyloxy)carbonyl]-L-valyl}oxy)methyl 1-ethyl-6-oxo-3-phenyl-5-(pyridin-3-ylamino)-1,6-dihydropyridazine-4-carboxylate
(5-methyl-2-oxo-1,3-dioxol-4-yl)methyl 1-ethyl-5-(isoquinolin-4-ylamino)-6-oxo-3-phenyl-1,6-dihydropyridazine-4-carboxylate
ethyl 1-ethyl-6-oxo-5-(pyridin-3-ylamino)-3-(3-thienyl)-1,6-dihydropyridazine-4-carboxylate
{[(cyclohexyloxy)carbonyl]oxy}methyl 1-ethyl-5-(isoquinolin-4-ylamino)-6-oxo-3-phenyl-1,6-dihydropyridazine-4-carboxylate
{[(cyclohexyloxy)carbonyl]oxy}methyl 1-ethyl-6-oxo-3-phenyl-5-(pyridin-3-ylamino)-1,6-dihydropyridazine-4-carboxylate
2-(acetyloxy)ethyl 1-ethyl-5-[(4-methylpyridin-3-yl)amino]-6-oxo-3-(3-thienyl)-1,6-dihydropyridazine-4-carboxylate
2-[(tert-butoxycarbonyl)amino]ethyl 1-ethyl-5-[(4-methylpyridin-3-yl)amino]-6-oxo-3-(3-thienyl)-1,6-dihydropyridazine-4-carboxylate
{[(1-ethylpropoxy)carbonyl]oxy}methyl 1-ethyl-6-oxo-3-phenyl-5-(pyridin-3-ylamino)-1,6-dihydropyridazine-4-carboxylate
(isobutyryloxy)methyl 1-ethyl-5-[(4-methylpyridin-3-yl)amino]-6-oxo-3-phenyl-1,6-dihydropyridazine-4-carboxylate
2-(acetyloxy)ethyl 1-ethyl-6-oxo-5-(pyridin-3-ylamino)-3-(3-thienyl)-1,6-dihydropyridazine-4-carboxylate
2-[(tert-butoxycarbonyl)amino]ethyl 1-ethyl-6-oxo-5-(pyridin-3-ylamino)-3-(3-thienyl)-1,6-dihydropyridazine-4-carboxylate
2-ethoxy-2-oxoethyl 1-ethyl-6-oxo-5-(pyridin-3-ylamino)-3-(3-thienyl)-1,6-dihydropyridazine-4-carboxylate
[(isopropoxycarbonyl)oxy]methyl 1-ethyl-5-(isoquinolin-4-ylamino)-6-oxo-3-phenyl-1,6-dihydropyridazine-4-carboxylate
2-(benzyloxy)-2-oxoethyl 1-ethyl-6-oxo-5-(pyridin-3-ylamino)-3-(3-thienyl)-1,6-dihydropyridazine-4-carboxylate
2-ethoxy-2-oxoethyl 1-ethyl-5-[(4-methylpyridin-3-yl)amino]-6-oxo-3-(3-thienyl)-1,6-dihydropyridazine-4-carboxylate
2-(benzyloxy)-2-oxoethyl 1-ethyl-5-[(4-methylpyridin-3-yl)amino]-6-oxo-3-(3-thienyl)-1,6-dihydropyridazine-4-carboxylate
{[(1-ethylpropoxy)carbonyl]oxy}methyl 1-ethyl-5-(isoquinolin-4-ylamino)-6-oxo-3-phenyl-1,6-dihydropyridazine-4-carboxylate
benzyl 1-ethyl-5-[(4-methylpyridin-3-yl)amino]-6-oxo-3-(3-thienyl)-1,6-dihydropyridazine-4-carboxylate
benzyl 1-ethyl-6-oxo-5-(pyridin-3-ylamino)-3-(3-thienyl)-1,6-dihydropyridazine-4-carboxylate
[(isopropoxycarbonyl)oxy]methyl 1-ethyl-5-[(4-methylpyridin-3-yl)amino]-6-oxo-3-phenyl-1,6-dihydropyridazine-4-carboxylate
6-ethoxy-6-oxohexyl 1-ethyl-5-(isoquinolin-4-ylamino)-6-oxo-3-(2-thienyl)-1,6-dihydropyridazine-4-carboxylate
7-ethoxy-7-oxoheptyl 1-ethyl-5-(isoquinolin-4-ylamino)-6-oxo-3-(2-thienyl)-1,6-dihydropyridazine-4-carboxylate
({[1-ethyl-6-oxo-3-phenyl-5-(pyridin-3-ylamino)-1,6-dihydropyridazin-4-yl]carbonyl}oxy)methyl N-(tert-butoxycarbonyl)-L-leucinate
2-methoxy-2-oxoethyl 1-ethyl-5-(isoquinolin-4-ylamino)-6-oxo-3-phenyl-1,6-dihydropyridazine-4-carboxylate
4-fluorobenzyl 1-ethyl-6-oxo-5-(pyridin-3-ylamino)-3-(3-thienyl)-1,6-dihydropyridazine-4-carboxylate
4-(methoxycarbonyl)benzyl 1-ethyl-6-oxo-5-(pyridin-3-ylamino)-3-(3-thienyl)-1,6-dihydropyridazine-4-carboxylate
(butyryloxy)methyl 1-ethyl-5-[(4-methylpyridin-3-yl)amino]-6-oxo-3-phenyl-1,6-dihydropyridazine-4-carboxylate
6-ethoxy-6-oxohexyl 1-ethyl-5-[(4-methylpyridin-3-yl)amino]-6-oxo-3-(2-thienyl)-1,6-dihydropyridazine-4-carboxylate
{[(1-ethylpropoxy)carbonyl]oxy}methyl 1-ethyl-5-[(4-methylpyridin-3-yl)amino]-6-oxo-3-phenyl-1,6-dihydropyridazine-4-carboxylate
7-ethoxy-7-oxoheptyl 1-ethyl-5-[(4-methylpyridin-3-yl)amino]-6-oxo-3-(2-thienyl)-1,6-dihydropyridazine-4-carboxylate
({[1-ethyl-6-oxo-3-phenyl-5-(pyridin-3-ylamino)-1,6-dihydropyridazin-4-yl]carbonyl}oxy)methyl L-leucinate
benzyl 1-ethyl-5-(isoquinolin-4-ylamino)-6-oxo-3-(3-thienyl)-1,6-dihydropyridazine-4-carboxylate
3-amino-3-oxopropyl 1-ethyl-5-[(4-methylpyridin-3-yl)amino]-6-oxo-3-(3-thienyl)-1,6-dihydropyridazine-4-carboxylate
4-fluorobenzyl 1-ethyl-5-[(4-methylpyridin-3-yl)amino]-6-oxo-3-(3-thienyl)-1,6-dihydropyridazine-4-carboxylate
4-(methoxycarbonyl)benzyl 1-ethyl-5-[(4-methylpyridin-3-yl)amino]-6-oxo-3-(3-thienyl)-1,6-dihydropyridazine-4-carboxylate
[(2-methylbutanoyl)oxy]methyl 1-ethyl-6-oxo-3-phenyl-5-(pyridin-3-ylamino)-1,6-dihydropyridazine-4-carboxylate
4-(methoxycarbonyl)benzyl 1-ethyl-5-(isoquinolin-4-ylamino)-6-oxo-3-(3-thienyl)-1,6-dihydropyridazine-4-carboxylate
7-ethoxy-7-oxoheptyl 1-ethyl-5-[(4-methylpyridin-3-yl)amino]-6-oxo-3-(3-thienyl)-1,6-dihydropyridazine-4-carboxylate
7-ethoxy-7-oxoheptyl 1-ethyl-6-oxo-5-(pyridin-3-ylamino)-3-(3-thienyl)-1,6-dihydropyridazine-4-carboxylate
4-fluorobenzyl 1-ethyl-5-(isoquinolin-4-ylamino)-6-oxo-3-(3-thienyl)-1,6-dihydropyridazine-4-carboxylate
6-ethoxy-6-oxohexyl 1-ethyl-5-[(4-methylpyridin-3-yl)amino]-6-oxo-3-(3-thienyl)-1,6-dihydropyridazine-4-carboxylate
6-ethoxy-6-oxohexyl 1-ethyl-6-oxo-5-(pyridin-3-ylamino)-3-(3-thienyl)-1,6-dihydropyridazine-4-carboxylate
1-{[(cyclohexyloxy)carbonyl]oxy}ethyl 1-ethyl-5-(1,7-naphthyridin-5-ylamino)-6-oxo-3-phenyl-1,6-dihydropyridazine-4-carboxylate
benzyl 1-ethyl-6-oxo-3-pyridin-4-yl-5-(pyridin-3-ylamino)-1,6-dihydropyridazine-4-carboxylate
({[1-ethyl-6-oxo-3-phenyl-5-(pyridin-3-ylamino)-1,6-dihydropyridazin-4-yl]carbonyl}oxy)methyl morpholine-4-carboxylate
{[(methylamino)carbonyl]oxy}methyl 1-ethyl-6-oxo-3-phenyl-5-(pyridin-3-ylamino)-1,6-dihydropyridazine-4-carboxylate
{[(dimethylamino)carbonyl]oxy}methyl 1-ethyl-6-oxo-3-phenyl-5-(pyridin-3-ylamino)-1,6-dihydropyridazine-4-carboxylate
(acetyloxy)methyl 1-ethyl-5-(isoquinolin-4-ylamino)-6-oxo-3-phenyl-1,6-dihydropyridazine-4-carboxylate
[(dibutoxyphosphoryl)oxy]methyl 1-ethyl-6-oxo-3-phenyl-5-(pyridin-3-ylamino)-1,6-dihydropyridazine-4-carboxylate
(acetyloxy)methyl 1-ethyl-6-oxo-5-(pyridin-3-ylamino)-3-(2-thienyl)-1,6-dihydropyridazine-4-carboxylate
[(isopropoxycarbonyl)oxy]methyl 1-ethyl-6-oxo-5-(pyridin-3-ylamino)-3-(2-thienyl)-1,6-dihydropyridazine-4-carboxylate
{[(cyclohexyloxy)carbonyl]oxy}methyl 1-ethyl-6-oxo-5-(pyridin-3-ylamino)-3-(2-thienyl)-1,6-dihydropyridazine-4-carboxylate
(acetyloxy)methyl 1-ethyl-5-[(4-methylpyridin-3-yl)amino]-6-oxo-3-(2-thienyl)-1,6-dihydropyridazine-4-carboxylate
[(isopropoxycarbonyl)oxy]methyl 1-ethyl-5-[(4-methylpyridin-3-yl)amino]-6-oxo-3-(2-thienyl)-1,6-dihydropyridazine-4-carboxylate
{[(cyclohexyloxy)carbonyl]oxy}methyl 1-ethyl-5-[(4-methylpyridin-3-yl)amino]-6-oxo-3-(2-thienyl)-1,6-dihydropyridazine-4-carboxylate
(acetyloxy)methyl 1-ethyl-5-(isoquinolin-4-ylamino)-6-oxo-3-(2-thienyl)-1,6-dihydropyridazine-4-carboxylate
[(isopropoxycarbonyl)oxy]methyl 1-ethyl-5-(isoquinolin-4-ylamino)-6-oxo-3-(2-thienyl)-1,6-dihydropyridazine-4-carboxylate
{[(cyclohexyloxy)carbonyl]oxy}methyl 1-ethyl-5-(isoquinolin-4-ylamino)-6-oxo-3-(2-thienyl)-1,6-dihydropyridazine-4-carboxylate
{[(cyclohexyloxy)carbonyl]oxy}methyl 1-ethyl-6-oxo-5-(pyridin-3-ylamino)-3-(3-thienyl)-1,6-dihydropyridazine-4-carboxylate
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 1
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 2
chloromethyl 1-ethyl-5-[(4-methylpyridin-3-yl)amino]-6-oxo-3-phenyl-1,6-dihydropyridazine-4-carboxylate
(propionyloxy)methyl 1-ethyl-5-[(4-methylpyridin-3-yl)amino]-6-oxo-3-phenyl-1,6-dihydropyridazine-4-carboxylate
{[(1-ethylpropoxy)carbonyl]oxy}methyl 1-ethyl-3-(4-fluorophenyl)-6-oxo-5-(pyridin-3-ylamino)-1,6-dihydropyridazine-4-carboxylate
{[(cyclohexyloxy)carbonyl]oxy}methyl 1-ethyl-3-(4-fluorophenyl)-6-oxo-5-(pyridin-3-ylamino)-1,6-dihydropyridazine-4-carboxylate
chloromethyl 1-ethyl-5-(isoquinolin-4-ylamino)-6-oxo-3-phenyl-1,6-dihydropyridazine-4-carboxylate
(propionyloxy)methyl 1-ethyl-6-oxo-3-phenyl-5-(pyridin-3-ylamino)-1,6-dihydropyridazine-4-carboxylate
(propionyloxy)methyl 1-ethyl-5-(isoquinolin-4-ylamino)-6-oxo-3-phenyl-1,6-dihydropyridazine-4-carboxylate
(pentanoyloxy)methyl 1-ethyl-6-oxo-3-phenyl-5-(pyridin-3-ylamino)-1,6-dihydropyridazine-4-carboxylate
2-oxo-1,3-dioxolan-4-yl 1-ethyl-6-oxo-3-phenyl-5-(pyridin-3-ylamino)-1,6-dihydropyridazine-4-carboxylate
fluoromethyl 1-ethyl-6-oxo-3-phenyl-5-(pyridin-3-ylamino)-1,6-dihydropyridazine-4-carboxylate
1-{[(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 1
1-{[(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 2
   and pharmaceutically acceptable salts thereof.

Of outstanding interest are:
[(2,2-dimethylpropanoyl)oxy]methyl 1-ethyl-6-oxo-3-phenyl-5-(isoquinolin-4-ylamino)-1,6-dihydropyridazine-4-carboxylate
[(2,2-dimethylpropanoyl)oxy]methyl 1-ethyl-6-oxo-3-phenyl-5-(pyridin-3-ylamino)-1,6-dihydropyridazine-4-carboxylate
2-(benzyloxy)-2-oxoethyl 1-ethyl-6-oxo-3-phenyl-5-(pyridin-3-ylamino)-1,6-dihydropyridazine-4-carboxylate
2-(benzyloxy)-2-oxoethyl 1-ethyl-5-[(4-methylpyridin-3-yl)amino]-6-oxo-3-phenyl-1,6-dihydropyridazine-4-carboxylate
(butyryloxy)methyl 1-ethyl-6-oxo-3-phenyl-5-(pyridin-3-ylamino)-1,6-dihydropyridazine-4-carboxylate
3-oxo-1,3-dihydro-2-benzofuran-1-yl 1-ethyl-6-oxo-3-phenyl-5-(pyridin-3-ylamino)-1,6-dihydropyridazine-4-carboxylate
(acetyloxy)methyl 1-ethyl-6-oxo-3-phenyl-5-(pyridin-3-ylamino)-1,6-dihydropyridazine-4-carboxylate
1-(acetyloxy)ethyl 1-ethyl-6-oxo-3-phenyl-5-(pyridin-3-ylamino)-1,6-dihydropyridazine-4-carboxylate
[(2,2-dimethylpropanoyl)oxy]methyl 1-ethyl-3-(3-methylphenyl)-6-oxo-5-(pyridin-3-ylamino)-1,6-dihydropyridazine-4-carboxylate
1-(acetyloxy)-1-methylethyl 1-ethyl-6-oxo-3-phenyl-5-(pyridin-3-ylamino)-1,6-dihydropyridazine-4-carboxylate
[(2,2-dimethylpropanoyl)oxy]methyl 1-ethyl-3-(3-methylphenyl)-6-oxo-5-(pyridin-3-ylamino)-1,6-dihydropyridazine-4-carboxylate
[(2,2-dimethylpropanoyl)oxy]methyl 1-ethyl-3-(4-methylphenyl)-5-[(4-methylpyridin-3-yl)amino]-6-oxo-1,6-dihydropyridazine-4-carboxylate
[(2,2-dimethylpropanoyl)oxy]methyl 1-ethyl-6-oxo-3-phenyl-5-(thieno[2,3-b]pyridin-3-ylamino)-1,6-dihydropyridazine-4-carboxylate
1-[(isopropoxycarbonyl)oxy]ethyl 1-ethyl-6-oxo-3-phenyl-5-(pyridin-3-ylamino)-1,6-dihydropyridazine-4-carboxylate
2-(benzyloxy)-2-oxoethyl 1-ethyl-6-oxo-5-(pyridin-3-ylamino)-3-(2-thienyl)-1,6-dihydropyridazine-4-carboxylate
2-ethoxy-2-oxoethyl 1-ethyl-6-oxo-5-(pyridin-3-ylamino)-3-(2-thienyl)-1,6-dihydropyridazine-4-carboxylate
2-(acetyloxy)ethyl 1-ethyl-5-(isoquinolin-4-ylamino)-6-oxo-3-(3-thienyl)-1,6-dihydropyridazine-4-carboxylate
2-(acetyloxy)ethyl 1-ethyl-5-(isoquinolin-4-ylamino)-6-oxo-3-(2-thienyl)-1,6-dihydropyridazine-4-carboxylate
[(2,2-dimethylpropanoyl)oxy]methyl 1-ethyl-5-(isoquinolin-4-ylamino)-3-(4-methylphenyl)-6-oxo-1,6-dihydropyridazine-4-carboxylate
1-[(isopropoxycarbonyl)oxy]ethyl 1-ethyl-5-[(4-methylpyridin-3-yl)amino]-6-oxo-3-phenyl-1,6-dihydropyridazine-4-carboxylate
2-ethoxy-2-oxoethyl 1-ethyl-5-(isoquinolin-4-ylamino)-6-oxo-3-(2-thienyl)-1,6-dihydropyridazine-4-carboxylate
2-(benzyloxy)-2-oxoethyl 1-ethyl-5-(isoquinolin-4-ylamino)-6-oxo-3-(2-thienyl)-1,6-dihydropyridazine-4-carboxylate
2-ethoxy-2-oxoethyl 1-ethyl-5-(isoquinolin-4-ylamino)-6-oxo-3-(3-thienyl)-1,6-dihydropyridazine-4-carboxylate
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate
1-[(ethoxycarbonyl)oxy]ethyl 1-ethyl-5-[(4-methylpyridin-3-yl)amino]-6-oxo-3-phenyl-1,6-dihydropyridazine-4-carboxylate
1-(acetyloxy)ethyl 1-ethyl-5-(isoquinolin-4-ylamino)-6-oxo-3-phenyl-1,6-dihydropyridazine-4-carboxylate
1-[(ethoxycarbonyl)oxy]ethyl 1-ethyl-6-oxo-3-phenyl-5-(pyridin-3-ylamino)-1,6-dihydropyridazine-4-carboxylate
1-[(isopropoxycarbonyl)oxy]ethyl 1-ethyl-5-(isoquinolin-4-ylamino)-6-oxo-3-phenyl-1,6-dihydropyridazine-4-carboxylate
1-{[(cyclohexyloxy)carbonyl]oxy}ethyl 1-ethyl-5-(isoquinolin-4-ylamino)-6-oxo-3-phenyl-1,6-dihydropyridazine-4-carboxylate
(isobutyryloxy)methyl 1-ethyl-5-(isoquinolin-4-ylamino)-6-oxo-3-phenyl-1,6-dihydropyridazine-4-carboxylate
chloromethyl 1-ethyl-6-oxo-3-phenyl-5-(pyridin-3-ylamino)-1,6-dihydropyridazine-4-carboxylate
{[(cyclohexyloxy)carbonyl]oxy}methyl 1-ethyl-5-(isoquinolin-4-ylamino)-6-oxo-3-phenyl-1,6-dihydropyridazine-4-carboxylate
{[(cyclohexyloxy)carbonyl]oxy}methyl 1-ethyl-6-oxo-3-phenyl-5-(pyridin-3-ylamino)-1,6-dihydropyridazine-4-carboxylate
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 1
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 2
1-{([(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 1
1-{[(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 2
   and pharmaceutically acceptable salts thereof.

According to another embodiment the present invention covers pharmaceutical compositions comprising one or more of the compounds of formula (I), as hereinabove described, in admixture with pharmaceutically acceptable diluents or carriers.

In still another embodiment the present invention covers a combination product comprising (i) a compound of formula (I), as hereinabove described, and (ii) another compound selected from (a) steroids, (b) immunosuppressive agents, (c) T-cell receptor blockers, (d) antiinflammatory drugs, (e) β2-adrenergic agonists and (f) antagonists of M3 muscarinic receptors; for simultaneous, separate or sequential use in the treatment of the human or animal body.

According to still another embodiment of the present invention is directed to the use of a compound of formula (I), as hereinabove described, in the manufacture of a medicament for the treatment or prevention of a pathological condition or disease susceptible to amelioration by inhibition of phosphodiesterase 4. It is a preferred embodiment to use the compound of formula (I) in the manufacture of a medicament for use in the treatment or prevention of a disorder which is asthma, chronic obstructive pulmonary disease, rheumatoid arthritis, atopic dermatitis, psoriasis or irritable bowel disease.

According to still another embodiment the present invention covers a method for treating a subject afflicted with a pathological condition or disease susceptible to amelioration by inhibition of phosphodiesterase 4, which method comprises administering to the said subject an effective amount of a compound of formula (I), as hereinabove described. In a preferred embodiment the method is used for treating a subject afflicted with a pathological condition or disease which is asthma, chronic obstructive pulmonary disease, rheumatoid arthritis, atopic dermatitis, psoriasis or irritable bowel disease.

The compounds of the present invention may be prepared by one of the processes described below.

Compounds of formula (I) may be obtained from the intermediates of formula (IIa) or (IIb) through the reaction paths shown in Scheme 1.

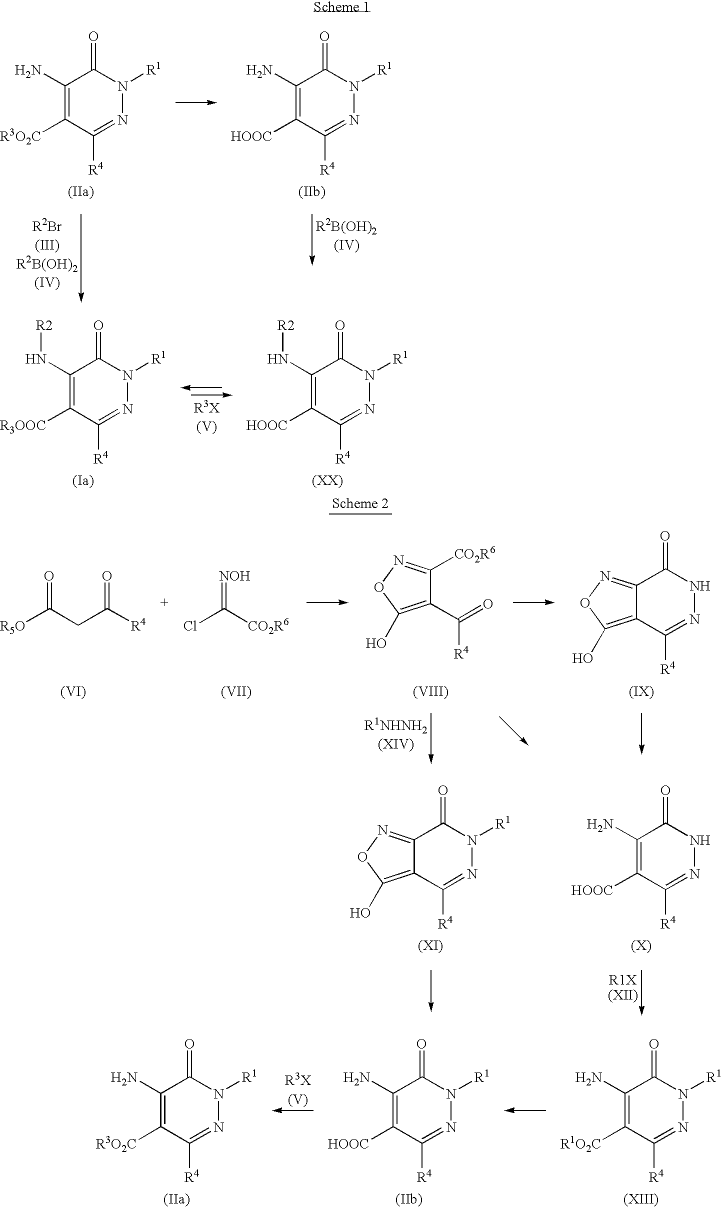

Condensation of a 5-amino-6-oxo-1,6-dihydropyridazine-4-carboxylate of formula (IIa) wherein R¹, R³ and R⁴ are as hereinbefore defined, with an heteroaryl bromide (III), wherein R² is as hereinbefore defined, gives the final compound (Ia). The reaction is carried out in the presence of a copper salt such as cuprous iodide in the presence of an organic base, preferably a diamine base such as N,N′-dimethylethylenediamine and of an inorganic base such as potassium carbonate in an inert solvent such as toluene, dioxane or dimethylformamide, at a temperature from −20° C. to the boiling point of the solvent.

Hydrolysis of 5-amino-6-oxo-1,6-dihydropyridazine-4-carboxylates of formula (IIa) yields 5-amino-6-oxo-1,6-dihydropyridazine-4-carboxylic acids (IIb), wherein R¹ and R⁴ are as hereinbefore defined.

Alternatively, condensation of 5-amino-6-oxo-1,6-dihydropyridazine-4-carboxylates of formula (IIa) wherein R¹, R³ and R⁴ are as hereinbefore defined, with boronic acids (IV), wherein R² is as hereinbefore defined, gives compounds (Ia). The same reaction using compound (IIb) wherein R¹, R³ and R⁴ are as hereinbefore defined yields compound (XX). The reaction is carried out in the presence of a copper salt such as cupric acetate in the presence of an organic base, preferably an amine base such as triethylamine, in an inert solvent such as dioxane, methylene chloride or tetrahydrofuran, at a temperature from −20° C. to the boiling point of the solvent.

Hydrolysis of 5-heteroarylamino-6-oxo-1,6-dihydropyridazine-4-carboxylates of formula (Ia) yields 5-heteroaylamino-6-oxo-1,6-dihydropyridazine-4-carboxylic acids (XX), wherein R¹ and R⁴ are as hereinbefore defined.

Alternatively, reaction of 5-heteroaylamino-6-oxo-1,6-dihydropyridazine-4-carboxylates of formula (XX) with an alkylating agent of formula (V), wherein R³ is as hereinbefore defined and X is a leaving group such as a chlorine or a bromine atom or a methanesulfonate, p-toluenesulfonate or a benzenesulfonate, gives the final product (Ia). The reaction is carried out in the presence of an organic base, preferably an amine base such as diisopropylethylamine or an inorganic base such as potassium carbonate in an inert solvent such as DMF, acetone or tetrahydrofuran, at a temperature from −20° C. to the boiling point of the solvent.

5-Amino-6-oxo-1,6-dihydropyridazine-4-carboxylates of formula (II) may be obtained as shown in Scheme 2.

Reaction of 1,3-dicarbonylic compounds of general formula (VI), wherein R⁴ is as hereinabove defined and R⁵ is a C₁ to C₆ alkyl group, and 2-chloro-2-(hydroxyimino)acetate derivatives of formula (VII), wherein R⁶ is a C₁ to C₆ alkyl group, following methods known per se, e.g. G. Renzi et al., Gazz. Chim. Ital. 1965, 95, 1478, gives isoxazole derivatives of formula (VIII).

Isoxazole derivatives of formula (VIII) are condensed with hydrazine, by methods known per se, e.g. G. Renzi et al., Gazz. Chim. Ital. 1965, 95, 1478 and V. Dal Piaz et al. Heterocycles 1991, 32, 1173, to give isoxazolo[3,4-d]pyridazin-7(6H)-ones of formula (IX) wherein R⁴ is as hereinbefore defined.

Isoxazolo[3,4-d]pyridazin-7-ones (IX), wherein R⁴ is as hereinbefore defined, are reduced to yield 5-amino-6-oxo-1,6-dihydro-pyridazine-4-carboxylic acids (X). The reaction may be performed with hydrazine in a solvent such as ethanol at its boiling point. This reaction may also be performed by hydrogenation using for example hydrogen in the presence of a catalyst by methods known per se, e.g. V. Dal Piaz et al. Heterocycles, 1991, 32, 1173. Alternatively, the reaction may be accomplished by transfer hydrogenation using an organic hydrogen donor and a transfer agent, such as ammonium formate or hydrazine by methods known per se, e.g. V. Dal Piaz et al. Heterocycles, 1991, 32, 1173.

Alternatively 5-amino-6-oxo-1,6-dihydro-pyridazine-4-carboxylic acids (X) can be directly obtained from isoxazolo derivatives (VIII) by treatment with hydrazine. The reaction is carried out in an inert solvent such as ethanol at a temperature from −20° C. to the boiling point of the solvent.

Subsequent reaction of 5-amino-6-oxo-1,6-dihydropyridazine-4-carboxylic acids of formula (X) with an alkylating agent of formula (XII), wherein R′ is as hereinbefore defined and X is a leaving group such as a chlorine or a bromine atom or a methanesulfonate, p-toluenesulfonate or a benzenesulfonate group, by methods known per se, e.g. V. Dal Piaz et al. Drug Des. Discovery 1996, 14, 53, gives 5-amino-6-oxo-1,6-dihydropyridazine-4-carboxylates of formula (XIII).

Hydrolysis of 5-amino-6-oxo-1,6-dihydropyridazine-4-carboxylates of formula (XIII) yields 5-amino-6-oxo-1,6-dihydropyridazine-4-carboxylic acids (IIb), wherein R¹ and R⁵ are as hereinbefore defined.

Reaction of 5-amino-6-oxo-1,6-dihydropyridazine-4-carboxylic acids of formula (IIb), with an alkylating agent of formula (V), wherein R³ is as hereinbefore defined and X is a leaving group such as a chlorine or a bromine atom or a methanesulfonate, p-toluenesulfonate or a benzenesulfonate, gives 5-amino-6-oxo-1,6-dihydropyridazine-4-carboxylates (IIa), wherein R¹, R³ and R⁴ are as hereinbefore defined. The reaction is carried out in the presence of an organic base, preferably an amine base such as diisopropylethylamine or an inorganic base such as potassium carbonate in an inert solvent such as DMF, acetone or tetrahydrofuran, at a temperature from −20° C. to the boiling point of the solvent.

Alternatively, 5-amino-6-oxo-1,6-dihydropyridazine-4-carboxylic acids (IIb), wherein R¹ and R⁴ are as hereinbefore defined may be obtained from isoxazoles (VIII) where R⁴ and R⁶ are as hereinbefore defined by condensation with a hydrazine of formula (XIV), where R¹ is as hereinbefore defined, by methods known per se, e.g. G. Renzi et al., Gazz. Chim. Ital. 1965, 95, 1478, to give isoxazolo[3,4-d]pyridazin-7(6H)-ones of formula (XI) wherein R¹ and R⁴ are as hereinbefore defined. Subsequent hydrogenation using for example hydrogen in the presence of a catalyst by methods known per se, e.g. V. Dal Piaz et al. Heterocycles, 1991, 32, 1173 yields the 5-amino-6-oxo-1,6-dihydropyridazine-4-carboxylic acids (IIb), wherein R¹ and R⁴ are as hereinbefore defined. Alternatively, the reaction may be accomplished by transfer hydrogenation using an organic hydrogen donor and a transfer agent, such as ammonium formate or hydrazine by methods known per se, e.g. V. Dal Piaz et al. Heterocycles, 1991, 32, 1173.

Alternatively, 5-amino-6-oxo-1,6-dihydropyridazine-4-carboxylates of formula (II) may be obtained as shown in Scheme 3.

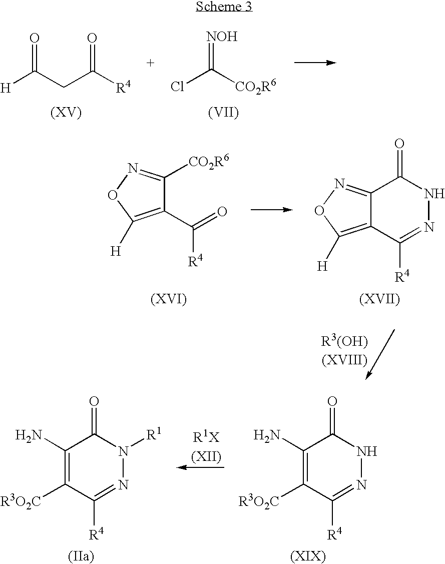

Reaction of 1,3-dicarbonylic compounds of general formula (XV), wherein R⁴ is as hereinabove defined and 2-chloro-2-(hydroxyimino)acetate derivatives of formula (VII), wherein R⁶ is a C₁ to C₆ alkyl group, following methods known per se, e.g. G. Renzi et al., Gazz. Chim. Ital. 1965, 95, 1478, gives isoxazole derivatives of formula (XVI).

Isoxazole derivatives of formula (XVI) are condensed with hydrazine, by methods known per se, e.g. G. Renzi et al., Gazz. Chim. Ital. 1965, 95, 1478 and V. Dal Piaz et al. Heterocycles 1991, 32, 1173, to give isoxazolo[3,4-d]pyridazin-7(6H)-ones of formula (XVII) wherein R⁴ is as hereinbefore defined.

Compounds (XVII) are reacted with alcohols of general formula (XVIII), wherein R³ is as hereinbefore defined, to give 5-amino-6-oxo-1,6-dihydropyridazine-4-carboxylates of formula (XIX). The reaction is carried out in the presence of an organic base, preferably an amine base such as triethylamine or piperidine, at a temperature from room temperature to the boiling point of the alcohol.

Subsequent reaction of 5-amino-6-oxo-1,6-dihydropyridazine-4-carboxylates of formula (XIX) with an alkylating agent of formula (XII), wherein R′ is as hereinbefore defined and X is a leaving group such as a chlorine or a bromine atom or a methanesulfonate, p-toluenesulfonate or a benzenesulfonate group, by methods known per se, e.g. V. Dal Piaz et al. Drug Des. Discovery 1996, 14, 53, gives 5-amino-6-oxo-1,6-dihydropyridazine-4-carboxylates of formula (IIa).

When the defined groups R¹ to R⁵ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3^{rd} Edition, John Wiley & Sons (1999). It may be that deprotection will form the last step in the synthesis of compounds of formula (I).

The compounds of formulae (III), (IV), (V), (VI), (VII) and (XV) are known compounds or can be prepared by analogy with known methods.

### Experimental

### Plasma Stability Assay

For plasma stability assays, compounds in acetonitrile or dimethylsufoxide solutions are added in duplicate to 1 mL plasma pre-warmed at 37° C. at a final concentration of 1 μg/mL (less than 1% organic solvent added). Just after the addition of the compounds and mixing (t=0 h), 100 μL samples are collected and transferred to tubes containing 300 μL of 0.5% trifluoro acetic acid in acetonitrile in an ice bath in order to stop the reaction. Samples are kept in a water bath at 37° C. during the assay. At different time intervals (i.e. t=0.5, 1, 3 and 24 h) samples are collected and reaction stopped as described previously. The aliquots are centrifuged at 4000 rpm for 10 minutes, 100 μL of supernatant diluted with 100 μL Milli-Q water and 5 μL injected in a HPLC/MS system. Both the parent compound and the possible by-products are monitored. The stability is calculated by comparing the compound response obtained with the response a time 0 h.

### Pharmacological Activity

### PDE4 Assay Procedure

Compounds to be tested were resuspended in DMSO at a stock concentration of 1 mM. The compounds were tested at different concentrations varying from 10 μM to 10 pM to calculate an IC₅₀. These dilutions were done in 96-well plates. In some cases, plates containing diluted compounds were frozen before being assayed. In these cases, the plates were thawed at room temperature and stirred for 15 minutes.

Ten microliters of the diluted compounds were poured into a “low binding” assay plate. Eighty microliters of reaction mixture containing 50 mM Tris pH 7.5, 8.3 mM MgCl₂, 1.7 mM EGTA, and 15 nM [3H]-cAMP were added to each well. The reaction was initiated by adding ten microliters of a solution containing PDE4. The plate was then incubated under stirring for 1 hour at room temperature. After incubation the reaction was stopped with 50 microlitres of SPA beads, and the reaction was allowed to incubate for another 20 minutes at room temperature before measuring radioactivity using standard instrumentation.

The reaction mixture was prepared by adding 90 ml of H₂O to 10 ml of 10× assay buffer (500 mM Tris pH 7.5, 83 mM MgCl₂, 17 mM EGTA), and 40 microlitres 1 μCi/μL [3H]-cAMP. SPA beads solution was prepared by adding 500 mg to 28 ml H₂O for a final concentration of 20 mg/ml beads and 18 mM zinc sulphate.

The results are shown in Table 1.

| | HPDE4B or |
|---|---|
| | IC₅₀ PDE4 |
| No | (nM) |
| 16 | 0.22 |
| 17 | 3.6 |
| 18 | 2.1 |
| 19 | 11 |
| 23 | 0.94 |
| 26 | 2.3 |
| 27 | 9.1 |
| 31 | 0.07 |
| 35 | 2.2 |
| 68 | 5.7 |
| 76 | 4.6 |
| 94 | 0.5 |
| 122 | 3.4 |
| 132 | 0.3 |
| 133 | 0.8 |
| 142 | 1.2 |
| 164 | 0.1 |

It can be seen from Table 1 that the compounds of formula (I) are potent inhibitors of phosphodiesterase 4 (PDE 4). Preferred pyridazin-3(2H)-one derivatives of the invention possess an IC₅₀ value for the inhibition of PDE4 (determined as defined above) of less than 100 nM, preferably less than 50 nM and most preferably less than 30 nM. The compounds are also capable of blocking the production of some pro-inflammatory cytokines such as, for example, TNFα.

Thus, they can be used in the treatment of allergic, inflammatory and immunological diseases, as well as those diseases or conditions where the blockade of pro-inflammatory cytokines or the selective inhibition of PDE 4 could be of benefit. These disease states include asthma, chronic obstructive pulmonary disease, allergic rhinitis, rheumatoid arthritis, osteoarthritis, osteoporosis, bone-formation disorders, glomerulonephritis, multiple sclerosis, ankylosing spondylitis, Graves ophtalmopathy, myasthenia gravis, diabetes insipidus, graft rejection, gastrointestinal disorders such as irritable bowel disease, ulcerative colitis or Crohn disease, septic shock, adult distress respiratory syndrome, and skin diseases such as atopic dermatitis, contact dermatitis, acute dermatomyositis and psoriasis. They can also be used as improvers of cerebrovascular function as well as in the treatment of other CNS related diseases such as dementia, Alzheimer's disease, depression, and as nootropic agents.

The compounds of the present invention show a short half life in plasma, which is preferably shorter than 5 hours, more preferably shorter than 3 hours and most preferably shorter than 1 hour. The free acid derivatives originating from the hydrolisys of the group —COOR³ of the compounds of the present invention have an IC₅₀ value for the inhibition of PDE4 which is several times higher than the IC₅₀ value of the non-hydrolised compounds.

Consequently the pyridazin-3(2H)-one derivative of the invention can be administered to a subject in need thereof at relatively high doses without causing undesirable systemic effects as a result of both their short half lifes in plasma and the reduced PDE4 inhibition capacity of the their hydrolisates.

The compounds of the present invention are also of benefit when administered in combination with other drugs such as steroids and immunosuppressive agents, such as cyclosporin A, rapamycin, T-cell receptor blockers, β2-adrenergic agonists or antagonists of M3 muscarinic receptors. In this case the administration of the compounds allows a reduction of the dosage of the other drugs, thus preventing the appearance of the undesired side effects associated with both steroids and immunosuppressants.

Like other PDE4 inhibitors (see references above) the compounds of the invention can also be used for blocking, after preventive and/or curative treatment, the erosive and ulcerogenic effects induced by a variety of etiological agents, such as antiinflammatory drugs (steroidal or non-steroidal antiinflammatory agents), stress, ammonia, ethanol and concentrated acids.

They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease.

They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the pyridazin-3(2H)-one derivatives of the invention and pharmaceutically acceptable salts thereof, and pharmaceutical compositions comprising such compound and/or salts thereof, may be used in a method of treatment or prevention of disorders of the human body susceptible to amelioration by inhibition of phosphodiesterase 4 which comprises administering to a patient requiring such treatment an effective amount of a pyridazin-3(2H)-one derivative of the invention.

Accordingly, another embodiment of the invention is the use of the compounds of formula (I) in the manufacture of a medicament for treatment or prevention of pathological conditions, diseases and disorders known to be susceptible of amelioration by inhibition of PDE4, as well as a method for treating a subject afflicted with a pathological condition or disease susceptible to amelioration by inhibition of PDE4, which comprises administering to said subject an effective amount of a compound of formula (I).

The present invention also provides pharmaceutical compositions which comprise, as an active ingredient, at least a pyridazin-3(2H)-one derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight, of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Compositions for topical administration may take the form of ointments, creams or lotions, all containing the compound of the invention; such preparations may be made by methods well-known in the art.

Effective doses are normally in the range of 10-600 mg of active ingredient per day. Daily dosage may be administered in one or more treatments, preferably from 1 to 4 treatments, per day.

The present invention will be further illustrated by the following examples. The examples are given by way of illustration only and are not to be construed as a limiting.

The syntheses of the compounds of the invention and of the intermediates for use therein are illustrated by the following Examples (including Preparation Examples (Preparations 1 to 33)) which do not limit the scope of the invention in any way.

¹H Nuclear Magnetic Resonance Spectra were recorded on a Varian Gemini 300 spectrometer.

Low Resolution Mass Spectra (m/z) were recorded on a Micromass ZMD mass spectrometer using ESI ionization.

Melting points were recorded using a Perkin Elmer DSC-7 apparatus.

The chromatographic separations (standard method) were obtained using a Waters 2690 system equipped with a Symmetry C18 (2.1×10 mm, 3.5 μm) column. The mobile phase was formic acid (0.4 mL), ammonia (0.1 mL), methanol (500 mL) and acetonitrile (500 mL) (B) and formic acid (0.46 mL), ammonia (0.115 mL) and water (1000 mL) (A): initially from 0% to 95% of B in 18 min, and then 4 min. with 95% of B. The reequilibration time between two injections was 5 min. The flow rate was 0.4 mL/min. The injection volume was 5 microliter. Diode array chromatograms were collected at 210 nM.

The chromatographic separations (method B) were obtained using a Waters 2690 system equipped with a Symmetry C18 (2.1×10 mm, 3.5 μm) column. The mobile phase was formic acid (0.4 mL), ammonia (0.1 mL), methanol (500 mL) and acetonitrile (500 mL) (B) and formic acid (0.46 mL), ammonia (0.115 mL) and water (1000 mL) (A): initially from 0% to 95% of B in 26 min, and then 4 min. with 95% of B. The reequilibration time between two injections was 5 min. The flow rate was 0.4 mL/min. The injection volume was 5 microliter. Diode array chromatograms were collected at 210 nM.

### PREPARATION EXAMPLES

### Preparation 1

### Ethyl 4-benzoyl-5-hydroxyisoxazole-3-carboxylate

To a cooled and stirred solution of sodium ethoxide, obtained from sodium (2.3 g, 0.1 mol) and anhydrous EtOH (60 ml), a solution of ethyl benzoylacetate (9.6 g, 0.05 mol) in the same solvent (5 ml) was slowly added. A solution of ethylcloro(hydroximino)acetate (7.55 g, 0.05 mol) in anhydrous EtOH (10 ml) was added in a dropwise manner (over 1 h period). The mixture was neutralized with 6N HCl and the alcoholic layer was evaporated. After dilution with cold water (150-200 ml), the suspension was extracted with ethyl ether and the aqueous layer was acidified with 6N HCl to afford the product which was recovered by filtration (45% yield).

δ(DMSO-d₆): 1.25 (t, 3H), 4.15 (q, 2H), 7.50 (m, 3H), 7.80 (m, 2H), 10.80 (s, 1H).

### Preparation 2

### 4-Phenyl-1,6-dihydro-isoxazolo[3,4-d]pyridazine-3,7-dione

To a stirred solution of the title product of Preparation 1 (15.0 g, 0.057 mol) in dry ethanol (150 ml), hydrazine hydrate (10.2 ml, 0.203 mol) was added dropwise and the resulting mixture was stirred at r.t. overnight. The solid thus formed was filtered and washed with cold ethanol and ethyl ether to yield 13.6 g of the title product (92% yield).

δ(DMSO-d₆): 7.37 (m, 3H), 7.82 (m, 2H).

### Preparation 3

### 5-Amino-6-oxo-3-phenyl-1,6-dihydro-pyridazine-4-carboxylic acid

To a stirred solution of the title product of Preparation 2 (6.0 g, 0.026 mol) in dry ethanol (80 ml), hydrazine hydrate (5 ml, 0.10 mol) was added dropwise and the resulting mixture was refluxed overnight. Then it was let to cool down and the solid thus formed was filtered and washed with cold ethanol and ethyl ether. 5.0 g of the title product were obtained (83% yield)

δ(DMSO-d₆): 6.62 (bs, 2H), 7.27 (m, 3H), 7.37 (m, 2H).

### Preparation 4

### 5-Amino-1-ethyl-6-oxo-3-phenyl-1,6-dihydro-pyridazine-4-carboxylic acid ethyl ester

To a stirred solution of the title product of Preparation 3 (13.3 g, 0.057 mol) in dry DMF (160 ml), potassium carbonate (31.6 g, 0.228 mol) was added portionwise and the resulting mixture was stirred at 70° C. for 1 h. Then it was let to cool down and the ethyl bromide (17.1 ml, 0.229 mol) in dry DMF (30 ml) was added dropwise during 15 min. The final mixture was stirred at 70° C. for 6 h and then the solvent was removed under reduced pressure. The crude thus obtained was suspended in ice-water and extracted with dichloromethane twice. The organic layer was then washed with saturated NaHCO3 solution, water and brine. It was dried and solvent was removed under reduced pressure to yield the title product (75% yield).

δ(DMSO-d₆): 0.78 (t, 3H), 1.25 (t, 3H), 3.90 (q, 2H), 4.10 (q, 2H), 7.28 (m, 2H), 7.37 (m, 3H), 7.55 (s, 2H).

### Preparation 5

### 5-Amino-1-ethyl-6-oxo-3-phenyl-1,6-dihydro-pyridazine-4-carboxylic acid

To a stirred suspension of the title product of Preparation 4 (6.1 g, 0.021 mol) in methanol (78 ml), 2N NaOH (31.6 ml, 0.63 mol) was added dropwise and the resulting mixture was stirred at rt overnight and then at 80° C. for 1 h. Then it was let to cool down and half of the methanol was removed under reduced pressure. It was neutralized to pH 6-7 with HCl 1N. The solid thus obtained was filtered, washed with ethyl ether and dried to yield the title product (71%).

δ(DMSO-d6): 1.24 (t, 3H), 4.05 (q, 2H), 7.33 (m, 3H), 7.42 (m, 2H), 12.95 (s, 1H).

### Preparation 6

### Benzyloxycarbonylmethyl 5-amino-1-ethyl-6-oxo-3-phenyl-1,6-dihydro-pyridazine-4-carboxylate

To a stirred mixture of the title product of Preparation 5 (1.0 g, 3.86 mmol) in dry DMF (40 ml), potassium carbonate (0.64 g, 4.62 mmol) was added portionwise and the resulting mixture was stirred for a while. Then, benzyl bromoacetate (0.74 ml, 4.62 mmol) was added dropwise and the final mixture was stirred at rt overnight. The reaction crude was poured onto water and extracted with ethyl ether. The combined organic layers were washed with brine and dried. Solvent was then removed under reduced pressure to yield the title product (1.48 g, 98% yield).

LRMS: m/Z 408 (M+1)⁺.

δ(CDCl₃): 1.38 (t, 3H), 4.26 (q, 2H), 4.45 (s, 2H), 5.15 (s, 2H), 7.30 (m, 12H).

### Preparation 7

### Ethyl 4-(3-methylbenzoyl)-5-oxo-2,5-dihydro-isoxazole-3-carboxylate

Obtained as a solid (40%) from ethyl 3-oxo-3-m-tolyl-propionate following the experimental procedure of Preparation 1.

LRMS: m/Z 276 (M+1)⁺.

δ(DMSO-d₆): 1.18 (t, 3H), 2.25 (s, 3H), 4.10 (q, 2H), 7.25 (m, 2H), 7.50 (m, 3H).

### Preparation 8

### 4-m-Tolyl-1,6-dihydro-isoxazolo[3,4-d]pyridazine-3,7-dione

Obtained as a solid (64%) from the title compound of Preparation 7 following the experimental procedure of Preparation 2.

LRMS: m/Z 244 (M+1)⁺.

δ(DMSO-d₆): 2.25 (s, 3H), 7.25 (m, 2H), 7.60 (m, 2H), 11.5 (s, 1H).

### Preparation 9

### 5-Amino-6-oxo-3-m-tolyl-1,6-dihydro-pyridazine-4-carboxylic acid

Obtained as a solid (35%) from the title compound of Preparation 8 following the experimental procedure of Preparation 3.

LRMS: m/Z 244 (M−1)⁺.

δ(DMSO-d₆): 2.45 (s, 3H), 6.95 (bs, 2H), 7.30 (m, 4H).

### Preparation 10

### 5-Amino-1-ethyl-6-oxo-3-m-tolyl-1,6-dihydro-pyridazine-4-carboxylic acid ethyl ester

Obtained as a solid (90%) from the title compound of Preparation 9 following the experimental procedure of Preparation 4.

LRMS: m/Z 302 (M+1)⁺.

δ(CDCl₃): 0.79 (t, 3H), 1.38 (t, 3H), 2.38 (s, 3H), 3.92 (q, 2H), 4.22 (q, 2H), 7.20 (m, 4H).

### Preparation 11

### Ethyl 4-(3-fluorobenzoyl)-5-oxo-2,5-dihydro-isoxazole-3-carboxylate

Obtained as a solid (65%) from the title compound 3-(3-fluorophenyl)-3-oxopropionic acid ethyl ester following the experimental procedure of Preparation 39.

LRMS: m/Z 279 (M+1)⁺.

δ(CDCl₃): 1.00 (t, 3H), 3.82 (q, 2H), 7.25 (m, 4H).

### Preparation 12

### 5-Amino-3-(3-fluoro-phenyl)-6-oxo-1,6-dihydro-pyridazine-4-carboxylic acid

To a stirred solution of the title product of Preparation 11 (2.45 g, 8.8 mmol) in dry ethanol (25 ml), hydrazine hydrate (2.5 ml, 53 mmol) was added dropwise and the resulting mixture was refluxed overnight. Then it was let to cool down and the solid thus formed was filtered and washed with cold ethanol and ethyl ether. 1.7 g of the title product were obtained (77% yield)

LRMS: m/Z 250 (M+1)⁺.

Retention time: 5.3 min.

### Preparation 13

### 5-Amino-1-ethyl-3-(3-fluoro-phenyl)-6-oxo-1,6-dihydro-pyridazine-4-carboxylic acid ethyl ester

Obtained as a solid (22%) from the title compound of Preparation 12 following the experimental procedure of Preparation 4.

LRMS: m/Z 306 (M+1)⁺.

δ(CDCl₃): 0.82 (t, 3H), 1.19 (t, 3H), 3.98 (q, 2H), 4.22 (q, 2H), 7.10 (m, 3H), 7.38 (m, 1H).

### Preparation 14

### Ethyl 4-(4-fluorobenzoyl)-5-oxo-2,5-dihydro-isoxazole-3-carboxylate

Obtained as a solid (62%) from the title compound 3-(4-fluorophenyl)-3-oxopropionic acid ethyl ester following the experimental procedure of Preparation 39.

LRMS: m/Z 279 (M+1)⁺.

δ(DMSO-d₃): 1.18 (t, 3H), 4.17 (q, 2H), 7.17 (t, 2H), 7.82 (m, 2H).

### Preparation 15

### 5-Amino-3-(4-fluorophenyl)-6-oxo-1,6-dihydro-pyridazine-4-carboxylic acid

Obtained as a solid (89%) from the title product of Preparation 14 following the experimental procedure of Preparation 12.

LRMS: m/Z 250 (M+1)⁺.

δ(DMSO-d₃): 7.25 (t, 2H), 7.62 (m, 2H).

### Preparation 16a

### Ethyl 5-amino-1-ethyl-3-(4-fluoro-phenyl)-6-oxo-1,6-dihydro-pyridazine-4-carboxylate

Obtained as a solid (30%) from the title compound of Preparation 15 following the experimental procedure of Preparation 4.

LRMS: m/Z 306 (M+1)⁺.

Retention Time: 8.6 min*. *Chromatographic method B.

δ(CDCl₃): 0.82 (t, 3H), 1.38 (t, 3H), 3.98 (q, 2H), 4.22 (q, 2H), 7.05 (t, 2H), 7.36 (m, 2H).

### Preparation 16b

### Methyl 5-amino-1-ethyl-6-oxo-3-phenyl-1,6-dihydro-pyridazine-4-carboxylate

Obtained as a solid (88%) from the title compound of Preparation 5 and methyl iodide following the experimental procedure of Preparation 6.

LRMS: m/Z 274 (M+1)⁺.

δ(CDCl₃): 1.38 (t, 3H), 3.41 (s, 3H), 4.22 (q, 2H), 7.40 (s, 5H).

### Preparation 17

### Ethyl 3-oxo-3-thiophen-2-ylpropionate

Diethyl carbonate (39.6 ml, 327 mmol) in toluene (20 ml) was heated to 60° C. At this temperature, potassium tert-butoxide (14.3 g, 128 mmol) was portionwise added and, once the addition was over, heated at 65° C. for half an hour. Then the temperature was raised to 75° C. and 2-acethylthiophene (10.0 g, 79 mmol) in toluene (20 ml) was dropwise added. The reaction mixture was heated at 80° C. for 45 min, then allowed to reach room temperature and finally poured into water. After successive extractions with ethyl acetate, the organic phase was dried over sodium sulfate, filtered and evaporated. 14.2 g of a dark oil were obtained as the desired final product (90% yield).

δ(CDCl₃): 1.25 (t, 3H), 3.90 (s, 2H), 4.20 (q, 2H), 7.1 (m, 1H), 7.70 (m, 1H), 7.75 (m, 1H).

### Preparation 18

### Ethyl 5-oxo-4-(thiophen-2-carbonyl)-2,5-dihydroisoxazol-3-carboxylate

Sodium (6.4 g, 0.28 mol) was dissolved at room temperature in ethanol (165 ml). This solution was cooled in an ice-bath and the title product of Preparation 17 (27.5 g, 0.14 mol) in ethanol (20 ml) was dropwise added. After 15 min at 0° C. under stirring, ethyl cloroximido acetate (21.1 g, 0.14 mol) in ethanol (40 ml) was dropwise added. After 1.5 h at 0° C. under stirring, the reaction mixture was allowed to reach room temperature and left overnight under these conditions. Ethanol was removed under reduced pressure and the residue was suspended in water. This reaction mixture was then neutralized with 2N HCl and washed once with Et₂O. The aqueous phase was then acidified with 5N HCl and a yellow solid precipitates, which was filtered and washed with Et₂O. 16.4 g of the desired final product were isolated (44% yield).

δ(CDCl₃): 1.50 (t, 3H), 2.20 (bs, 1H), 4.60 (q, 2H), 7.25 (m, 1H), 7.85 (m, 1H), 9.0 (bs, 1H).

### Preparation 19

### 4-Thiophen-2-yl-1,6-dihydroisoxazolo[3,4-d]pyridazin-3,7-dione

The title product of Preparation 18 was suspended in ethanol (65 ml) and hydrazine monohydrate (4.3 ml, 89.7 mmol) was dropwise added. After 18 h at room temperature under stirring, the yellow solid was filtered (6.4 g) and resuspended in ethanol (65 ml). This mixture was heated under refluxed for 18 h and the solvent evaporated under reduced pressure. The residue was triturated with Et₂O, filtered and dried. 5.6 g of the desired final product were obtained (94% yield).

δ(CDCl₃): 6.60 (bs, 1H), 7.10 (m, 1H), 7.50 (m, 1H), 8.80 (m, 1H), 11.6 (bs, 1H).

### Preparation 20

### 5-Amino-6-oxo-3-thiophen-2-yl-1,6-dihydropyridazin-4-carboxylic acid

The title product of Preparation 19 (12.5 g, 53.3 mmol) was suspended in ethanol (160 ml) and hydrazine monohydrate (9.9 ml, 0.20 mol) was added. After refluxing the mixture for 18 h, the suspended solid was filtered and washed with Et₂O. 11.3 g of the desired final compound were obtained (90% yield).

δ(DMSO-d₆): 6.20 (s, 2H), 7.0 (m, 1H), 7.40 (m, 4H).

### Preparation 21

### Ethyl 5-amino-1-ethyl-6-oxo-3-thiophen-2-yl-1,6-dihydropyridazin-4-carboxylate

The title product of Preparation 20 (11.3 g, 47.8 mmol) was dissolved in dimethylformamide (135 ml) and potassium carbonate (26.4 g, 190.9 mmol) was added. This mixture was heated to 70° C. for 1 h. Then it was cooled again to room temperature and bromoethane (14.3 g, 242.2 mmol) in DMF (25 ml) was dropwise added to the mixture. After heating at 70° C. for 72 h, the reaction mixture was poured into water and extracted repeatedly with Et₂O. This organic phase was washed with 4% NaHCO3, water and brine, dried over magnesium sulfate, filtered and evaporated to dryness. 12.2 g of the desired final compound (87% yield) were obtained as an oil.

δ(CDCl₃): 0.95 (t, 3H), 1.40 (t, 3H), 4.10 (q, 2H), 4.25 (q, 2H), 7.05 (m, 4H), 7.40 (m, 1H).

### Preparation 22

### Ethyl 3-oxo-3-(3-thienyl)propionate

Diethyl carbonate (36.3 ml, 300 mmol) in toluene (18 ml) was heated to 60° C. At this temperature, potassium tert-butoxide (13.0 g, 120 mmol) was portionwise added and, once the addition was over, heated at 65° C. for half an hour. Then the temperature is rawased to 75° C. and 3-acethylthiophene (9.29, 73 mmol) in toluene (18 ml) was dropwise added. The reaction mixture was heated at 80° C. for 90 min, then allowed to reach room temperature and the precipitated solid was filtrated and washed thoroughly with ether. This solid was dissolved in water. After successive extractions with ethyl acetate, the organic phase was washed with brine and dried over sodium sulfate, filtered and evaporated. A dark oil was obtained (12.0 g, 83% yield) as the desired final product.

δ(CDCl₃): 1.25 (t, 3H), 3.90 (s, 2H), 4.20 (q, 2H), 7.35 (m, 1H), 7.55 (m, 1H), 8.10 (m, 1H).

### Preparation 23

### Ethyl 5-oxo-4-(thiophen-3-carbonyl)-2,5-dihydroisoxazol-3-carboxylate

Sodium (2.5 g, 0.11 mol) was dissolved at room temperature in ethanol (65 ml). This solution was cooled in an ice-bath and the title product of Preparation 22 (12.0 g, 6.6 mmol) in ethanol (12 ml) was dropwise added. After 15 min at 0° C. under stirring, ethyl cloroximido acetate (8.4 g, 55.4 mmol) in ethanol (12 ml) was dropwise added. After 1 h at 0° C. under stirring, the reaction mixture was allowed to reach room temperature and left overnight under these conditions. Ethanol was removed under reduced pressure and the residue redissolved in water. This reaction mixture was then neutralized with 2N HCl and washed once with Et₂O. The aqueous phase was then acidified with 5N HCl and extracted with Et₂O. The organic phase was dried with magnesium sulfate, filtered and evaporated under reduced pressure to yield the title product as an oil (9.2 g, 62%).

δ(CDCl₃): 1.50 (t, 3H), 4.55 (q, 2H), 7.35 (m, 2H), 7.75 (m, 1H), 8.85 (bs, 1H).

### Preparation 24

### 4-Thiophen-3-yl-1,6-dihydroisoxazolo[3,4-d]pyridazin-3,7-dione

The title product of Preparation 23 (9.2 g, 34.4 mmol) was suspended in ethanol (90 ml) and hydrazine monohydrate (5.9 ml, 122.1 mmol) was dropwise added. After 48 h at room temperature under stirring, the yellow solid was filtered and washed thoroughly with ethanol and ether. Once dried, 6.21 g of the desired final product were obtained. (77% yield).

δ(CDCl₃): 7.40 (bs, 1H), 7.50 (m, 1H), 7.65 (m, 1H), 9.0 (s, 1H), 11.6 (bs, 1H).

### Preparation 25

### 5-Amino-6-oxo-3-thiophen-3-yl-1,6-dihydropyridazin-4-carboxylic acid

The title product of Preparation 24 (6.2 g, 26.4 mmol) was suspended in ethanol and hydrazine monohydrate (4.9 ml, 100.7 mmol) was added. The resulting mixture was refluxed for 18 h and the solid thus formed was filtered and washed with Et₂O. Once dried, 3.8 g of the desired final solid were obtained. (60%, yield).

δ(DMSO-d₆): 6.60 (s, 2H), 7.20-7.80 (bs, 2H), 7.40 (m, 1H), 7.60 (m, 1H), 7.75 (s, 1H).

### Preparation 26

### Ethyl 5-amino-1-ethyl-6-oxo-3-thiophen-3-yl-1,6-dihydropyridazin-4-carboxylate

To a solution of the title product of Preparation 25 (3.8 g, 15.9 mmol) in dimethylformamide (45 ml), potassium carbonate (8.8 g, 63.6 mmol) was added. This mixture was heated to 70° C. for 1 h. Then it was cooled again to room temperature and bromoethane (4.8 ml, 63.9 mmol) in DMF (9 ml) was added dropwise. After heating at 70° C. for 18 h, the reaction mixture was poured into water and extracted repeatedly with Et₂O. This organic phase was washed with 4% NaHCO3, water and brine, dried over magnesium sulfate, filtered and evaporated to dryness. 3.8 g of the desired final compound were obtained as a solid (81% yield).

δ(CDCl₃): 0.95 (t, 3H), 1.40 (t, 3H), 4.10 (q, 2H), 4.25 (q, 2H), 7.05 (m, 4H), 7.30 (m, 1H).

### Preparation 27

### 1-Ethyl-6-oxo-3-phenyl-5-(quinolin-5-ylamino)-1,6-dihydropyridazine-4-carboxylic acid

A mixture of the title compound of Preparation 5 (1.0 g, 3.8 mmol), quinoline-5-boronic acid (1.33 g, 7.7 mmol), anhydrous cupric acetate (1.05 g, 7.7 mmol), triethylamine (2.12 ml, 15.4 mmol) and activated molecular sieves (2 g, 4 Å) in dry dichloromethane (40 ml) was stirred under air exposure at room temperature for 24 h. Acetic acid (0.88 ml, 15.4 mmol) was then added and the reaction was filtered. Finally, solvent was removed under reduced pressure. The resulting residue was purified by flash column cromathography (SiO₂, dichloromethane-ethyl acetate-methanol) to yield the title product (586 mg, 35% yield).

LRMS: m/Z 387 (M+1)⁺.

Retention Time: 9 min.

δ(DMSO-d₆): 1.36 (t, 3H), 4.20 (q, 2H), 7.33 (m, 6H), 7.63 (m, 2H), 7.88 (m, 1H), 8.41 (m, 1H), 8.90 (m, 1H), 9.13 (m, 1H), 12.46 (s, 1H).

### Preparation 28

### 1-Ethyl-6-oxo-3-phenyl-5-(pyridin-3-ylamino)-1,6-dihydropyridazine-4-carboxylic acid

To a stirred suspension of the title product of Example 15 (1.1 g, 3.02 mol) in ethanol (50 ml), 2N NaOH (2.3 ml, 4.6 mmol) was added dropwise and the resulting yellow solution was stirred at 60° C. for 4 h. Then it was let to cool down and solvent was removed under reduced pressure. The solid thus obtained was suspended in water and acified to pH 2 with HCl 2N. The solid thus obtained was filtered, washed with ethyl ether and dried to yield the title product (62%).

m.p. 255.1-256.7° C.

δ(DMSO-d₆): 1.33 (t, 3H), 4.17 (q, 2H), 7.26 (m, 1H), 7.38 (s, 5H), 7.46 (m, 1H), 8.29 (m, 2H), 9.02 (s, 1H), 13.00 (s, 1H).

### Preparation 29

### 1-Ethyl-5-[(4-methylpyridin-3-yl)amino]-6-oxo-3-phenyl-1,6-dihydropyridazine-4-carboxylic acid

Obtained as a solid (50%) from the title compound of Example 25 following the experimental procedure of Preparation 28.

LRMS: m/Z 351 (M+1)⁺.

Retention Time: 8 min.

δ(DMSO-d₆): 1.34 (t, 3H), 2.20 (s, 3H), 4.17 (q, 2H), 7.21 (m, 1H), 7.36 (m, 5H), 8.18 (s, 1H), 8.26 (d, 1H), 8.72 (s, 1H).

### Preparation 30

### 1-Ethyl-5-(isoquinolin-4-ylamino)-6-oxo-3-phenyl-1,6-dihydropyridazine-4-carboxylic acid

Obtained as a solid (86%) from the title compound of Example 30 following the experimental procedure of Preparation 28.

m.p. 269.5-270.4° C.

δ(DMSO-d₆): 1.37 (t, 3H), 4.20 (q, 2H), 7.35 (m, 5H), 7.68 (t, 1H), 7.78 (t, 1H), 7.97 (d, 1H), 8.12 (d, 1H), 8.27 (s, 1H), 9.07 (s, 1H), 9.17 (s, 1H), 12.5 (s, 1H).

### Preparation 31

### 1-Ethyl-3-(3-methylphenyl)-6-oxo-5-(pyridin-3-ylamino)-1,6-dihydropyridazine-4-carboxylic acid

Obtained as a solid (57%) from the title compound of Example 34 following the experimental procedure of Preparation 28.

LRMS: m/Z 351 (M+1)⁺.

Retention Time: 6.0 min*. *Chromatographic method B.

δ(DMSO-d₆): 1.33 (t, 3H), 2.31 (s, 3H), 4.16 (q, 2H), 7.20 (m, 5H), 7.46 (m, 1H), 8.27 (d, 1H), 8.34 (s, 1H), 8.99 (s, 1H), 12.98 (bs, 1H).

### Preparation 32

### 1-Ethyl-3-(3-fluorophenyl)-6-oxo-5-(pyridin-3-ylamino)-1,6-dihydropyridazine-4-carboxylic acid

Obtained as a solid (80%) from the title compound of Example 36 following the experimental procedure of Preparation 28.

LRMS: m/Z 355 (M+1)⁺.

Retention Time: 8 min.

δ(DMSO-d₆): 1.33 (t, 3H), 4.18 (q, 2H), 7.28 (m, 3H), 7.47 (m, 1H), 7.66 (m, 1H), 7.91 (m, 1H), 8.42 (m, 1H), 8.52 (s, 1H), 9.42 (s, 1H).

### Preparation 33

### 1-Ethyl-3-(4-fluorophenyl)-6-oxo-5-(pyridin-3-ylamino)-1,6-dihydropyridazine-4-carboxylic acid

Obtained as a solid (90%) from the title compound of Example 38 following the experimental procedure of Preparation 28.

LRMS: m/Z 355 (M+1)⁺.

Retention Time: 8 min.

δ(DMSO-d₆): 1.30 (t, 3H), 4.16 (q, 2H), 7.22 (m, 3H), 7.42 (m, 3H), 8.27 (m, 1H), 8.35 (s, 1H), 9.07 (s, 1H), 13.07 (bs, 1H).

### Preparation 34

### 1-Ethyl-6-oxo-5-(pyridin-3-ylamino)-3-(2-thienyl)-1,6-dihydropyridazine-4-carboxylic acid

To a suspension of the title product of Example 43 (1.6 g, 4.3 mmol) in methanol (16 ml) 2N sodium hydroxide (4.3 ml, 8.7 mmol) was dropwise added. The reaction mixture was heated overnight at 80° C. Then it was acidified at room temperature with 2N HCl until pH=5, precipitating a white solid. After cooling in an ice-bath, 0.77 g of the desired final compound was isolated by filtration. (52% yield).

δ(CDCl₃): 1.33 (t, 3H), 4.15 (q, 2H), 7.04 (m, 1H), 7.15 (m, 1H), 7.30 (m, 1H), 7.51 (m, 1H), 7.59 (m, 1H), 8.32 (d, 1H), 8.36 (m, 1H), 8.96 (s, 1H).

### Preparation 35

### 1-Ethyl-5-(4-methylpyridin-3-ylamino)-6-oxo-3-thiophen-2-yl-1,6-dihydropyridazin-4-carboxylic acid

Obtained as a solid (93%) from the title product of Example 49 following the experimental procedure described in Preparation 34.

δ(DMSO-d₆): 1.35 (t, 3H), 2.20 (s, 3H), 4.15 (q, 2H), 7.05 (m, 1H), 7.10 (m, 1H), 7.25 (m, 1H), 7.60 (m, 1H), 8.20 (s, 1H), 8.30 (m, 1H), 8.70 (s, 1H).

### Preparation 36

### 1-Ethyl-5-(isoquinolin-4-ylamino)-6-oxo-3-(2-thienyl)-1,6-dihydropyridazine-4-carboxylic acid

Obtained as a solid (45%) from the title product of Example 52 following the experimental procedure described in Preparation 34.

δ(DMSO-d₆): 1.40 (t, 3H), 4.20 (q, 2H), 7.00 (m, 1H), 7.05 (m, 1H), 7.60 (m, 1H), 7.80 (m, 1H), 7.90 (m, 1H), 8.05 (m, 1H), 8.25 (m, 1H), 8.45 (bs, 1H), 9.20 (s, 1H), 9.40 (bs, 1H).

### Preparation 37

### 1-Ethyl-5-(isoquinolin-4-ylamino)-6-oxo-3-(3-thienyl)-1,6-dihydropyridazine-4-carboxylic acid

Obtained as a solid (57%) from the title product of Example 58 following the experimental procedure described in Preparation 34.

δ(DMSO-d₆): 1.40 (t, 3H), 4.20 (q, 2H), 7.10 (m, 1H), 7.50 (m, 2H), 7.70 (m, 1H), 7.80 (m, 1H), 7.95 (m, 1H), 8.15 (m, 1H), 8.30 (bs, 1H), 9.05 (s, 1H), 9.20 (bs, 1H).

### Preparation 38

### Ethyl 3-(4-methylphenyl)-3-oxopropanoate

To an ice-cooled solution of sodium hydride (3.13 g, 78.25 mmol) in diethyl carbonate (75 ml) was dropwise added a solution of 4-methyl acetophenone (5 g, 37.3 mmole) in diethyl carbonate (3 ml). The mixture was stirred at room temperature for 30 min and at 85° C. for 2 hours, then poured into ice-water-acetic acid (50:50:1 vol.), extracted with ethyl acetate, washed with brine, dried and concentrated to yield an oil which was distilled (120° C., 0.1 mbar) to afford a colourless oil (6.98 g, 91% yield).

δ(CDCl3): 1.26 (t, 3H), 2.42 (s, 3H), 3.97 (s, 2H), 4.21 (q, 2H), 7.28 (d, 2H), 7.84 (d, 2H).

### Preparation 39

### Ethyl 4-(4-methylbenzoyl)-5-oxo-2,5-dihydroisoxazole-3-carboxylate

To an ice-cooled suspension of sodium hydride (3.46 g, 144.1 mmol) in tetrahydrofuran (200 ml) the title compound of Preparation 38 (14.1 g, 68.6 mmol) in 70 ml of tetrahydrofuran was dropwise added, and the mixture stirred at 0° C. for 20 min. A solution of ethyl chloro(hydroximino)acetate (11.4 g, 75.5 mmol) in tetrahydrofuran (70 ml) was slowly added and the final mixture was stirred at 0° C. for 30 min. and at room temperature for one additional hour. The reaction was quenched by the addition of water (1.23 ml, 68.3 mmole), the mixture was concentrated and the residue thus obtained was suspended in water (200 ml), acidified with HCl 2N to pH=1 and extracted with ethyl acetate (150 ml×4). The combined organic layers were washed with brine, dried and concentrated under reduced pressure to yield the title product as a yellowish oil (19.6 g, 95% yield).

δ(DMSO-d6): 1.18 (t, 3H), 2.35 (s, 3H), 4.10 (q, 2H), 7.18 (d, 2H), 7.60 (d, 2H).

LRMS (m/z): 276 (M+1)⁺.

Retention Time: 6.62* min. *Chromatographic method B.

### Preparation 40

### 4-(4-Methylphenyl)-1,6-dihydroisoxazolo[3,4-d]pyridazine-3,7-dione

Hydrazine monohydrate (12.17 g, 243 mmol) was added dropwise to a solution of the title compound of Preparation 39 (19.6 g, 68.5 mmol) in dry ethanol (171 ml) and the resulting mixture was stirred overnight. After cooling with an ice bath, a precipitate was formed which was collected by filtration and washed with cold ethanol to yield the title compound (18.6 g, 95% yield) as a light brown solid.

δ(DMSO-d6): 2.35 (s, 3H), 7.18 (d, 2H), 7.80 (d, 2H).

LRMS (m/z): 244 (M+1)⁺.

Retention Time: 5.82* min. *Chromatographic method B

### Preparation 41

### 5-Amino-3-(4-methylphenyl)-6-oxo-1,6-dihydropyridazine-4-carboxylic acid

Hydrazine monohydrate (13.1 g, 263 mmol) was added dropwise to a suspension of the title compound of Preparation 40 (16.8 g, 68.5 mmol) in dry ethanol (210 ml) and the resulting mixture was refluxed overnight. After cooling to room temperature, the mixture was further cooled with an ice bath and a precipitate was formed which was collected by filtration and washed with cold ethanol and diethyl ether to yield the title compound (10.1 g, 60% yield) as a yellow solid.

δ(DMSO-d6): 2.30 (s, 3H), 6.60 (bs, 2H), 7.10 (d, 2H), 7.30 (d, 2H).

LRMS (m/z): 246 (M+1)⁺.

Retention Time: 6.02 min.

### Preparation 42

### Ethyl 5-amino-1-ethyl-3-(4-methylphenyl)-6-oxo-1,6-dihydropyridazine-4-carboxylate

To a suspension of the title compound of Preparation 41 (8.5 g, 34.7 mmol) and anhydrous potassium carbonate (28.7 g, 208 mmol) in dry dimethylformamide (116 ml) was added ethyl bromide (22.69 g, 208 mmol) and the resulting mixture was stirred at 60° C. overnight. The mixture was cooled down, filtered, concentrated and the residue thus obtained was diluted with dichloromethane (350 ml), washed with water and brine, dried and concentrated to yield 13.4 g of a solid which was recrystalised from EtOH to afford the title compound (6.96 g, 67% yield) as yellow crystals.

δ(DMSO-d6): 0.8 (t, 3H), 1.28 (t, 3H), 2.38 (s, 3H), 3.98 (q, 2H), 4.10 (q, 2H), 7.20 (s, 4H), 7.38 (bs, 2H).

LRMS (m/z): 302 (M+1)⁺.

Retention Time: 9.67 min.

### Preparation 43

### 1-Ethyl-3-(4-methylphenyl)-6-oxo-5-(pyridin-3-ylamino)-1,6-dihydropyridazine-4-carboxylic acid

To a stirred suspension of the title product of Example 65 (350 mg, 0.92 mol) in ethanol (3 ml) 2N NaOH (0.78 ml, 1.57 mol) was added dropwise and the resulting mixture was stirred at 60° C. for 3 hours. Then it was let to cool down and solvent was removed under reduced pressure. The residue was redissolved in water (20 ml) and the solution was adjusted to pH=2 with HCl 2N. The solid thus obtained was filtered, washed with ethyl ether and dried to yield the title product (48%).

δ(DMSO-d6): 1.32 (t, 3H), 2.32 (s, 3H), 4.16 (q, 2H), 7.18 (d, 2H), 7.26 (m, 1H), 7.28 (d, 2H), 7.45 (d, 1H), 8.28 (d, 1H), 8.33 (s, 1H), 8.98 (s, 1H).

LRMS (m/z): 351 (M+1)⁺.

Retention Time: 9 min.

### Preparation 44

### 1-Ethyl-5-(isoquinolin-4-ylamino)-3-(4-methylphenyl)-6-oxo-1,6-dihydropyridazine-4-carboxylic acid

Obtained as a solid (62%) from the title compound of Example 66 following the procedure of Preparation 43.

δ(DMSO-d6): 1.37 (t, 3H), 2.28 (s, 3H), 4.20 (q, 2H), 7.13 (d, 2H), 7.24 (d, 2H), 7.69 (t, 1H), 7.78 (t, 1H), 7.97 (d, 1H), 8.13 (d, 1H), 8.27 (s, 1H), 9.04 (s, 1H), 9.18 (s, 1H).

LRMS (m/z): 401 (M+1)⁺.

Retention Time: 11 min.

### Preparation 45

### 1-Ethyl-3-(4-methylphenyl)-5-[(4-methylpyridin-3-yl)amino]-6-oxo-1,6-dihydropyridazine-4-carboxylic acid

Obtained as a solid (85%) from the title compound of Example 67 following the procedure of Preparation 43.

δ(DMSO-d6): 1.34 (t, 3H), 2.20 (s, 3H), 2.30 (s, 3H), 4.17 (q, 2H), 7.15 (d, 2H), 7.23 (d, 1H), 7.26 (d, 2H), 8.19 (s, 1H), 8.28 (d, 1H), 8.67 (s, 1H).

LRMS (m/z): 365 (M+1)⁺.

Retention Time: 9 min.

### Preparation 46

### 1-Chloroethyl isopropyl carbonate

To a solution of isopropanol (1.09 g, 18.27 mmol) and pyridine (1.45 g, 18.35 mmol) in of dichloromethane (30 ml) at −78° C. was dropwise added (10 minutes) 1-chloroethyl chloroformate (2.66 g, 18.60 mmol) under argon. After the addition, the cooling bath was removed and the mixture was allowed to warm to rt and stirred at that temperature overnight. The reaction was diluted with additional dichloromethane (20 ml), washed with brine and dried over anhydrous sodium sulfate. Removal of the solvent under reduced pressure afforded the title compound as a colourless oil (3 g, 97% yield).

δ(CDCl3): 1.33 (d, 3H), 1.35 (d, 3H), 1.84 (d, 3H), 4.95 (m, 1H), 6.43 (q, 1H).

### Preparation 47

### 1-Chloroethyl cyclohexyl carbonate

Obtained as an oil (96%) from cyclohexanol and 1-chloroethyl chloroformate following the procedure of Preparation 46.

δ (CDCl3): 1.23-2.0 (m, 10H), 1.84 (d, 3H), 4.69 (m, 1H), 6.43 (q, 1H).

### Preparation 48

### 1-Chloroethyl ethyl carbonate

Obtained as an oil (90%) from ethanol and 1-chloroethyl chloroformate following the procedure of Preparation 46.

δ(CDCl3): 1.27 (t, 3H), 1.82 (d, 3H), 4.22 (q, 2H), 6.42 (q, 1H).

### Preparation 49

### 1-Ethyl-6-oxo-3-phenyl-5-(thieno[2,3-b]pyridin-3-ylamino)-1,6-dihydropyridazine-4-carboxylic acid

Obtained as a solid (58%) from the title compound of Example 78 following the experimental procedure of Preparation 28.

LRMS: m/Z 393 (M+1)⁺.

### Preparation 50

### 1-Ethyl-5-(pyridin-3-ylamino)-6-oxo-3-thiophen-3-yl-1,6-dihydropyridazin-4-carboxylic

Obtained as a solid (90%) from the title product of Example 108 following the experimental procedure described in Preparation 34.

LRMS: m/Z 343 (M+1)⁺.

Retention Time: 7 min.

### Preparation 51

### 1-Ethyl-5-(4-methylpyridin-3-ylamino)-6-oxo-3-thiophen-3-yl-1,6-dihydropyridazin-4-carboxylic acid

Obtained as a solid (93%) from the title product of Example 119 following the experimental procedure described in Preparation 34.

LRMS: m/Z 357 (M+1)⁺.

Retention Time: 7 min.

### Preparation 52

### 1-Ethyl-5-([1,7]naphthyridin-5-ylamino)-6-oxo-3-phenyl-1,6-dihydro-pyridazine-4-carboxylic acid

Obtained as a solid (45%) from the title product of Example 177 following the experimental procedure described in Preparation 34.

LRMS: m/Z 388 (M+1)⁺.

Retention Time: 7.1 min.

### EXAMPLES

In the following tables some acronyms have been used with the following meanings:

| Acronym | Meaning |
|---|---|
| AcO | Acetyloxy |
| Et | Ethyl |
| Bn | Benzyl |
| BoC | tert-butyloxycarbonyl |
| Me | Methyl |
| Ph | Phenyl |

| EXAMPLE | R1 | R2 | R3 | R4 |
|---|---|---|---|---|
| 1 | Et | 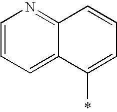 | 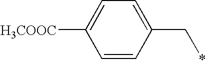 | Ph |
| 2 | Et | 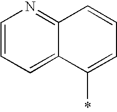 | Bn | Ph |
| 3 | Et | 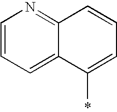 | 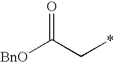 | Ph |
| 4 | Et | 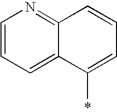 | 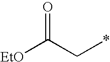 | Ph |
| 5 | Et | 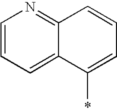 | 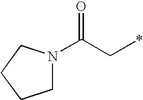 | Ph |
| 6 | Et | 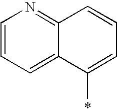 | 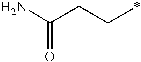 | Ph |
| 7 | Et | 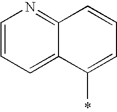 | 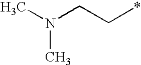 | Ph |
| 8 | Et | 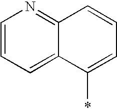 | 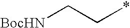 | Ph |
| 9 | Et | 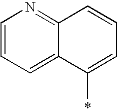 | 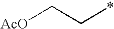 | Ph |
| 10 | Et | 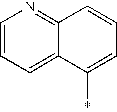 | 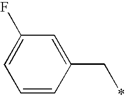 | Ph |
| 11 | Et | 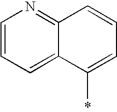 | 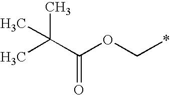 | Ph |
| 12 | Et | 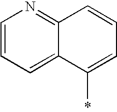 | 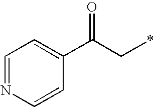 | Ph |
| 13 | Et | 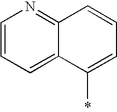 | 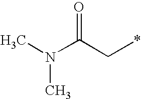 | Ph |
| 14 | Et | 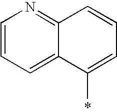 | 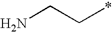 | Ph |
| 15 | Et | 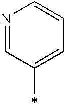 | Et | Ph |
| 16 | Et | 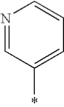 | 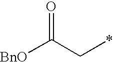 | Ph |
| 17 | Et | 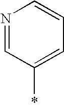 | 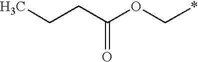 | Ph |
| 18 | Et | 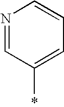 | 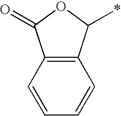 | Ph |
| 19 | Et | 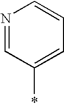 | 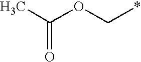 | Ph |
| 20 | Et | 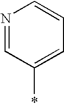 | 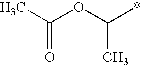 | Ph |
| 21 | Et | 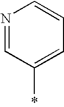 | 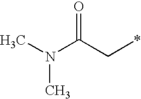 | Ph |
| 22 | Et | 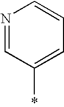 | Bn | Ph |
| 23 | Et | 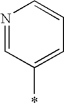 | 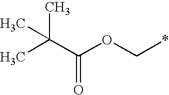 | Ph |
| 24 | Et | 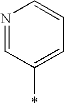 | 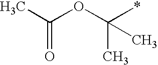 | Ph |
| 25 | Et | 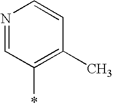 | Et | Ph |
| 26 | Et | 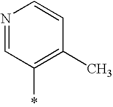 | 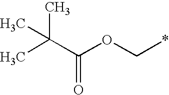 | Ph |
| 27 | Et | 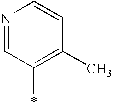 | 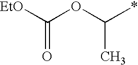 | Ph |
| 28 | Et | 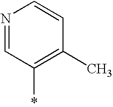 | 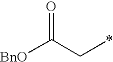 | Ph |
| 29 | Et | 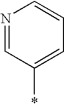 | 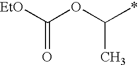 | Ph |
| 30 | Et | 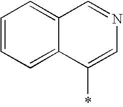 | Et | Ph |
| 31 | Et | 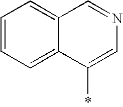 | 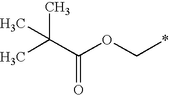 | Ph |
| 32 | Et | 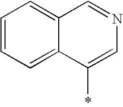 | 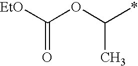 | Ph |
| 33 | Et | 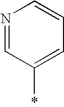 | 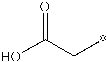 | Ph |
| 34 | Et | 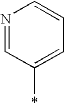 | Et | 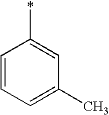 |
| 35 | Et | 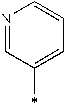 | 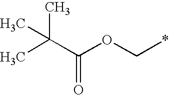 | 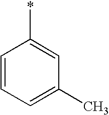 |
| 36 | Et | 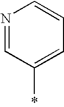 | Et | 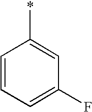 |
| 37 | Et | 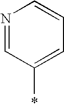 | 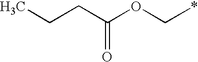 | 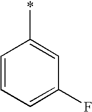 |
| 38 | Et | 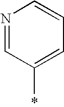 | Et | 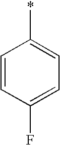 |
| 39 | Et | 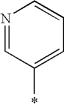 | 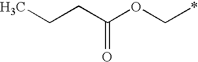 | 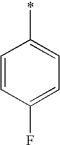 |
| 40 | Et | 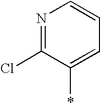 | Et | Ph |
| 41 | Et | 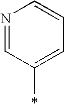 | Me | Ph |
| 42 | Et | 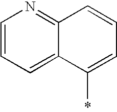 | Me | Ph |
| 43 | Et | 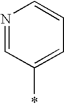 | Et | 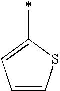 |
| 44 | Et | 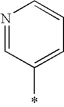 | 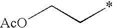 | 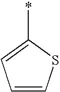 |
| 45 | Et | 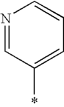 | 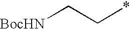 | 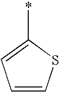 |
| 46 | Et | 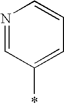 | 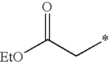 | 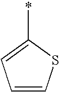 |
| 47 | Et | 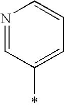 | 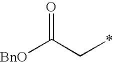 | 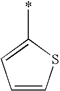 |
| 48 | Et | 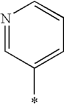 | Bn | 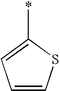 |
| 49 | Et | 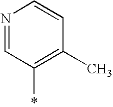 | Et | 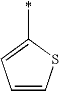 |
| 50 | Et | 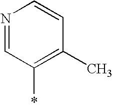 | 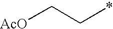 | 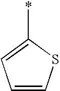 |
| 51 | Et | 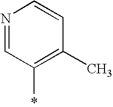 | 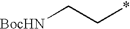 | 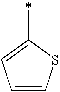 |
| 52 | Et | 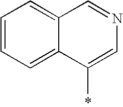 | Et | 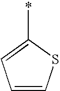 |
| 53 | Et | 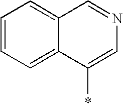 | 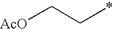 | 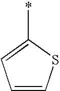 |
| 54 | Et | 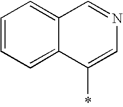 | 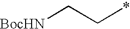 | 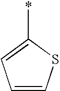 |
| 55 | Et | 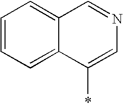 | 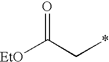 | 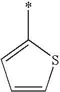 |
| 56 | Et | 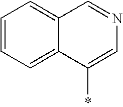 | 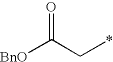 | 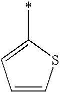 |
| 57 | Et | 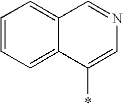 | Bn | 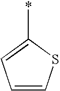 |
| 58 | Et | 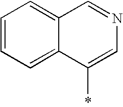 | Et | 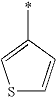 |
| 59 | Et | 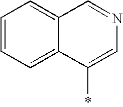 | 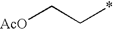 | 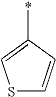 |
| 60 | Et | 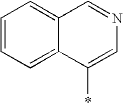 | 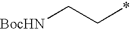 | 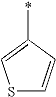 |
| 61 | Et | 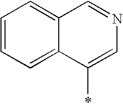 | 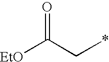 | 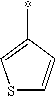 |
| 62 | Et | 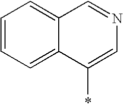 | 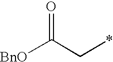 | 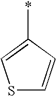 |
| 63 | Et | 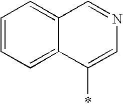 | 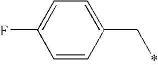 | 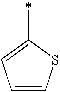 |
| 64 | Et | 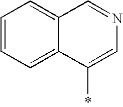 | 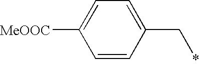 | 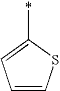 |
| 65 | Et | 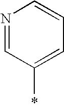 | Et | 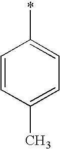 |
| 66 | Et | 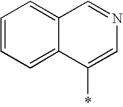 | Et | 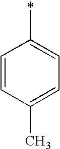 |
| 67 | Et | 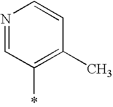 | Et | 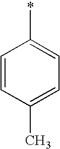 |
| 68 | Et | 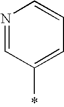 | 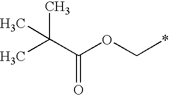 | 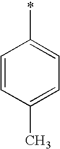 |
| 69 | Et | 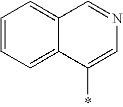 | 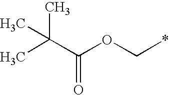 | 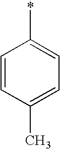 |
| 70 | Et | 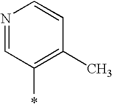 | 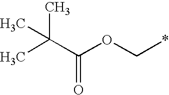 | 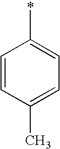 |
| 71 | Et | 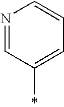 | 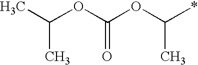 | Ph |
| 72 | Et | 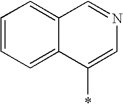 | 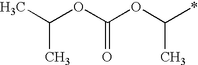 | Ph |
| 73 | Et | 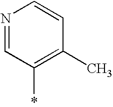 | 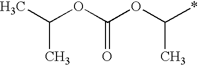 | Ph |
| 74 | Et | 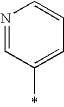 | 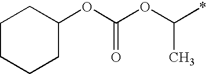 | Ph |
| 75 | Et | 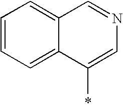 | 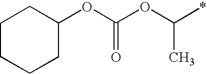 | Ph |
| 76 | Et | 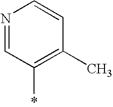 | 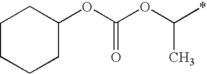 | Ph |
| 77 | Et | 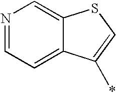 | Et | Ph |
| 78 | Et | 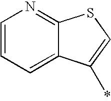 | Et | Ph |
| 79 | Et | 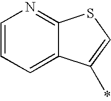 | 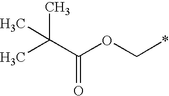 | Ph |
| 80 | Et | 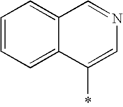 | 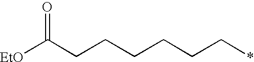 | 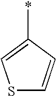 |
| 81 | Et | 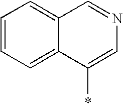 | 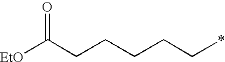 | 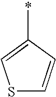 |
| 82 | Et | 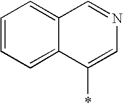 | 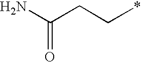 | 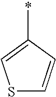 |
| 83 | Et | 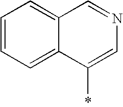 | Bn | 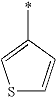 |
| 84 | Et | 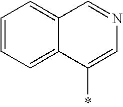 | 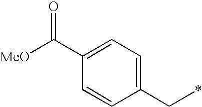 | 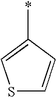 |
| 85 | Et | 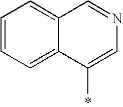 | 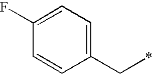 | 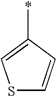 |
| 86 | Et | 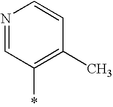 | 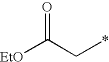 | 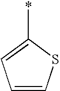 |
| 87 | Et | 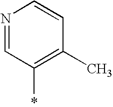 | 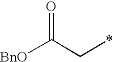 | 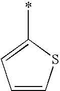 |
| 88 | Et | 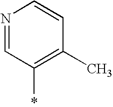 | Bn | 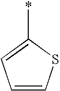 |
| 89 | Et | 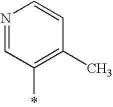 | 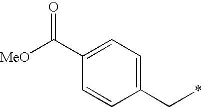 | 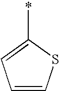 |
| 90 | Et | 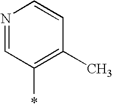 | 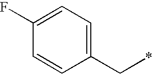 | 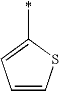 |
| 91 | Et | 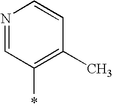 | 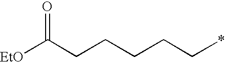 | 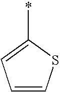 |
| 92 | Et | 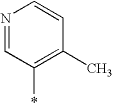 | 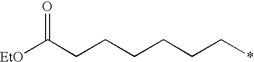 | 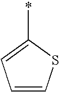 |
| 93 | Et | 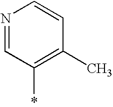 | 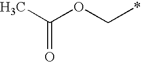 | 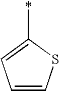 |
| 94 | Et | 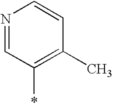 | 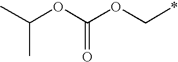 | 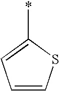 |
| 95 | Et | 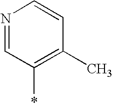 | 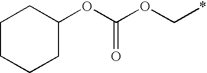 | 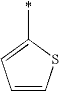 |
| 96 | Et | 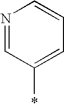 | 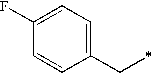 | 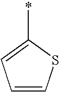 |
| 97 | Et | 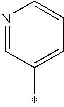 | 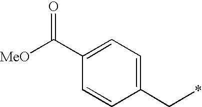 | 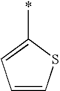 |
| 98 | Et | 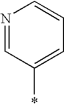 | 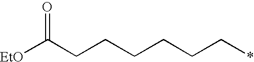 | 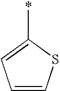 |
| 99 | Et | 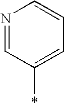 | 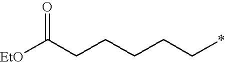 | 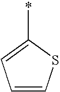 |
| 100 | Et | 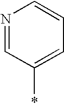 | 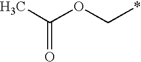 | 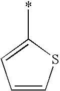 |
| 101 | Et | 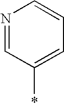 | 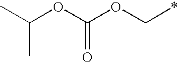 | 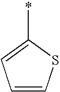 |
| 102 | Et | 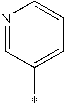 | 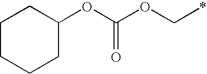 | 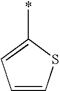 |
| 103 | Et | 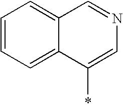 | 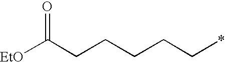 | 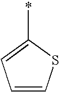 |
| 104 | Et | 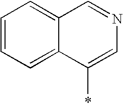 | 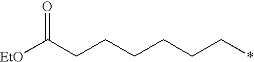 | 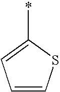 |
| 105 | Et | 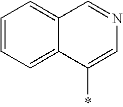 | 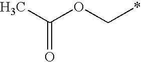 | 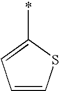 |
| 106 | Et | 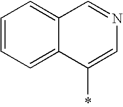 | 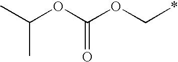 | 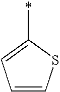 |
| 107 | Et | 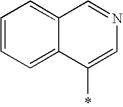 | 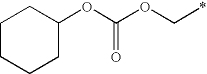 | 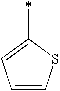 |
| 108 | Et | 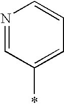 | Et | 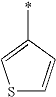 |
| 109 | Et | 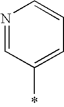 | 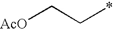 | 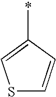 |
| 110 | Et | 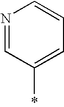 | 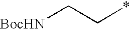 | 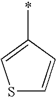 |
| 111 | Et | 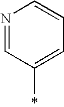 | 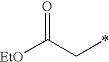 | 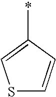 |
| 112 | Et | 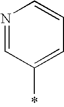 | 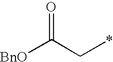 | 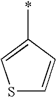 |
| 113 | Et | 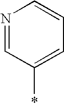 | Bn | 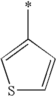 |
| 114 | Et | 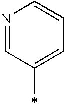 | 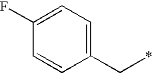 | 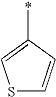 |
| 115 | Et | 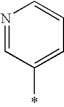 | 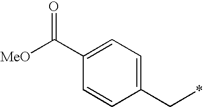 | 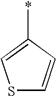 |
| 116 | Et | 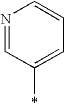 | 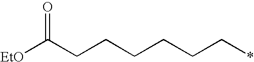 | 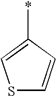 |
| 117 | Et | 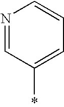 | 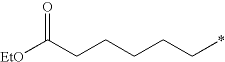 | 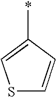 |
| 118 | Et | 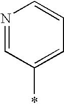 | 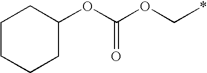 | 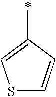 |
| 119 | Et | 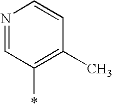 | Et | 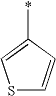 |
| 120 | Et | 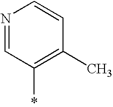 | 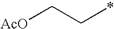 | 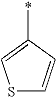 |
| 121 | Et | 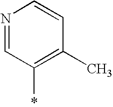 | 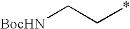 | 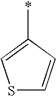 |
| 122 | Et | 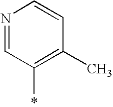 | 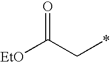 | 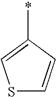 |
| 123 | Et | 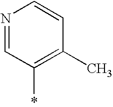 | 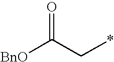 | 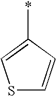 |
| 124 | Et | 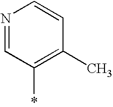 | Bn | 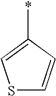 |
| 125 | Et | 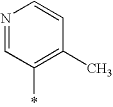 | 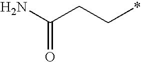 | 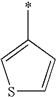 |
| 126 | Et | 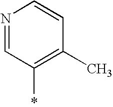 | 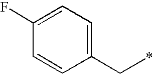 | 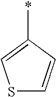 |
| 127 | Et | 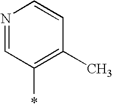 | 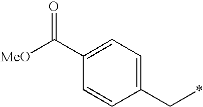 | 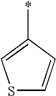 |
| 128 | Et | 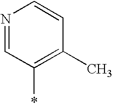 | 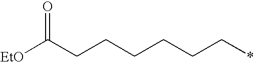 | 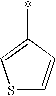 |
| 129 | Et | 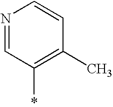 | 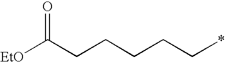 | 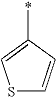 |
| 130 | Et | 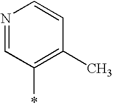 | 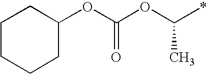 | Ph |
| 131 | Et | 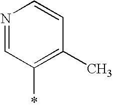 | 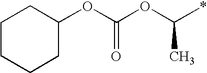 | Ph |
| 132 | Et | 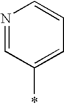 | 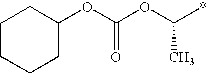 | Ph |
| 133 | Et | 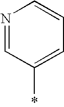 | 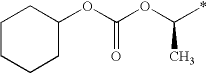 | Ph |
| 134 | Et | 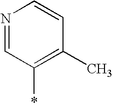 | 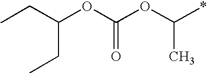 | Ph |
| 135 | Et | 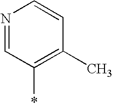 | 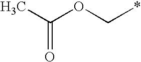 | Ph |
| 136 | Et | 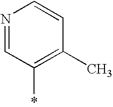 | 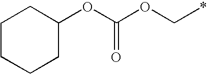 | Ph |
| 137 | Et | 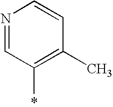 | 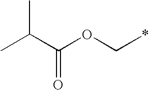 | Ph |
| 138 | Et | 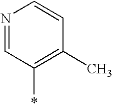 | 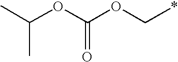 | Ph |
| 139 | Et | 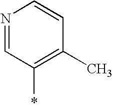 | 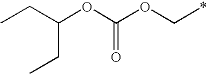 | Ph |
| 140 | Et | 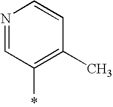 | 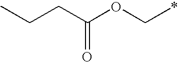 | Ph |
| 141 | Et | 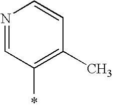 | 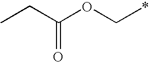 | Ph |
| 142 | Et | 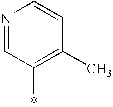 | 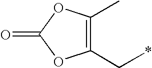 | Ph |
| 143 | Et | 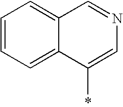 | 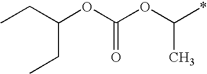 | Ph |
| 144 | Et | 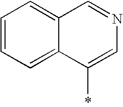 | 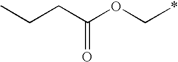 | Ph |
| 145 | Et | 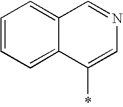 | 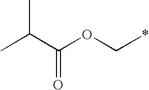 | Ph |
| 146 | Et | 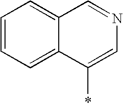 | 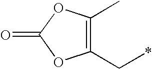 | Ph |
| 147 | Et | 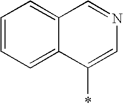 | 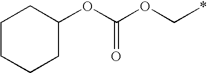 | Ph |
| 148 | Et | 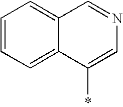 | 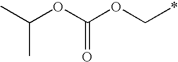 | Ph |
| 149 | Et | 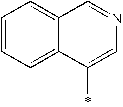 | 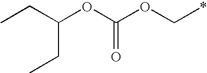 | Ph |
| 150 | Et | 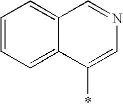 | 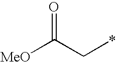 | Ph |
| 151 | Et | 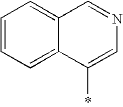 | 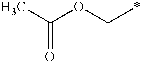 | Ph |
| 152 | Et | 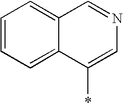 | 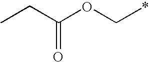 | Ph |
| 153 | Et | 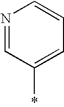 | 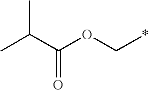 | Ph |
| 154 | Et | 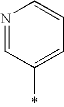 | 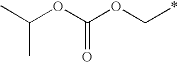 | Ph |
| 155 | Et | 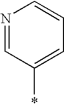 | 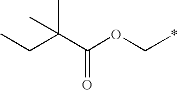 | Ph |
| 156 | Et | 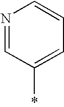 | 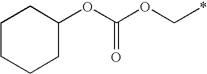 | Ph |
| 157 | Et | 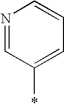 | 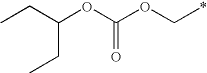 | Ph |
| 158 | Et | 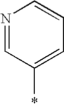 | 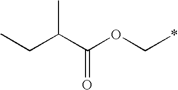 | Ph |
| 159 | Et | 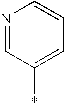 | 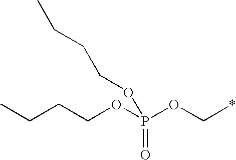 | Ph |
| 160 | Et | 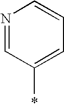 | 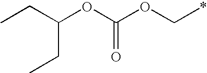 | 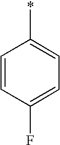 |
| 161 | Et | 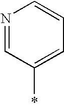 | 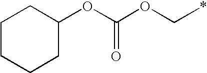 | 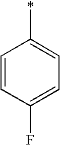 |
| 162 | Et | 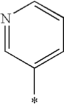 | 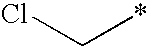 | Ph |
| 163 | Et | 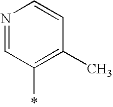 | 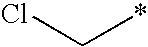 | Ph |
| 164 | Et | 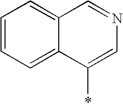 | 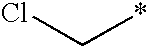 | Ph |
| 165 | Et | 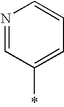 | 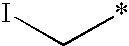 | Ph |
| 166 | Et | 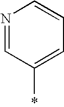 | 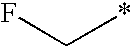 | Ph |
| 167 | Et | 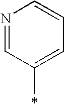 | 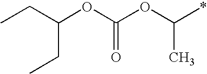 | Ph |
| 168 | Et | 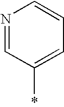 | 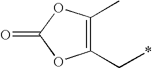 | Ph |
| 169 | Et | 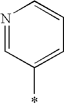 | 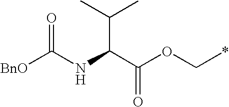 | Ph |
| 170 | Et | 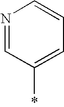 | 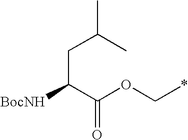 | Ph |
| 171 | Et | 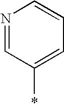 | 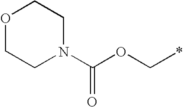 | Ph |
| 172 | Et | 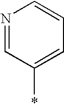 | 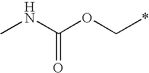 | Ph |
| 173 | Et | 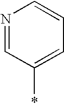 | 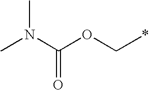 | Ph |
| 174 | Et | 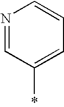 | 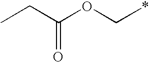 | Ph |
| 175 | Et | 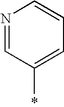 | 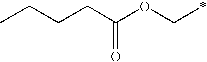 | Ph |
| 176 | Et | 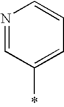 | 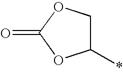 | Ph |
| 177 | Et | 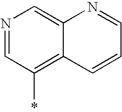 | Et | 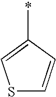 |
| 178 | Et | 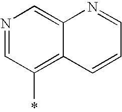 | 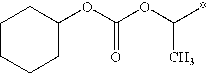 | Ph |

### Example 1

### 4-(Methoxycarbonyl)benzyl 1-ethyl-6-oxo-3-phenyl-5-(quinolin-5-ylamino)-1,6-dihydropyridazine-4-carboxylate

To a stirred mixture of the title product of Preparation 27 (90 mg, 0.23 mmol) in dry acetone (2 ml), potassium carbonate (36 mg, 0.26 mmol) was added portionwise and the resulting mixture was stirred for a while. Then, methyl 4-bromomethylbenzoate (47 mg, 0.2 mmol) was added dropwise and the final mixture was stirred at 40° C. for 20 h. Solvent was then removed under reduced pressure and the resulting residue was purified by flash column cromathography (SiO₂, hexane-ethyl acetate) to yield the title product (60 mg, 50% yield).

LRMS: m/Z 535 (M+1)⁺.

Retention Time: 17 min.

δ(CDCl₃): 1.49 (t, 3H), 3.90 (m, 4H), 4.36 (q, 2H), 6.55 (m, 2H), 7.30 (m, 6H), 7.37 (m, 1H), 7.62 (m, 1H), 7.78 (m, 2H), 8.05 (m, 2H), 8.43 (m, 1H), 8.94 (m, 1H).

### Example 2

### Benzyl 1-ethyl-6-oxo-3-phenyl-5-(quinolin-5-ylamino)-1,6-dihydropyridazine-4-carboxylate

Obtained as a solid (57%) from the title product of Preparation 27 and benzyl bromide following the experimental procedure of Example 1.

LRMS: m/Z 477 (M+1)⁺.

Retention Time: 17 min.

δ(CDCl₃): 1.48 (t, 3H), 3.86 (s, 2H), 4.36 (q, 2H), 6.52 (m, 2H), 7.15 (m, 2H), 7.31 (m, 7H), 7.40 (m, 1H), 7.60 (m, 1H), 8.06 (m, 2H), 8.45 (m, 1H), 8.98 (m, 1H).

### Example 3

### 2-(Benzyloxy)-2-oxoethyl 1-ethyl-6-oxo-3-phenyl-5-(quinolin-5-ylamino)-1,6-dihydropyridazine-4-carboxylate

Obtained as a solid (38%) from the title product of Preparation 27 and benzyl bromoacetate following the experimental procedure of Example 1. Dry DMF was used as solvent.

LRMS: m/Z 535 (M+1)⁺.

Retention Time: 17 min.

δ(CDCl₃): 1.50 (t, 3H), 3.27 (s, 2H), 4.36 (q, 2H), 4.95 (s, 2H), 7.26 (m, 10H), 7.43 (m, 2H), 7.54 (m, 1H), 7.99 (d, 1H), 8.17 (s, 1H), 8.49 (d, 1H), 8.95 (m, 1H).

### Example 4

### 2-Ethoxy-2-oxoethyl 1-ethyl-6-oxo-3-phenyl-5-(quinolin-5-ylamino)-1,6-dihydropyridazine-4-carboxylate

Obtained as a solid (56%) from the title product of Preparation 27 and ethyl bromoacetate following the experimental procedure of Example 1. Dry DMF was used as solvent.

LRMS: m/Z 473 (M+1)⁺.

Retention Time: 15 min.

δ(CDCl₃): 1.12 (t, 3H), 1.50 (t, 3H), 3.22 (s, 2H), 3.98 (q, 2H), 4.36 (q, 2H), 7.32 (m, 4H), 7.43 (m, 2H), 7.45 (m, 1H), 7.61 (m, 1H), 8.03 (d, 1H), 8.17 (s, 1H), 8.50 (d, 1H), 8.99 (s, 1H).

### Example 5

### 2-Oxo-2-pyrrolidin-1-ylethyl 1-ethyl-6-oxo-3-phenyl-5-(quinolin-5-ylamino)-1,6-dihydropyridazine-4-carboxylate

Obtained as a solid (66%) from the title product of Preparation 27 and 2-oxo-2-pyrrolidin-1-yl-ethyl chloroacetate following the experimental procedure of Example 1. Dry DMF was used as solvent.

LRMS: m/Z 498 (M+1)⁺.

Retention Time: 14 min.

δ(CDCl₃): 1.50 (t, 3H), 1.77 (m, 4H), 2.79 (t, 2H), 3.25 (t, 2H), 3.37 (s, 2H), 4.34 (q, 2H), 7.33 (m, 3H), 7.48 (m, 3H), 7.61 (m, 1H), 8.00 (d, 1H), 8.34 (s, 1H), 8.52 (d, 1H), 8.95 (m, 1H).

### Example 6

### 3-Amino-3-oxopropyl 1-ethyl-6-oxo-3-phenyl-5-(quinolin-5-ylamino)-1,6-dihydropyridazine-4-carboxylate

Obtained as a solid (29%) from the title product of Preparation 27 and 3-chloropropionamide hydrochloride following the experimental procedure of Example 1. Dry DMF was used as solvent.

LRMS: m/Z 458 (M+1)⁺.

Retention Time: 11 min.

δ(CDCl₃): 1.50 (m, 5H), 3.25 (t, 2H), 4.36 (q, 2H), 4.79 (m, 1H), 4.90 (m, 1H), 7.36 (m, 5H), 7.52 (m, 1H), 7.65 (m, 1H), 8.05 (m, 2H), 8.48 (d, 1H), 9.00 (m, 1H).

### Example 7

### 2-(Dimethylamino)ethyl 1-ethyl-6-oxo-3-phenyl-5-(quinolin-5-ylamino)-1,6-dihydropyridazine-4-carboxylate

To a stirred mixture of the title product of Preparation 27 (80 mg, 0.23 mmol) in dry acetone (2 ml), potassium carbonate (70 mg, 0.50 mmol) was added portionwise and the resulting mixture was stirred for a while. Then, (2-chloroethyl)dimethylamine hydrochloride (36 mg, 0.25 mmol) was added dropwise and the final mixture was stirred at 40° C. for 24 h. Then potassium iodide (42 mg, 0.25 mmol) was added and the final mixture was stirred at rt for 3 days. Solvent was then removed under reduced pressure and the resulting residue was partitioned between water and ethyl acetate and the organic layer was washed with 4% NaHCO3 and brine. Finally it was purified by flash column chromatography (SiO₂, dichloromethane-ethyl acetate-methanol) to yield the title product (30 mg, 29% yield).

LRMS: m/Z 458 (M+1)⁺.

Retention Time: 8 min.

### Example 8

### 2-[(tert-Butoxycarbonyl)amino]ethyl 1-ethyl-6-oxo-3-phenyl-5-(quinolin-5-ylamino)-1,6-dihydropyridazine-4-carboxylate

Obtained as a solid (29%) from the title product of Preparation 27 and (2-bromoethyl)-carbamic acid tert-butyl ester following the experimental procedure of Example 1.

LRMS: m/Z 530 (M+1)⁺.

Retention Time: 16 min.

δ(CDCl₃): 1.40 (s, 9H), 1.48 (t, 3H), 2.60 (m, 2H), 3.01 (m, 2H), 3.62 (m, 1H), 4.36 (q, 2H), 7.34 (m, 6H), 7.48 (m, 1H), 7.60 (m, 1H), 8.05 (m, 2H), 8.46 (d, 1H), 8.98 (s, 1H).

### Example 9

### 2-(Acetyloxy)ethyl 1-ethyl-6-oxo-3-phenyl-5-(quinolin-5-ylamino)-1,6-dihydropyridazine-4-carboxylate

Obtained as a solid (53%) from the title product of Preparation 27 acid 2-bromoethyl acetate following the experimental procedure of Example 1. Dry DMF was used as solvent.

LRMS: m/Z 473 (M+1)⁺.

Retention Time: 14 min.

δ(CDCl₃): 1.50 (t, 3H), 1.91 (s, 3H), 3.06 (m, 2H), 3.44 (m, 2H), 4.36 (q, 2H), 7.34 (m, 6H), 7.53 (m, 1H), 7.62 (m, 1H), 8.04 (m, 2H), 8.49 (d, 1H), 9.00 (s, 1H).

### Examples 10-13

### 3-Fluorobenzyl i-ethyl-6-oxo-3-phenyl-5-(quinolin-5-ylamino)-1,6-dihydropyridazine-4-carboxylate

### [(2,2-Dimethylpropanoyl)oxy]methyl 1-ethyl-6-oxo-3-phenyl-5-(quinolin-5-ylamino)-1,6-dihydropyridazine-4-carboxylate

### 2-Oxo-2-pyridin-4-ylethyl 1-ethyl-6-oxo-3-phenyl-5-(quinolin-5-ylamino)-1,6-dihydropyridazine-4-carboxylate

### 2-(Dimethylamino)-2-oxoethyl 1-ethyl-6-oxo-3-phenyl-5-(quinolin-5-ylamino)-1,6-dihydropyridazine-4-carboxylate

The title compounds were synthesized from the title compound of Preparation 27 and the corresponding bromide or chloride following the procedure of Example 1. The ESI/MS data and HPLC retention times are summarized in Table 1.

TABLE 1

| | ESI/MS | Retention |
|---|---|---|
| EXAMPLE | m/e | Time (min) |
| 10 | 495 | 17 |
| 11 | 501 | 17 |
| 12 | 506 | 14 |
| 13 | 472 | 12 |

### Example 14

### 2-Aminoethyl 1-ethyl-6-oxo-3-phenyl-5-(quinolin-5-ylamino)-1,6-dihydropyridazine-4-carboxylate

A solution of the title product of Example 8 (20 mg, 0.037 mmol) in ethanol saturated with HCl was stirred at rt for 1 h. Solvent was then removed under reduced pressure and to yield the title product (23 mg, 99% yield).

LRMS: m/Z 430 (M+1)⁺.

Retention Time: 8 min.

### Example 15

### Ethyl 1-ethyl-6-oxo-3-phenyl-5-(pyridin-3-ylamino)-1,6-dihydropyridazine-4-carboxylate

A mixture of the title compound of Preparation 4 (6.0 g, 20.9 mmol), 3-bromopyridine (2.41 ml, 25.1 mmol), anhydrous cuprous iodide (398 mg, 2.1 mmol), N,N′-dimethylethylenediamine (0.44 ml, 4.18 mmol) and potassium carbonate (6.1 g, 43.9 mmol) in dry dioxane (20 ml) was stirred under argon at 130° C. for 48 h. It was let to cool down and filtered. The precipitate was thoroughly washed with dichloromethane. Finally, solvent was removed under reduced pressure. The resulting residue was purified by flash column chromatography (SiO₂, dichloromethane-ethyl acetate) to yield the title product (1.22 g, 18% yield).

LRMS: m/Z 365 (M+1)⁺.

Retention Time: 14 min.

δ (CDCl₆): 0.75 (t, 3H), 1.45 (t, 3H), 3.43 (q, 2H), 4.31 (q, 2H), 7.24 (m, 1H), 7.37 (s, 5H), 7.47 (m, 1H), 7.93 (s, 1H), 8.44 (m, 1H), 8.47 (m, 1H).

### Example 16

### 2-(Benzyloxy)-2-oxoethyl 1-ethyl-6-oxo-3-phenyl-5-(pyridin-3-ylamino)-1,6-dihydropyridazine-4-carboxylate

To a stirred mixture of the title product of Preparation 28 (80 mg, 0.24 mmol) in dry DMF (2 ml), potassium carbonate (66 mg, 0.47 mmol) was added portionwise and the resulting mixture was stirred for a while. Then, benzyl bromoacetate (42 μl, 0.26 mmol) was added dropwise and the final mixture was stirred at rt for 3 h. It was poured onto water and extracted with ethyl ether three times. The combined organic layers were washed with brine and dried. Solvent was then removed under reduced pressure and the resulting residue was purified by flash column chromatography (SiO₂, dichloromethane-ethyl acetate) to yield the title product (58 mg, 50% yield).

LRMS: m/Z 485 (M+1)⁺.

Retention Time: 16 min.

δ(CDCl₃): 1.45 (t, 3H), 3.91 (s, 2H), 4.31 (q, 2H), 5.09 (s, 2H), 7.18 (m, 1H), 7.30 (m, 2H), 7.35 (m, 7H), 7.44 (m, 2H), 8.15 (s, 1H), 8.43 (d, 1H), 8.48 (m, 1H).

### Example 17

### (Butyryloxy)methyl 1-ethyl-6-oxo-3-phenyl-5-(pyridin-3-ylamino)-1,6-dihydropyridazine-4-carboxylate

To a stirred mixture of the title product of Preparation 28 (30 mg, 0.09 mmol) in dry DMF (1 ml), diisopropylethylamine (18 μl, 0.107 mmol) was added dropwise and the resulting mixture was stirred for a while. Then, chloromethyl butyrate (10 μl, 0.10 mmol) was added dropwise and the final mixture was stirred at 50° C. for 4 h and then at rt for 2 days. Solvent was removed under reduced pressure and the resulting residue was purified by flash column chromatography (SiO₂, hexane-ethyl acetate) to yield the title product (40 mg, 52% yield).

LRMS: m/Z 437 (M+1)⁺.

Retention Time: 15 min.

δ(DMSO-d₆): 0.86 (t, 3H), 1.35 (t, 3H), 1.46 (m, 2H), 2.14 (t, 2H), 4.16 (q, 2H), 4.86 (s, 2H), 7.26 (m, 2H), 7.36 (m, 4H), 7.50 (m, 1H), 8.36 (m, 2H), 9.35 (s, 1H).

### Example 18

### 3-Oxo-1,3-dihydro-2-benzofuran-1-yl 1-ethyl-6-oxo-3-phenyl-5-(pyridin-3-ylamino)-1,6-dihydropyridazine-4-carboxylate

Obtained as a solid (89%) from the title product of Preparation 28 and 3-bromophthalide following the experimental procedure of Example 16.

LRMS: m/Z 469 (M+1)⁺.

Retention Time: 15 min.

δ(DMSO-d₆): 1.32 (t, 3H), 4.16 (q, 2H), 6.55 (s, 1H), 7.04 (d, 1H), 7.36 (m, 6H), 7.56 (m, 1H), 7.67 (m, 1H), 7.73 (m, 2H), 7.80 (m, 1H), 8.36 (m, 1H), 8.46 (s, 1H), 9.46 (s, 1H).

### Example 19

### (Acetyloxy)methyl 1-ethyl-6-oxo-3-phenyl-5-(pyridin-3-ylamino)-1,6-dihydropyridazine-4-carboxylate

Obtained as a solid (74%) from the title product of Preparation 28 and bromomethyl acetate following the experimental procedure of Example 17.

LRMS: m/Z 409 (M+1)⁺.

Retention Time: 13 min.

δ(CDCl₃): 1.45 (t, 3H), 1.92 (s, 3H), 4.31 (q, 2H), 5.01 (s, 1H), 7.29 (m, 1H), 7.37 (m, 5H), 7.56 (m, 1H), 8.00 (s, 1H), 8.49 (s, 2H).

### Example 20

### 1-(Acetyloxy)ethyl 1-ethyl-6-oxo-3-phenyl-5-(pyridin-3-ylamino)-1,6-dihydropyridazine-4-carboxylate

Obtained as a solid (49%) from the title product of Preparation 28 and 1-chloroethyl acetate (Helv. Chim. Acta, 1978, 61, 192) following the experimental procedure of Example 17.

LRMS: m/Z 423 (M+1)⁺.

Retention Time: 14 min.

δ(DMSO-d₆): 0.81 (d, 3H), 1.34 (t, 3H), 1.85 (s, 3H), 4.18 (q, 2H), 5.87 (q, 1H), 7.32 (m, 3H), 7.39 (m, 3H), 7.51 (m, 1H), 8.33 (m, 1H), 8.39 (m, 1H), 9.33 (s, 1H).

### Examples 21-24

### 2-(Dimethylamino)-2-oxoethyl 1-ethyl-6-oxo-3-phenyl-5-(pyridin-3-ylamino)-1,6-dihydropyridazine-4-carboxylate

### Benzyl 1-ethyl-6-oxo-3-phenyl-5-(pyridin-3-ylamino)-1,6-dihydropyridazine-4-carboxylate

### [(2,2-Dimethylpropanoyl)oxy]methyl 1-ethyl-6-oxo-3-phenyl-5-(pyridin-3-ylamino)-1,6-dihydropyridazine-4-carboxylate

### 1-(Acetyloxy)-1-methylethyl 1-ethyl-6-oxo-3-phenyl-5-(pyridin-3-ylamino)-1,6-dihydropyridazine-4-carboxylate

The title compounds were synthesized from the title compound of Preparation 28 and the corresponding bromide or chloride following the procedure of Example 17. The ESI/MS data and HPLC retention times are summarized in Table 2.

TABLE 2

| | ESI/MS | Retention |
|---|---|---|
| EXAMPLE | m/e | Time (min) |
| 21 | 421 | 6.8* |
| 22 | 427 | 9.2* |
| 23 | 451 | 17 |
| 24 | 437 | 15 |

*Chromatographic method B

### Example 25

### Ethyl 1-ethyl-5-[(4-methylpyridin-3-yl)amino]-6-oxo-3-phenyl-1,6-dihydropyridazine-4-carboxylate

Obtained as a solid (20%) from the title compound of Preparation 4 and 4-methyl-3-bromopyridine following the experimental procedure of Example 15.

m.p. 166.0-167.2° C.

δ(DMSO-d₆): 0.66 (t, 3H), 1.33 (t, 3H), 2.19 (s, 3H), 3.01 (q, 2H), 4.16 (q, 2H), 7.26 (m, 3H), 7.33 (m, 3H), 8.16 (s, 1H), 8.26 (d, 1H), 8.90 (s, 1H).

### Example 26

### [(2,2-dimethylpropanoyl)oxy]methyl 1-ethyl-5-[(4-methylpyridin-3-yl)amino]-6-oxo-3-phenyl-1,6-dihydropyridazine-4-carboxylate

Obtained as a solid (73%) from the title product of Preparation 29 and chloromethyl pivalate following the experimental procedure of Example 17.

LRMS: m/Z 465 (M+1)⁺.

Retention Time: 17 min.

δ(DMSO-d₆): 1.012 (s, 9H), 1.34 (t, 3H), 2.24 (s, 3H), 4.18 (q, 2H), 4.68 (s, 2H), 7.32 (m, 6H), 8.24 (s, 1H), 8.32 (d, 1H), 9.07 (s, 1H).

### Example 27

### 1-[(Ethoxycarbonyl)oxy]ethyl 1-ethyl-5-[(4-methylpyridin-3-yl)amino]-6-oxo-3-phenyl-1,6-dihydropyridazine-4-carboxylate

Obtained as a solid (9%) from the title product of Preparation 29 and 1-chloroethyl carbonate (Preparation 48) following the experimental procedure of Example 17.

LRMS: m/Z 467 (M+1)⁺.

Retention Time: 16 min.

### Example 28

### 2-(Benzyloxy)-2-oxoethyl 1-ethyl-5-[(4-methylpyridin-3-yl)amino]-6-oxo-3-phenyl-1,6-dihydropyridazine-4-carboxylate

A mixture of the title compound of Preparation 6 (550 mg, 1.35 mmol), 4-methyl-3-bromopyridine (0.18 ml, 1.62 mmol), anhydrous cuprous iodide (26 mg, 0.13 mmol), N,N′-dimethylethylenediamine (29 μl, 0.27 mmol) and potassium carbonate (373 mg, 2.7 mmol) in dry dioxane (1.5 ml) was stirred under argon at 130° C. for 24 h. It was let to cool down and filtered. The precipitate was thoroughly washed with dichloromethane. Finally, solvent was removed under reduced pressure. The resulting residue was purified by flash column chromatography (SiO₂, dichloromethane-ethyl acetate) to yield the title product (100 mg, 15% yield).

m.p. 114.9-115.6° C.

δ(DMSO-d₆): 1.35 (t, 3H), 2.18 (s, 3H), 3.67 (s, 2H), 4.20 (q, 2H), 5.07 (s, 2H), 6.83 (m, 1H), 7.30 (m, 10H), 8.22 (m, 3H), 9.05 (m, 1H).

### Example 29

### 1-[(Ethoxycarbonyl)oxy]ethyl 1-ethyl-6-oxo-3-phenyl-5-(pyridin-3-ylamino)-1,6-dihydropyridazine-4-carboxylate

Obtained as a solid (47%) from the title product of Preparation 28 and 1-chloroethyl ethyl carbonate (Preparation 48) following the experimental procedure of Example 17.

LRMS: m/Z 453 (M+1)⁺.

Retention Time: 16 min.

δ(DMSO-d₆): 0.82 (d, 3H), 1.15 (t, 3H), 1.35 (t, 3H), 4.08 (q, 2H), 4.20 (q, 2H), 5.82 (q, 1H), 7.32 (m, 5H), 7.48 (m, 1H), 8.41 (m, 2H), 9.40 (s, 1H).

### Example 30

### Ethyl 1-ethyl-5-(isoquinolin-4-ylamino)-6-oxo-3-phenyl-1,6-dihydropyridazine-4-carboxylate

Obtained as a solid (34%) from the title compound of Preparation 4 and 4-bromoisoquinoline following the experimental procedure of Example 15.

LRMS: m/Z 415 (M+1)⁺.

Retention Time: 8.9* min. *Chromatographic method B

δ(CDCl₃): 0.46 (t, 3H), 1.43 (t, 3H), 3.10 (q, 2H), 4.36 (q, 2H), 7.36 (m, 5H), 7.78 (m, 3H), 8.16 (m, 4H).

### Example 31

### [(2,2-Dimethylpropanoyl)oxy]methyl 1-ethyl-5-(isoquinolin-4-ylamino)-6-oxo-3-phenyl-1,6-dihydropyridazine-4-carboxylate

Obtained as a solid (60%) from the title product of Preparation 30 and chloromethyl pivalate following the experimental procedure of Example 17.

LRMS: m/Z 501 (M+1)⁺.

Retention Time: 19 min.

δ(DMSO-d₆): 0.91 (s, 9H), 1.34 (t, 3H), 2.24 (s, 3H), 4.15 (s, 2H), 4.23 (q, 2H), 7.28 (m, 5H), 7.75 (t, 1H), 7.82 (t, 1H), 8.01 (d, 1H), 8.22 (d, 1H), 8.31 (s, 1H), 9.42 (s, 1H), 9.44 (s, 1H).

### Example 32

### 1-(Acetyloxy)ethyl 1-ethyl-5-(isoquinolin-4-ylamino)-6-oxo-3-phenyl-1,6-dihydropyridazine-4-carboxylate

Obtained as a solid (50%) from the title product of Preparation 30 and 1-chloroethyl ethyl acetate (Helv. Chim. Acta, 1978, 61, 192) following the experimental procedure of Example 17.

LRMS: m/Z 473 (M+1)⁺.

Retention Time: 16 min.

δ(DMSO-d₆): 0.40 (d, 3H), 1.38 (t, 3H), 1.71 (s, 3H), 4.23 (q, 2H), 5.39 (q, 1H), 7.27 (m, 2H), 7.35 (m, 3H), 7.73 (t, 1H), 7.84 (t, 1H), 8.00 (d, 1H), 8.20 (d, 1H), 8.30 (s, 1H), 9.23 (s, 1H), 9.43 (s, 1H).

### Example 33

### ({[1-Ethyl-6-oxo-3-phenyl-5-(pyridin-3-ylamino)-1,6-dihydropyridazin-4-yl]carbonyl}oxy)acetic acid

A mixture of the title compound of Example 16 (57 mg, 0.1 mmol) and 10% palladium on charcoal (6 mg) in THF (5 ml) was shaken under hydrogen at room temperature and atmospheric pressure for 1 h. The catalyst was filtered off and the solvent was removed under reduced pressure to yield the title compound that was purified by preparative HPLC/MS.

LRMS: m/Z 395 (M+1)⁺.

Retention Time: 11 min.

### Example 34

### Ethyl 1-ethyl-3-(3-methylphenyl)-6-oxo-5-(pyridin-3-ylamino)-1,6-dihydropyridazine-4-carboxylate

Obtained as a solid (42%) from the title compound of Preparation 10 and 3-bromopyridine following the experimental procedure of Example 15.

m.p.: 130.8-131.9° C.

δ(CDCl₃): 0.76 (t, 3H), 1.45 (t, 3H), 2.35 (s, 3H), 3.42 (q, 2H), 4.31 (q, 2H), 7.22 (m, 5H), 7.46 (m, 1H), 7.90 (s, 1H), 8.45 (m, 1H), 8.47 (m, 1H).

### Example 35

### [(2,2-Dimethylpropanoyl)oxy]methyl 1-ethyl-3-(3-methylphenyl)-6-oxo-5-(pyridin-3-ylamino)-1,6-dihydropyridazine-4-carboxylate

Obtained as a solid (73%) from the title product of Preparation 31 and chloromethyl pivalate following the experimental procedure of Example 17.

LRMS: m/Z 465 (M+1)⁺.

Retention Time: 17 min.

δ (CDCl₃): 1.01 (s, 9H), 1.43 (t, 3H), 2.38 (s, 3H), 4.28 (q, 2H), 4.98 (s, 2H), 7.22 (m, 5H), 7.46 (m, 1H), 8.17 (s, 1H), 8.44 (m, 2H).

### Example 36

### Ethyl 1-ethyl-3-(3-fluorophenyl)-6-oxo-5-(pyridin-3-ylamino)-1,6-dihydropyridazine-4-carboxylate

Obtained as a solid (32%) from the title compound of Preparation 13 and 3-bromopyridine following the experimental procedure of Example 15.

LRMS: m/Z 383 (M+1)⁺.

Retention Time: 14 min.

δ(CDCl₃): 0.80 (t, 3H), 1.45 (t, 3H), 3.46 (q, 2H), 4.31 (q, 2H), 7.11 (m, 3H), 7.32 (m, 2H), 7.47 (m, 1H), 7.97 (s, 1H), 8.45 (m, 2H).

### Example 37

### (Butyryloxy)methyl 1-ethyl-3-(3-fluorophenyl)-6-oxo-5-(pyridin-3-ylamino)-1,6-dihydropyridazine-4-carboxylate

Obtained as a solid (36%) from the title product of Preparation 32 and chloromethyl butyrate following the experimental procedure of Example 17.

LRMS: m/Z 455 (M+1)⁺.

Retention Time: 16 min.

δ(CDCl₃): 0.90 (t, 3H), 1.45 (t, 3H), 1.50 (m, 2H), 2.18 (t, 2H), 4.30 (q, 2H), 5.03 (s, 2H), 7.08 (m, 2H), 7.17 (m, 1H), 7.31 (m, 2H), 7.47 (m, 1H), 8.06 (s, 1H), 8.49 (m, 2H).

### Example 38

### Ethyl 1-ethyl-3-(4-fluorophenyl)-6-oxo-5-(pyridin-3-ylamino)-1,6-dihydropyridazine-4-carboxylate

Obtained as a solid (50%) from the title compound of Preparation 16a and 3-bromopyridine following the experimental procedure of Example 15.

LRMS: m/Z 383 (M+1)⁺.

Retention Time: 14 min.

δ(CDCl₃): 0.80 (t, 3H), 1.45 (t, 3H), 3.44 (q, 2H), 4.30 (q, 2H), 7.06 (t, 2H), 7.28 (m, 1H), 7.34 (m, 2H), 7.46 (m, 1H), 7.94 (s, 1H), 8.47 (m, 2H).

### Example 39

### (Butyryloxy)methyl 1-ethyl-3-(4-fluorophenyl)-6-oxo-5-(pyridin-3-ylamino)-1,6-dihydropyridazine-4-carboxylate

Obtained as a solid (19%) from the title product of Preparation 33 and chloromethyl butyrate following the experimental procedure of Example 17.

LRMS: m/Z 455 (M+1)⁺.

Retention Time: 16 min.

δ(CDCl₃): 0.91 (t, 3H), 1.44 (t, 3H), 1.50 (m, 2H), 2.15 (t, 2H), 4.30 (q, 2H), 5.03 (s, 2H), 7.05 (m, 2H), 7.31 (m, 3H), 7.47 (m, 1H), 8.01 (s, 1H), 8.49 (m, 2H).

### Example 40

### Ethyl 5-[(2-chloropyridin-3-yl)amino]-1-ethyl-6-oxo-3-phenyl-1,6-dihydropyridazine-4-carboxylate

Obtained as a solid (27%) from the title product of Preparation 4 and 2-chloropyridin-3-boronic acid following the experimental procedure of Preparation 27.

LRMS: m/Z 399 (M+1)⁺.

Retention Time: 15 min.

δ(CDCl₃): 0.89 (t, 3H), 1.42 (t, 3H), 3.55 (q, 2H), 4.32 (q, 2H), 7.18 (m, 1H), 7.38 (m, 5H), 7.42 (d, 1H), 8.01 (s, 1H), 8.22 (m, 1H).

### Example 41

### Methyl 1-ethyl-6-oxo-3-phenyl-5-(pyridin-3-ylamino)-1,6-dihydropyridazine-4-carboxylate

Obtained as a solid (9%) from the title product of Preparation 16b and 3-bromopyridine following the experimental procedure of Example 15.

m.p. 180.0-180.6° C.

δ(DMSO-d₆): 1.34 (t, 3H), 3.34 (s, 3H), 4.18 (q, 2H), 7.34 (m, 6H), 7.47 (m, 1H), 8.34 (m, 2H), 9.27 (s, 1H).

### Example 42

### Methyl 1-ethyl-6-oxo-3-phenyl-5-(quinolin-5-ylamino)-1,6-dihydropyridazine-4-carboxylate

Obtained as a solid (56%) from the title product of Preparation 16b and 5-quinolineboronic acid following the experimental procedure of Preparation 27.

LRMS: m/Z 401 (M+1)⁺.

Retention Time: 14 min.

δ(CDCl₃): 1.61 (t, 3H), 2.69 (s, 3H), 4.48 (q, 2H), 7.45 (m, 6H), 7.61 (m, 1H), 7.74 (t, 1H), 8.14 (m, 2H), 8.62 (d, 1H), 9.11 (m, 1H).

### Example 43

### Ethyl 1-ethyl-6-oxo-5-(pyridin-3-ylamino)-3-(2-thienyl)-1,6-dihydropyridazine-4-carboxylate

In a pressure reactor were placed the title product of Preparation 21 (1.0 g, 3.41 mmol), 3-bromopyridine (0.65 g, 4.09 mmol), copper(I) iodide (65 mg, 0.34 mmol), potassium carbonate (0.99 g, 7.2 mmol), N,N-dimethylethylenediamine (60 mg, 0.68 mmol) and dioxane (6 ml). This mixture was heated at 120° C. for 48 h under argon. Once at room temperature, the reaction mixture was filtered and solvent was removed under reduced pressure. The residue was purified by flash chromatography (CH2Cl2 to CH2Cl2:MeOH 98:2 as eluent). 0.56 g (44% yield) of the desired final product as a white-off solid were isolated.

m.p.: 123.8-124.6° C.

δ(DMSO-d₆): (t, J=7.0 Hz, 3H) 1.3 (t, J=7.3 Hz, 3H) 3.3 (m, 3H) 4.2 (q, J=7.3 Hz, 2H) 7.0 (d, J=3.7 Hz, 1H) 7.0 (m, 1H) 7.4 (dd, J=8.1, 4.8 Hz, 1H) 7.5 (m, 1H) 7.6 (d, J=4.1 Hz, 1H) 8.4 (m, 1H) 9.2 (s, 1H)

### Example 44

### 2-(Acetyloxy)ethyl 1-ethyl-6-oxo-5-(pyridin-3-ylamino)-3-(2-thienyl)-1,6-dihydropyridazine-4-carboxylate

A mixture of the title product of Preparation 34 (0.1 g, 0.29 mmol), potassium carbonate (0.12 g, 0.87 mmol) and 2-bromoethylacetate (53 mg, 0.32 mmol) in dimethylformamide (3 ml) were heated at 50° C. for 4 h. Once at room temperature, it was poured into water and extracted with ethyl acetate. After successively washing of the organic phase with aqueous 4% NaHCO3, water and brine, it was dried over magnesium sulfate, filtered and evaporated under reduced pressure. 80 mg (67% yield) of the desired final product were obtained.

m.p. 137.9-139.5° C.

δ(DMSO-d₆): 1.3 (t, J=7.3 Hz, 3H) 2.0 (m, 3H) 3.5 (m, 2H) 3.9 (m, 2H) 4.2 (q, J=7.0 Hz, 2H) 7.0 (m, 2H) 7.4 (s, 1H) 7.6 (m, 4H) 9.2 (s, 1H)

### Example 45

### 2-[(tert-Butoxycarbonyl)amino]ethyl 1-ethyl-6-oxo-5-(pyridin-3-ylamino)-3-(2-thienyl)-1,6-dihydropyridazine-4-carboxylate

Obtained as a solid (29%) from the title product of Preparation 34 and 2-(tert-butoxycarbonylamino)ethyl bromide following the experimental procedure described in Example 44.

m.p.: 151.4-153.6° C.

δ(DMSO-d₆): 1.3 (m, 12H) 2.9 (m, 2H) 3.3 (t, J=5.8 Hz, 2H) 4.2 (q, J=7.0 Hz, 2H) 6.8 (s, 1H) 7.0 (m, 2H) 7.4 (dd, J=8.1, 4.8 Hz, 1H) 7.6 (m, 2H) 8.4 (m, 2H) 9.2 (s, 1H).

### Example 46

### 2-Ethoxy-2-oxoethyl 1-ethyl-6-oxo-5-(pyridin-3-ylamino)-3-(2-thienyl)-1,6-dihydropyridazine-4-carboxylate

Obtained as a solid (68%) from the title product of Preparation 34 and ethyl bromoacetate following the experimental procedure described in Example 44.

m.p.: 125.8-127.0° C.

δ(DMSO-d₆): 1.1 (t, J=7.0 Hz, 3H) 1.3 (t, J=7.3 Hz, 3H) 3.8 (s, 2H) 4.1 (q, J=7.0 Hz, 2H) 4.2 (q, J=7.3 Hz, 2H) 7.0 (m, 1H) 7.2 (d, J=3.7 Hz, 1H) 7.4 (dd, J=8.1, 4.8 Hz, 1H) 7.5 (d, J=8.3 Hz, 1H) 7.6 (d, J=5.0 Hz, 1H) 8.4 (m, 2H) 9.3 (s, 1H)

### Example 47

### 2-(Benzyloxy)-2-oxoethyl 1-ethyl-6-oxo-5-(pyridin-3-ylamino)-3-(2-thienyl)-1,6-dihydropyridazine-4-carboxylate

Obtained as a solid (77%) from the title product of Preparation 34 and benzyl bromoacetate following the experimental procedure described in Example 44.

m.p.: 110.9-111.4° C.

δ(DMSO-d₆): 1.3 (t, J=7.0 Hz, 3H) 3.9 (s, 2H) 4.2 (t, J=7.3 Hz, 2H) 5.1 (s, 2H) 6.9 (m, 1H) 7.2 (d, J=3.7 Hz, 1H) 7.3 (m, 6H) 7.5 (d, J=8.3 Hz, 1H) 7.6 (d, J=5.4 Hz, 1H) 8.4 (s, 2H) 9.3 (s, 1H)

### Example 48

### Benzyl 1-ethyl-6-oxo-5-(pyridin-3-ylamino)-3-(2-thienyl)-1,6-dihydropyridazine-4-carboxylate

Obtained as a solid (42%) from the title product of Preparation 34 and benzyl bromide following the experimental procedure described in Example 44.

m.p.: 143.1-144.9° C.

δ(DMSO-d₆): 1.3 (t, J=7.3 Hz, 3H) 4.2 (q, J=7.0 Hz, 2H) 4.3 (s, 2H) 6.9 (d, J=3.7 Hz, 1H) 6.9 (m, 1H) 7.0 (m, 2H) 7.3 (m, 4H) 7.6 (m, 2H) 8.4 (dd, J=10.6, 2.7 Hz, 2H) 9.2 (s, 1H)

### Example 49

### Ethyl 1-ethyl-5-(4-methylpyridin-3-ylamino)-6-oxo-3-thiophen-2-yl-1,6-dihydropyridazin-4-carboxylate

Obtained as a solid (41%) from the title product of Preparation 21 and 4-methyl-3-bromopyridine following the experimental procedure described in Example 43.

m.p.: 159.5-160.2° C.

δ(DMSO-d₆): 0.87 (t, 3H), 1.35 (t, 3H), 2.21 (s, 3H), 3.21 (q, 2H), 4.17 (q, 2H), 6.94 (d, 1H), 7.01 (m, 1H), 7.30 (d, 1H), 7.58 (d, 1H), 8.24 (s, 1H), 8.31 (d, 1H), 8.90 (s, 1H).

### Example 50

### 2-(Acetyloxy)ethyl 1-ethyl-5-[(4-methylpyridin-3-yl)amino]-6-oxo-3-(2-thienyl)-1,6-dihydropyridazine-4-carboxylate

A mixture of the title product of Preparation 35 (0.15 g, 0.42 mmol), potassium carbonate (0.17 g, 12.63 mmol) and 2-bromoethylacetate (77.2 mg, 0.46 mmol) in dimethylformamide (4 ml) were heated at 50° C. for 4 h. Once at room temperature, solvent was evaporated under reduced pressure and the residue was purified by flash chromatography (CH2Cl2:MeOH 99:1 as eluent). 0.13 g (68%) of the desired final product were isolated.

m.p.: 161.0-161.6° C.

δ(DMSO-d₆): 1.4 (t, J=7.0 Hz, 3H) 1.9 (s, 3H) 2.2 (s, 2H) 3.4 (m, 3H) 3.9 (m, 2H) 4.2 (q, J=7.0 Hz, 2H) 7.0 (m, 2H) 7.3 (d, J=5.0 Hz, 1H) 7.6 (d, J=5.0 Hz, 1H) 8.3 (s, 1H) 8.3 (d, J=5.0 Hz, 1H) 9.0 (s, 1H)

### Example 51

### 2-[(tert-Butoxycarbonyl)amino]ethyl 1-ethyl-5-[(4-methylpyridin-3-yl)amino]-6-oxo-3-(2-thienyl)-1,6-dihydropyridazine-4-carboxylate

Obtained as a solid (62%) from the title product of Preparation 35 and 2-(tert-butoxycarbonylamino)ethyl bromide following the experimental procedure described in Example 50.

m.p.: 181.8-182.4° C.

δ(DMSO-d₆): 1.3 (m, 12H) 2.2 (s, 3H) 2.9 (d, J=5.8 Hz, 2H) 3.1 (t, J=6.0 Hz, 2H) 4.2 (q, J=7.2 Hz, 2H) 6.8 (s, 1H) 7.0 (m, 2H) 7.3 (d, J=5.0 Hz, 1H) 7.6 (d, J=5.0 Hz, 1H) 8.2 (s, 1H) 8.3 (d, J=5.0 Hz, 1H) 8.9 (s, 1H)

### Example 52

### Ethyl 1-ethyl-5-(isoquinolin-4-ylamino)-6-oxo-3-(2-thienyl)-1,6-dihydropyridazine-4-carboxylate

Obtained as a solid (32%) from the title product of Preparation 21 and 4-bromoisoquinoline following the experimental procedure described in Example 43.

m.p. 176.3-177.0° C.

δ(DMSO-d₆): (t, J=7.0 Hz, 3H) 1.4 (m, 3H) 2.8 (d, J=7.0 Hz, 2H) 4.2 (q, J=7.2 Hz, 2H) 6.9 (d, J=3.7 Hz, 1H) 7.0 (m, 1H) 7.6 (d, J=4.1 Hz, 1H) 7.7 (t, J=7.5 Hz, 1H) 7.8 (t, J=7.0 Hz, 1H) 8.0 (d, J=9.5 Hz, 1H) 8.2 (d, J=7.9 Hz, 1H) 8.3 (s, 1H) 9.3 (d, J=14.5 Hz, 2H).

### Example 53

### 2-(Acetyloxy)ethyl 1-ethyl-5-(isoquinolin-4-ylamino)-6-oxo-3-(2-thienyl)-1,6-dihydropyridazine-4-carboxylate

A mixture of the title product of Preparation 36 (0.15 g, 0.38 mmol), potassium carbonate (0.15 g, 1.08 mmol) and 2-bromoethylacetate (66.8 mg, 0.40 mmol) in dimethylformamide (3 ml) were heated at 50° C. for 4 h. Once at room temperature, solvent was evaporated under reduced pressure and the residue was purified by flash chromatography (CH2Cl2:MeOH 99:1 as eluent). 0.13 g (72% yield) of the desired final product were isolated.

m.p.: 155.2-156.7° C.

δ(DMSO-d₆): 1.4 (t, J=7.0 Hz, 3H) 1.9 (s, 3H) 3.0 (s, 2H) 3.5 (s, 2H) 4.2 (q, J=6.6 Hz, 2H) 6.9 (s, 1H) 7.0 (s, 1H) 7.6 (d, J=5.0 Hz, 1H) 7.7 (d, J=7.5 Hz, 1H) 7.8 (m, 1H) 7.9 (d, J=8.3 Hz, 1H) 8.2 (d, J=8.3 Hz, 1H) 8.3 (s, 1H) 9.2 (s, 1H) 9.4 (s, 1H).

### Example 54

### 2-[(tert-Butoxycarbonyl)amino]ethyl 1-ethyl-5-(isoquinolin-4-ylamino)-6-oxo-3-(2-thienyl)-1,6-dihydropyridazine-4-carboxylate

Obtained as a solid (50%) from the title product of Preparation 36 and 2-(tert-butoxycarbonylamino)ethyl bromide following the experimental procedure described in Example 53.

m.p.: 146.1-147.5° C.

δ(DMSO-d₆): 1.3 (s, 9H) 1.4 (t, J=7.3 Hz, 3H) 2.8 (s, 2H) 3.3 (m, 2H) 4.2 (m, 2H) 6.6 (s, 1H) 6.9 (d, J=3.3 Hz, 1H) 6.9 (m, 1H) 7.6 (d, J=4.1 Hz, 1H) 7.7 (d, J=7.9 Hz, 1H) 7.8 (t, J=7.0 Hz, 1H) 7.9 (d, J=7.9 Hz, 1H) 8.2 (d, J=8.3 Hz, 1H) 8.3 (s, 1H) 9.2 (s, 1H) 9.3 (s, 1H)

### Example 55

### 2-Ethoxy-2-oxoethyl 1-ethyl-5-(isoquinolin-4-ylamino)-6-oxo-3-(2-thienyl)-1,6-dihydropyridazine-4-carboxylate

Obtained as a solid (78%) from the title product of Preparation 36 and ethyl bromoacetate following the experimental procedure described in Example 53.

m.p. 141.6-142.3° C.

δ(DMSO-d₆): 1.1 (t, J=7.0 Hz, 3H) 1.4 (t, J=7.3 Hz, 3H) 3.2 (s, 2H) 3.9 (q, J=7.3 Hz, 2H) 4.2 (q, J=7.0 Hz, 2H) 6.9 (m, 1H) 7.1 (d, J=3.7 Hz, 1H) 7.5 (d, J=6.2 Hz, 1H) 7.7 (t, J=7.7 Hz, 1H) 7.8 (t, J=7.7 Hz, 1H) 8.0 (d, J=8.3 Hz, 1H) 8.2 (d, J=8.3 Hz, 1H) 8.3 (s, 1H) 9.3 (s, 1H) 9.4 (s, 1H)

### Example 56

### 2-(Benzyloxy)-2-oxoethyl 1-ethyl-5-(isoquinolin-4-ylamino)-6-oxo-3-(2-thienyl)-1,6-dihydropyridazine-4-carboxylate

Obtained as a solid (57%) from the title product of Preparation 36 and benzyl bromoacetate following the experimental procedure described in Example 53.

m.p.: 148.2-149.6° C.

δ(DMSO-d₆): 1.4 (m, 3H) 3.3 (s, 2H) 4.2 (q, J=7.0 Hz, 2H) 5.0 (s, 2H) 6.9 (m, 1H) 7.1 (d, J=3.7 Hz, 1H) 7.3 (m, 2H) 7.4 (m, 3H) 7.5 (d, J=4.1 Hz, 1H) 7.7 (t, J=7.0 Hz, 1H) 7.8 (t, J=7.0 Hz, 1H) 7.9 (d, J=7.5 Hz, 1H) 8.2 (d, J=7.9 Hz, 1H) 8.3 (s, 1H) 9.3 (s, 1H) 9.4 (s, 1H).

### Example 57

### Benzyl 1-ethyl-5-(isoquinolin-4-ylamino)-6-oxo-3-(2-thienyl)-1,6-dihydropyridazine-4-carboxylate

Obtained as a solid (44%) from the title product of Preparation 36 and benzyl bromide following the experimental procedure described in Example 53.

m.p.: 195.7-196.7° C.

δ(DMSO-d₆): 1.4 (t, J=7.0 Hz, 3H) 3.8 (s, 2H) 4.2 (q, J=7.3 Hz, 2H) 6.7 (m, 2H) 6.9 (m, 1H) 7.2 (m, 4H) 7.5 (d, J=5.0 Hz, 1H) 7.8 (m, 2H) 8.0 (d, J=8.3 Hz, 1H) 8.2 (d, J=7.9 Hz, 1H) 8.4 (s, 1H) 9.3 (d, J=13.7 Hz, 2H).

### Example 58

### Ethyl 1-ethyl-5-(isoquinolin-4-ylamino)-6-oxo-3-(3-thienyl)-1,6-dihydropyridazine-4-carboxylate

Obtained as a solid (39%) from the title product of Preparation 26 and 4-bromoisoquinoline following the experimental procedure described in Example 50.

m.p.: 140.2-141.8° C.

δ(DMSO-d₆): 0.4 (t, J=7.0 Hz, 3H) 1.4 (t, J=7.3 Hz, 3H) 2.8 (q, J=7.0 Hz, 2H) 4.2 (q, J=7.0 Hz, 2H) 7.0 (d, J=5.0 Hz, 1H) 7.4 (d, J=2.9 Hz, 1H) 7.5 (dd, J=5.0, 2.9 Hz, 1H) 7.7 (m, 1H) 7.8 (t, J=7.7 Hz, 1H) 8.0 (d, J=8.3 Hz, 1H) 8.2 (d, J=8.3 Hz, 1H) 8.3 (s, 1H) 9.2 (s, 2H).

### Example 59

### 2-(Acetyloxy)ethyl 1-ethyl-5-(isoquinolin-4-ylamino)-6-oxo-3-(3-thienyl)-1,6-dihydropyridazine-4-carboxylate

A mixture of the title product of Preparation 37 (0.15 g, 0.38 mmol), potassium carbonate (0.15 g, 1.08 mmol) and 2-bromoethylacetate (66.8 mg, 0.40 mmol) in dimethylformamide (3 ml) were heated at 50° C. for 4 h. Once at room temperature, solvent was evaporated under reduced pressure and the residue was purified by flash chromatography (CH2Cl2 to CH2Cl2:MeOH 98:2 as eluent). 0.15 g of the desired final product were isolated (63% yield).

m.p. 175.8-177.1° C.

δ(DMSO-d₆): 1.4 (t, J=7.0 Hz, 3H) 1.9 (s, 3H) 2.9 (s, 2H) 3.5 (m, 2H) 4.2 (q, J=7.0 Hz, 2H) 7.1 (d, J=5.0 Hz, 1H) 7.4 (d, J=1.7 Hz, 1H) 7.5 (dd, J=5.0, 2.9 Hz, 1H) 7.7 (d, J=7.0 Hz, 1H) 7.8 (t, J=7.0 Hz, 1H) 7.9 (d, J=8.7 Hz, 1H) 8.2 (d, J=8.3 Hz, 1H) 8.3 (s, 1H) 9.2 (s, 1H) 9.3 (s, 1H).

### Example 60

### 2-[(tert-Butoxycarbonyl)amino]ethyl 1-ethyl-5-(isoquinolin-4-ylamino)-6-oxo-3-(3-thienyl)-1,6-dihydropyridazine-4-carboxylate

Obtained as a solid (65%) from the title product of Preparation 37 and 2-(tert-butoxycarbonylamino)ethyl bromide following the experimental procedure described in Example 59.

m.p.: 122.2-123.8° C.

δ(DMSO-d₆): 1.3 (m, 12H) 2.4 (d, J=5.4 Hz, 2H) 2.7 (d, J=14.5 Hz, 2H) 6.6 (s, 2H) 7.0 (d, J=4.6 Hz, 1H) 7.4 (s, 1H) 7.5 (m, 1H) 7.7 (d, J=7.5 Hz, 1H) 7.8 (d, J=7.5 Hz, 1H) 7.9 (d, J=7.9 Hz, 1H) 8.2 (d, J=7.9 Hz, 1H) 8.2 (d, J=7.9 Hz, 1H) 8.3 (s, 1H) 9.2 (s, 1H) 9.3 (s, 1H)

### Example 61

### 2-Ethoxy-2-oxoethyl 1-ethyl-5-(isoquinolin-4-ylamino)-6-oxo-3-(3-thienyl)-1,6-dihydropyridazine-4-carboxylate

Obtained as a solid (50%) from the title product of Preparation 37 and ethyl bromoacetate following the experimental procedure described in Example 59.

m.p. 161.4-161.9° C.

δ(DMSO-d₆): 1.1 (t, J=7.1 Hz, 3H) 1.4 (t, J=7.1 Hz, 3H) 3.1 (s, 2H) 3.9 (q, J=7.1 Hz, 2H) 4.2 (t, J=7.1 Hz, 2H) 7.2 (dd, J=4.9, 1.4 Hz, 1H) 7.5 (m, 2H) 7.8 (m, 2H) 8.0 (m, 1H) 8.2 (d, J=8.0 Hz, 1H) 8.3 (s, 1H) 9.3 (s, 1H) 9.4 (s, 1H)

### Example 62

### 2-(Benzyloxy)-2-oxoethyl 1-ethyl-5-(isoquinolin-4-ylamino)-6-oxo-3-(3-thienyl)-1,6-dihydropyridazine-4-carboxylate

Obtained as a solid (49%) from the title product of Preparation 37 and benzyl bromoacetate following the experimental procedure described in Example 59.

m.p.: 150.0-150.7° C.

δ(DMSO-d₆): 1.4 (t, J=7.1 Hz, 3H) 3.2 (s, 2H) 4.2 (q, J=6.9 Hz, 2H) 5.0 (s, 2H) 7.2 (dd, J=4.9, 1.4 Hz, 2H) 7.2 (dd, J=4.9, 1.4 Hz, 1H) 7.3 (m, 2H) 7.5 (m, 3H) 7.7 (m, 1H) 7.8 (m, 1H) 8.0 (d, J=8.5 Hz, 1H) 8.2 (d, J=8.0 Hz, 1H) 8.3 (s, 1H) 9.2 (s, 1H) 9.4 (s, 1H)

### Example 63

### 4-Fluorobenzyl 1-ethyl-5-(isoquinolin-4-ylamino)-6-oxo-3-(2-thienyl)-1,6-dihydropyridazine-4-carboxylate

Obtained as a solid (30%) from the title product of Preparation 36 and 4-fluorobenzyl bromide following the experimental procedure described in Example 59.

m.p.: 196.8-197.4° C.

δ(DMSO-d₆): 1.4 (t, J=7.1 Hz, 3H) 3.8 (s, 2H) 4.2 (q, J=7.2 Hz, 2H) 6.8 (m, 3H) 6.9 (dd, J=5.2, 3.6 Hz, 1H) 7.0 (m, 2H) 7.6 (dd, J=5.2, 1.1 Hz, 1H) 7.8 (m, 2H) 8.0 (d, J=8.2 Hz, 1H) 8.2 (d, J=7.4 Hz, 1H) 8.4 (s, 1H) 9.3 (s, 1H) 9.4 (s, 1H)

### Example 64

### 4-(Methoxycarbonyl)benzyl 1-ethyl-5-(isoquinolin-4-ylamino)-6-oxo-3-(2-thienyl)-1,6-dihydropyridazine-4-carboxylate

Obtained as a solid (91%) from the title product of Preparation 36 and methyl 4-bromomethylbenzoate following the experimental procedure described in Example 59.

m.p.: 177.0-177.4° C.

δ(DMSO-d₆): 1.4 (t, J=7.1 Hz, 3H) 3.8 (s, 3H) 3.9 (s, 2H) 4.2 (q, J=7.3 Hz, 2H) 6.8 (m, 5H) 7.6 (dd, J=5.2, 1.1 Hz, 1H) 7.8 (m, 3H) 8.0 (d, J=8.5 Hz, 1H) 8.2 (d, J=8.0 Hz, 1H) 8.4 (s, 1H) 9.3 (s, 1H) 9.4 (s, 1H)

### Example 65

### Ethyl 1-ethyl-3-(4-methylphenyl)-6-oxo-5-(pyridin-3-ylamino)-1,6-dihydropyridazine-4-carboxylate

A stirred mixture of the title compound of Preparation 42 (800 mg, 2.66 mmol), 3-bromopyridine (504 mg, 3.19 mmol), anhydrous copper(I) iodide (51 mg, 0.266 mmol), N,N′-dimethylethylenediamine (47 mg, 0.531 mmol) and potassium carbonate (733 mg, 5.31 mmol) in anhydrous dioxane (8 ml) was heated in a sealed tube at 135° C. overnight. The reaction mixture was filtered through a pad of Celite®, the solvent was removed under reduced pressure and the residue purified by column chromatography (Biotage® cartridge CH2Cl2/EtOAc 50:50 to 0:100) to give the title compound as a brown solid (440 mg, 44% yield).

LRMS (m/z): 379 (M+1)⁺.

Retention Time: 15 min.

δ(DMSO-d6): 0.72 (t, 3H), 1.34 (t, 3H), 2.31 (s, 3H), 3.24 (q, 2H), 4.16 (q, 2H), 7.19 (s, 4H), 7.32 (m, 1H), 7.48 (d, 1H), 8.32 (d, 1H), 8.36 (s, 1H), 9.17 (s, 1H).

### Example 66

### Ethyl 1-ethyl-5-(isoquinolin-4-ylamino)-3-(4-methylphenyl)-6-oxo-1,6-dihydropyridazine-4-carboxylate

Obtained as a solid (45%) from the title compound of Preparation 42 and 4-bromoisoquinoline following the procedure of Example 65.

δ(DMSO-d6): 0.34 (t, 3H), 1.38 (t, 3H), 2.27 (s, 3H), 2.70 (q, 2H), 4.22 (q, 2H), 7.13 (s, 4H), 7.73 (t, 1H), 7.82 (t, 1H), 7.99 (d, 1H), 8.16 (d, 1H), 8.27 (s, 1H), 9.20 (s, 1H), 9.24 (s, 1H).

LRMS (m/z): 429 (M+1)⁺.

Retention Time: 16 min.

### Example 67

### Ethyl 1-ethyl-3-(4-methylphenyl)-5-[(4-methylpyridin-3-yl)amino]-6-oxo-1,6-dihydropyridazine-4-carboxylate

Obtained as a solid (53%) from the title compound of Preparation 42 and 4-methyl-3-bromopyridine following the procedure of Example 65.

δ(DMSO-d6): 0.72 (t, 3H), 1.34 (t, 3H), 2.21 (s, 3H), 2.30 (s, 3H), 3.10 (q, 2H), 4.18 (q, 2H), 7.17 (s, 4H), 7.28 (d, 1H), 8.19 (s, 1H), 8.28 (d, 1H), 8.85 (s, 1H).

LRMS (m/z): 393 (M+1)⁺.

Retention Time: 15 min.

### Example 68

### [(2,2-Dimethylpropanoyl)oxy]methyl 1-ethyl-3-(4-methylphenyl)-6-oxo-5-(pyridin-3-ylamino)-1,6-dihydropyridazine-4-carboxylate

To a solution of the title compound of Preparation 43 (124 mg, 0.35 mmol) in DMF (2.5 ml) chloromethyl pivalate (63 mg, 0.42 mmol) and diisopropylethylamine (58 mg, 0.45 mmol) were added and the reaction was stirred at 60° C. for 3 hours. The mixture was poured into water (50 ml) and extracted with ethyl acetate (20 ml×3). The combined organic phases were washed with brine, dried over anhydrous sodium sulfate and evaporated under reduced pressure to afford an oil which was purified by column chromatography (Biotage® cartridge CH2Cl2/EtOAc 50:50 to 0:100) to give the title compound as a solid (73 mg, 45% yield).

LRMS (m/z): 465 (M+1)⁺.

Retention Time: 17 min.

δ(DMSO-d6): 1.01 (s, 9H), 1.33 (t, 3H), 2.30 (s, 3H), 4.17 (q, 2H), 4.82 (s, 2H), 7.16 (d, 2H), 7.18 (d, 2H), 7.35 (m, 1H), 7.52 (d, 1H), 8.36 (d, 1H), 8.38 (s, 1H), 9.33 (s, 1H).

### Example 69

### [(2,2-Dimethylpropanoyl)oxy]methyl 1-ethyl-5-(isoquinolin-4-ylamino)-3-(4-methylphenyl)-6-oxo-1,6-dihydropyridazine-4-carboxylate

Obtained as a solid (41%) from the title compound of Preparation 44 following the procedure of Example 68.

LRMS (m/z): 515 (M+1)⁺.

Retention Time: 18 min.

δ(DMSO-d6): 0.90 (s, 9H), 1.38 (t, 3H), 2.26 (s, 3H), 4.18 (s, 2H), 4.21 (q, 2H), 7.10 (d, 2H), 7.15 (d, 2H), 7.75 (t, 1H), 7.85 (t, 1H), 7.99 (d, 1H), 8.19 (d, 1H), 8.31 (s, 1H), 9.24 (s, 1H), 9.41 (s, 1H).

### Example 70

### [(2,2-Dimethylpropanoyl)oxy]methyl 1-ethyl-3-(4-methylphenyl)-5-[(4-methylpyridin-3-yl)amino]-6-oxo-1,6-dihydropyridazine-4-carboxylate

Obtained as a solid (32%) from the title compound of Preparation 45 following the procedure of Example 68.

LRMS (m/z): 479 (M+1)⁺.

Retention Time: 18 min.

δ(DMSO-d6): 1.00 (s, 9H), 1.34 (t, 3H), 2.23 (s, 3H), 2.29 (s, 3H), 4.17 (q, 2H), 4.69 (s, 2H), 7.14 (d, 2H), 7.18 (d, 2H), 7.32 (d, 1H), 8.23 (s, 1H), 8.32 (d, 1H), 9.04 (s, 1H).

### Example 71

### 1-[(Isopropoxycarbonyl)oxy]ethyl 1-ethyl-6-oxo-3-phenyl-5-(pyridin-3-ylamino)-1,6-dihydropyridazine-4-carboxylate

To a solution of the title compound of Preparation 28 (100 mg, 0.298 mmole) and the title compound from Preparation 46 (92 mg, 0.356 mmol) in 2 ml of DMF potassium carbonate (54 mg, 0.387 mmole) was added and the mixture was stirred at 60° C. for 3 h. The reaction mixture was diluted with ethyl acetate (100 ml) washed with water, brine and dried over anhydrous sodium sulphate. Removal of the solvent under reduced pressure afforded 300 mg of a brown oil which was purified by column cromatography (CH2Cl2/EtOAc 1:1) to give the title compound as a yellow oil. It was crystallized in diisopropyl ether (10 ml) to afford a white solid (45 mg, 57% yield).

LRMS (m/z): 467 (M+1)⁺.

Retention Time: 16 min.

δ(DMSO-d₆): 0.84 (d, 3H), 1.19 (t, 6H), 1.34 (t, 3H), 4.17 (q, 2H), 4.67 (m, 1H), 5.80 (q, 1H), 7.28-7.40 (m, 6H), 7.49 (d, 1H), 8.31 (d, 1H), 8.40 (s, 1H), 9.36 (s, 1H).

### Example 72

### 1-[(Isopropoxycarbonyl)oxy]ethyl 1-ethyl-5-(isoquinolin-4-ylamino)-6-oxo-3-phenyl-1,6-dihydropyridazine-4-carboxylate

Obtained as a solid (50%) from the title compound of Preparation 30 and the title compound from Preparation 46 following the procedure of Example 71.

LRMS (m/z): 517 (M+1)⁺.

Retention Time: 18 min.

δ(DMSO-d6): 1.01 (d, 3H), 1.12 (t, 6H), 1.37 (t, 3H), 4.21 (q, 2H), 4.55 (m, 1H), 5.30 (q, 1H), 7.24-7.40 (m, 5H), 7.72 (t, 1H), 7.82 (t, 1H), 7.98 (d, 1H), 8.16 (d, 1H), 8.28 (s, 1H), 9.19 (s, 1H), 9.38 (bs, 1H).

### Example 73

### 1-[(Isopropoxycarbonyl)oxy]ethyl 1-ethyl-5-[(4-methylpyridin-3-yl)amino]-6-oxo-3-phenyl-1,6-dihydropyridazine-4-carboxylate

Obtained as a solid (73%) from the title compound of Preparation 29 and the title compound from Preparation 46 following the procedure of Example 71.

LRMS (m/z): 481 (M+1)⁺.

Retention Time: 16 min.

δ(DMSO-d6): 0.87 (d, 3H), 1.18 (d, 3H), 1.19 (d, 3H), 2.21 (s, 3H), 1.35 (t, 3H), 4.18 (q, 2H), 4.65 (m, 1H), 5.60 (q, 1H), 7.24-7.40 (m, 6H), 8.22 (s, 1H), 8.29 (d, 1H), 9.03 (s, 1H).

### Example 74

### 1-{[(Cyclohexyloxy)carbonyl]oxy}ethyl 1-ethyl-6-oxo-3-phenyl-5-(pyridin-3-ylamino)-1,6-dihydropyridazine-4-carboxylate

Obtained as a solid (23%) from the title compound of Preparation 28 and the title compound of Preparation 47 following the procedure of Example 71.

LRMS (m/z): 507 (M+1)⁺.

Retention Time: 18 min.

δ(DMSO-d6): 0.85 (d, 3H), 1.34 (t, 3H), 1.20-1.50 (m, 6H), 1.62 (m, 2H), 1.76 (m, 2H), 4.18 (q, 2H), 4.45 (m, 1H), 5.80 (q, 1H), 7.28-7.40 (m, 6H), 7.49 (d, 1H), 8.31 (d, 1H), 8.40 (s, 1H), 9.35 (s, 1H).

### Example 75

### 1-{[(Cyclohexyloxy)carbonyl]oxy}ethyl 1-ethyl-5-(isoquinolin-4-ylamino)-6-oxo-3-phenyl-1,6-dihydropyridazine-4-carboxylate

Obtained as a solid (54%) from the title compound of Preparation 30 and the title compound from Preparation 47 following the procedure of Example 71.

LRMS (m/z): 557 (M+1)⁺.

Retention Time: 19 min.

δ(DMSO-d6): 0.41 (d, 3H), 1.20-1.45 (m, 6H), 1.37 (t, 3H), 1.57 (m, 2H), 1.68 (m, 2H), 4.21 (q, 2H), 4.32 (m, 1H), 5.29 (q, 1H), 7.24-7.35 (m, 5H), 7.72 (t, 1H), 7.82 (t, 1H), 7.98 (d, 1H), 8.16 (d, 1H), 8.28 (s, 1H), 9.19 (s, 1H), 9.38 (bs, 1H).

### Example 76

### 1-([(Cyclohexyloxy)carbonyl]oxy)ethyl 1-ethyl-5-[(4-methylpyridin-3-yl)amino]-6-oxo-3-phenyl-1,6-dihydropyridazine-4-carboxylate

Obtained as a solid (46%) from the title compound of Preparation 29 and the title compound from Preparation 47 following the procedure of Example 71.

LRMS (m/z): 521 (M+1)⁺.

Retention Time: 19 min.

δ(DMSO-d6): 0.89 (d, 3H), 1.20-1.50 (m, 6H), 1.35 (t, 3H), 1.62, (m, 2H), 1.77 (m, 2H), 2.22 (s, 3H), 4.18 (q, 2H), 4.43 (m, 1H), 5.57 (q, 1H), 7.24-7.40 (m, 6H), 8.23 (s, 1H), 8.28 (d, 1H), 9.07 (s, 1H).

### Example 77

### Ethyl 1-ethyl-6-oxo-3-phenyl-5-(thieno[2,3-c]pyridin-3-ylamino)-1,6-dihydropyridazine-4-carboxylate

Obtained as a solid (10%) from the title compound of Preparation 4 and 3-bromothieno[2,3-c]pyridine (S. Gronowitz, E. Sandberg. Arkiv for Kemi Band 32 nr21, 1970) following the experimental procedure of Example 15.

LRMS (m/z): 421 (M+1)⁺.

Retention Time: 13 min.

δ(CDCl₃): 0.50 (t, 3H), 1.47 (t, 3H), 3.15 (q, 2H), 4.34 (q, 2H), 7.35 (m, 4H), 7.43 (m, 1H), 7.68 (d, 1H), 7.76 (s, 1H), 8.57 (d, 1H), 9.12 (s, 1H).

### Example 78

### Ethyl i-ethyl-6-oxo-3-phenyl-5-(thieno[2,3-b]pyridin-3-ylamino)-1,6-dihydropyridazine-4-carboxylate

Obtained as a solid (16%) from the title compound of Preparation 4 and 3-bromothieno[2,3-b]pyridine (Klemm L. H., Merrill R. E., Lee F. H. W., Klopfenstein, C. E. Journal of Heterocuclic Chemistry 1974, 11(2), 205-209) following the experimental procedure of Example 15.

LRMS: m/Z 421 (M+1)⁺.

Retention Time: 9.2 min*. *Chromatographic method B.

δ(CDCl₃): 0.57 (t, 3H), 1.52 (t, 3H), 3.25 (q, 2H), 4.39 (q, 3H), 7.39 (m, 6H), 7.79 (s, 1H), 8.15 (d, 1H), 8.68 (s, 1H).

### Example 79

### [(2,2-Dimethylpropanoyl)oxy]methyl 1-ethyl-6-oxo-3-phenyl-5-(thieno[2,3-b]pyridin-3-ylamino)-1,6-dihydropyridazine-4-carboxylate

Obtained as a solid (10%) from the title compound of Preparation 49 following the procedure of Example 17.

LRMS: m/Z 507 (M+1)⁺.

Retention Time: 18 min.

δ(CDCl₃): 1.09 (s, 9H), 2.05 (t, 3H), 4.39 (q, 2H), 4.70 (s, 2H), 7.38 (m, 6H), 7.97 (s, 1H), 8.18 (d, 1H), 8.72 (s, 1H).

### Example 80-85

### 7-Ethoxy-7-oxoheptyl 1-ethyl-5-(isoquinolin-4-ylamino)-6-oxo-3-(3-thienyl)-1,6-dihydropyridazine-4-carboxylate

### 6-Ethoxy-6-oxohexyl 1-ethyl-5-(isoquinolin-4-ylamino)-6-oxo-3-(3-thienyl)-1,6-dihydropyridazine-4-carboxylate

### 3-Amino-3-oxopropyl 1-ethyl-5-(isoquinolin-4-ylamino)-6-oxo-3-(3-thienyl)-1,6-dihydropyridazine-4-carboxylate

### Benzyl 1-ethyl-5-(isoquinolin-4-ylamino)-6-oxo-3-(3-thienyl)-1,6-dihydropyridazine-4-carboxylate

### 4-(Methoxycarbonyl)benzyl 1-ethyl-5-(isoquinolin-4-ylamino)-6-oxo-3-(3-thienyl)-1,6-dihydropyridazine-4-carboxylate

### 4-Fluorobenzyl 1-ethyl-5-(isoquinolin-4-ylamino)-6-oxo-3-(3-thienyl)-1,6-dihydropyridazine-4-carboxylate

The title compounds were synthesized from the title compound of Preparation 37 and the corresponding bromide or chloride following the procedure of Example 59. The ESI/MS data and HPLC retention times are summarized in Table 2.

TABLE 2

| | ESI/MS | Retention |
|---|---|---|
| EXAMPLE | m/e | Time (min) |
| 80 | 549 | 18 |
| 81 | 534 | 17 |
| 82 | 464 | 11 |
| 83 | 483 | 17 |
| 84 | 541 | 17 |
| 85 | 501 | 14 |

### Example 86-95

### 2-Ethoxy-2-oxoethyl 1-ethyl-5-[(4-methylpyridin-3-yl)amino]-6-oxo-3-(2-thienyl)-1,6-dihydropyridazine-4-carboxylate

### 2-(Benzyloxy)-2-oxoethyl 1-ethyl-5-[(4-methylpyridin-3-yl)amino]-6-oxo-3-(2-thienyl)-1,6-dihydropyridazine-4-carboxylate

### Benzyl 1-ethyl-5-[(4-methylpyridin-3-yl)amino]-6-oxo-3-(2-thienyl)-1,6-dihydropyridazine-4-carboxylate

### 4-(Methoxycarbonyl)benzyl 1-ethyl-5-[(4-methylpyridin-3-yl)amino]-6-oxo-3-(2-thienyl)-1,6-dihydropyridazine-4-carboxylate

### 4-Fluorobenzyl 1-ethyl-5-[(4-methylpyridin-3-yl)amino]-6-oxo-3-(2-thienyl)-1,6-dihydropyridazine-4-carboxylate

### 6-Ethoxy-6-oxohexyl 1-ethyl-5-[(4-methylpyridin-3-yl)amino]-6-oxo-3-(2-thienyl)-1,6-dihydropyridazine-4-carboxylate

### 7-Ethoxy-7-oxoheptyl 1-ethyl-5-[(4-methylpyridin-3-yl)amino]-6-oxo-3-(2-thienyl)-1,6-dihydropyridazine-4-carboxylate

### (Acetyloxy)methyl 1-ethyl-5-[(4-methylpyridin-3-yl)amino]-6-oxo-3-(2-thienyl)-1,6-dihydropyridazine-4-carboxylate

### [(Isopropoxycarbonyl)oxy]methyl 1-ethyl-5-[(4-methylpyridin-3-yl)amino]-6-oxo-3-(2-thienyl)-1,6-dihydropyridazine-4-carboxylate

### {[(Cyclohexyloxy)carbonyl]oxy}methyl 1-ethyl-5-[(4-methylpyridin-3-yl)amino]-6-oxo-3-(2-thienyl)-1,6-dihydropyridazine-4-carboxylate

The title compounds were synthesized from the title compound of Preparation 35 and the corresponding bromide or chloride following the procedure of Example 50. The ESI/MS data and HPLC retention times are summarized in Table 3.

TABLE 3

| | ESI/MS | Retention |
|---|---|---|
| EXAMPLE | m/e | Time (min) |
| 86 | 443 | 15 |
| 87 | 505 | 17 |
| 88 | 447 | 17 |
| 89 | 505 | 17 |
| 90 | 465 | 17 |
| 91 | 499 | 17 |
| 92 | 513 | 17 |
| 93 | 429 | 14 |
| 94 | 473 | 16 |
| 95 | 513 | 18 |

### Example 96-102

### 4-Fluorobenzyl 1-ethyl-6-oxo-5-(pyridin-3-ylamino)-3-(2-thienyl)-1,6-dihydropyridazine-4-carboxylate

### 4-(Methoxycarbonyl)benzyl 1-ethyl-6-oxo-5-(pyridin-3-ylamino)-3-(2-thienyl)-1,6-dihydropyridazine-4-carboxylate

### 7-Ethoxy-7-oxoheptyl 1-ethyl-6-oxo-5-(pyridin-3-ylamino)-3-(2-thienyl)-1,6-dihydropyridazine-4-carboxylate

### 6-Ethoxy-6-oxohexyl 1-ethyl-6-oxo-5-(pyridin-3-ylamino)-3-(2-thienyl)-1,6-dihydropyridazine-4-carboxylate

### (Acetyloxy)methyl 1-ethyl-6-oxo-5-(pyridin-3-ylamino)-3-(2-thienyl)-1,6-dihydropyridazine-4-carboxylate

### [(Isopropoxycarbonyl)oxy]methyl 1-ethyl-6-oxo-5-(pyridin-3-ylamino)-3-(2-thienyl)-1,6-dihydropyridazine-4-carboxylate

### {[(Cyclohexyloxy)carbonyl]oxy}methyl 1-ethyl-6-oxo-5-(pyridin-3-ylamino)-3-(2-thienyl)-1,6-dihydropyridazine-4-carboxylate

The title compounds were synthesized from the title compound of Preparation 50 and the corresponding bromide or chloride following the procedure of Example 50. The ESI/MS data and HPLC retention times are summarized in Table 4.

TABLE 4

| | ESI/MS | Retention |
|---|---|---|
| EXAMPLE | m/e | Time (min) |
| 96 | 451 | 17 |
| 97 | 491 | 16 |
| 98 | 499 | 17 |
| 99 | 485 | 16 |
| 100 | 415 | 13 |
| 101 | 459 | 15 |
| 102 | 499 | 18 |

### Example 103-107

### 6-Ethoxy-6-oxohexyl 1-ethyl-5-(isoquinolin-4-ylamino)-6-oxo-3-(2-thienyl)-1,6-dihydropyridazine-4-carboxylate

### 7-Ethoxy-7-oxoheptyl 1-ethyl-5-(isoquinolin-4-ylamino)-6-oxo-3-(2-thienyl)-1,6-dihydropyridazine-4-carboxylate

### (Acetyloxy)methyl 1-ethyl-5-(isoquinolin-4-ylamino)-6-oxo-3-(2-thienyl)-1,6-dihydropyridazine-4-carboxylate

### [(Isopropoxycarbonyl)oxy]methyl 1-ethyl-5-(isoquinolin-4-ylamino)-6-oxo-3-(2-thienyl)-1,6-dihydropyridazine-4-carboxylate

### {[(Cyclohexyloxy)carbonyl]oxy}methyl 1-ethyl-5-(isoquinolin-4-ylamino)-6-oxo-3-(2-thienyl)-1,6-dihydropyridazine-4-carboxylate

The title compounds were synthesized from the title compound of Preparation 36 and the corresponding bromide or chloride following the procedure of Example 53. The ESI/MS data and HPLC retention times are summarized in Table 5.

TABLE 5

| | ESI/MS | Retention |
|---|---|---|
| EXAMPLE | m/e | Time (min) |
| 103 | 535 | 17 |
| 104 | 549 | 18 |
| 105 | 465 | 14 |
| 106 | 509 | 16 |
| 107 | 549 | 18 |

### Example 108

### Ethyl 1-ethyl-6-oxo-5-(pyridin-3-ylamino)-3-(3-thienyl)-1,6-dihydropyridazine-4-carboxylate

Obtained as a solid (39%) from the title product of Preparation 26 and 3-bromopyridine following the experimental procedure described in Example 43.

m.p.: 147.5-148.2° C.

LRMS: m/Z 471 (M+1)⁺.

Retention Time: 13 min.

### Example 109-118

### 2-(Acetyloxy)ethyl 1 ethyl-6-oxo-5-(pyridin-3-ylamino)-3-(3-thienyl)-1,6-dihydropyridazine-4-carboxylate

### 2-[(tert-Butoxycarbonyl)amino]ethyl 1 ethyl-6-oxo-5-(pyridin-3-ylamino)-3-(3-thienyl)-1,6-dihydropyridazine-4-carboxylate

### 2-Ethoxy-2-oxoethyl 1-ethyl-6-oxo-5-(pyridin-3-ylamino)-3-(3-thienyl)-1,6-dihydropyridazine-4-carboxylate

### 2-(Benzyloxy)-2-oxoethyl 1-ethyl-6-oxo-5-(pyridin-3-ylamino)-3-(3-thienyl)-1,6-dihydropyridazine-4-carboxylate

### Benzyl 1-ethyl-6-oxo-5-(pyridin-3-ylamino)-3-(3-thienyl)-1,6-dihydropyridazine-4-carboxylate

### 4-Fluorobenzyl 1-ethyl-6-oxo-5-(pyridin-3-ylamino)-3-(3-thienyl)-1,6-dihydropyridazine-4-carboxylate

### 4-(Methoxycarbonyl)benzyl 1-ethyl-6-oxo-5-(pyridin-3-ylamino)-3-(3-thienyl)-1,6-dihydropyridazine-4-carboxylate

### 7-Ethoxy-7-oxoheptyl 1-ethyl-6-oxo-5-(pyridin-3-ylamino)-3-(3-thienyl)-1,6-dihydropyridazine-4-carboxylate

### 6-Ethoxy-6-oxohexyl 1-ethyl-6-oxo-5-(pyridin-3-ylamino)-3-(3-thienyl)-1,6-dihydropyridazine-4-carboxylate

### {[(Cyclohexyloxy)carbonyl]oxy}methyl 1-ethyl-6-oxo-5-(pyridin-3-ylamino)-3-(3-thienyl)-1,6-dihydropyridazine-4-carboxylate

The title compounds were synthesized from the title compound of Preparation 50 and the corresponding bromide or chloride following the procedure of Example 50. The ESI/MS data and HPLC retention times are summarized in Table 6.

TABLE 6

| | ESI/MS | Retention |
|---|---|---|
| EXAMPLE | m/e | Time (min) |
| 109 | 429 | 13 |
| 110 | 486 | 15 |
| 111 | 429 | 14 |
| 112 | 491 | 16 |
| 113 | 433 | 16 |
| 114 | 451 | 16 |
| 115 | 491 | 16 |
| 116 | 499 | 17 |
| 117 | 485 | 16 |
| 118 | 499 | 17 |

### Example 119

### Ethyl 1-ethyl-5-[(4-methylpyridin-3-yl)amino]-6-oxo-3-(3-thienyl)-1,6-dihydropyridazine-4-carboxylate

Obtained as a solid (26%) from the title product of Preparation 26 and 3-bromo-4-methylpyridine following the experimental procedure described in Example 43.

m.p.: 182.6-183.4° C.

LRMS: m/Z 385 (M+M)⁺.

Retention Time: 14 min.

### Example 120-129

### 2-(Acetyloxy)ethyl 1-ethyl-5-[(4-methylpyridin-3-yl)amino]-6-oxo-3-(3-thienyl)-1,6-dihydropyridazine-4-carboxylate

### 2-[(tert-Butoxycarbonyl)amino]ethyl 1-ethyl-5-[(4-methylpyridin-3-yl)amino]-6-oxo-3-(3-thienyl)-1,6-dihydropyridazine-4-carboxylate

### 2-Ethoxy-2-oxoethyl 1-ethyl-5-[(4-methylpyridin-3-yl)amino]-6-oxo-3-(3-thienyl)-1,6-dihydropyridazine-4-carboxylate

### 2-(Benzyloxy)-2-oxoethyl 1-ethyl-5-[(4-methylpyridin-3-yl)amino]-6-oxo-3-(3-thienyl)-1,6-dihydropyridazine-4-carboxylate

### Benzyl 1-ethyl-5-[(4-methylpyridin-3-yl)amino]-6-oxo-3-(3-thienyl)-1,6-dihydropyridazine-4-carboxylate

### 3-Amino-3-oxopropyl 1-ethyl-5-[(4-methylpyridin-3-yl)amino]-6-oxo-3-(3-thienyl)-1,6-dihydropyridazine-4-carboxylate

### 4-Fluorobenzyl 1-ethyl-5-[(4-methylpyridin-3-yl)amino]-6-oxo-3-(3-thienyl)-1,6-dihydropyridazine-4-carboxylate

### 4-(Methoxycarbonyl)benzyl 1-ethyl-5-[(4-methylpyridin-3-yl)amino]-6-oxo-3-(3-thienyl)-1,6-dihydropyridazine-4-carboxylate

### 7-Ethoxy-7-oxoheptyl 1-ethyl-5-[(4-methylpyridin-3-yl)amino]-6-oxo-3-(3-thienyl)-1,6-dihydropyridazine-4-carboxylate

### 6-Ethoxy-oxohexyl 1-ethyl-5-[(4-methylpyridin-3-yl)amino]-6-oxo-3-(3-thienyl)-1,6-dihydropyridazine-4-carboxylate

The title compounds were synthesized from the title compound of Preparation 51 and the corresponding bromide or chloride following the procedure of Example 50. The ESI/MS data and HPLC retention times are summarized in Table 7.

TABLE 7

| | ESI/MS | Retention |
|---|---|---|
| EXAMPLE | m/e | Time (min) |
| 120 | 443 | 13 |
| 121 | 500 | 15 |
| 122 | 443 | 14 |
| 123 | 505 | 17 |
| 124 | 447 | 16 |
| 125 | 428 | 10 |
| 126 | 465 | 16 |
| 127 | 505 | 16 |
| 128 | 513 | 17 |
| 129 | 499 | 16 |

### Examples 130 and 131

### 1-{[(Cyclohexyloxy)carbonyl]oxy}ethyl 1-ethyl-5-[(4-methylpyridin-3-yl)amino]-6-oxo-3-phenyl-1,6-dihydropyridazine-4-carboxylate enantiomer 1

### 1-{[(Cyclohexyloxy)carbonyl]oxy}ethyl 1-ethyl-5-[(4-methylpyridin-3-yl)amino]-6-oxo-3-phenyl-1,6-dihydropyridazine-4-carboxylate enantiomer 2

A solution of the title product of Example 76 (1.28 g) in methanol (32 mL) was injected (32×1 mL) onto a Chiralpak AD-H semi-preparative (250×20 mm, 5 μm) HPLC column, eluting with acetonitrile (containing a 0.1% of formic acid)/water, 9:1, at 17 mL/min with UV detection at 300 nm. The enantiomers were separated with the faster eluting enantiomer having a retention time of 4.8 min (enantiomer 1, example 130) and the slower eluting enantiomer having a retention time of 6.6 min (enantiomer 2, example 131). The eluants were concentrated to provide the enantiomers as white solids: Enantiomer 1 (335 mg), Enantiomer 2 (304 mg).

### Example 130

### Enantiomer 1

LRMS: m/Z 521 (M+1)⁺.

Retention Time*: 4.0 min. *Chromatographic analysis using a Chiralpak AD-H (250×4.6 mm) analytical HPLC column eluting with ace-tonitile (containing a 0.1% AcOH)/water, 9:1 at 1 mL/min

ee: 100%

### Example 131

### Enantiomer 2

LRMS: m/Z 521 (M+1)⁺.

Retention Time*¹: 5.4 min. ¹chromatographic analysis using a Chiralpak AD-H (250×4.6 mm) analytical HPLC column eluting with ace-tonitrile (containing a 0.1% AcOH)/water, 9:1 at 1 mL/min.

ee: 99.5%

### Examples 132 and 133

### (−)-1-{[(Cyclohexyloxy)carbonyl]oxy}ethyl 1-ethyl-6-oxo-3-phenyl-5-(pyridin-3-ylamino)-1,6-dihydropyridazine-4-carboxylate

### (+)-{[(Cyclohexyloxy)carbonyl]oxy}ethyl 1-ethyl-6-oxo-3-phenyl-5-(pyridin-3-ylamino)-1,6-dihydropyridazine-4-carboxylate

A solution of the title product of Example 74 (2.00 g) in methanol (20 mL) was injected (20×1 mL) onto a Chiralpak AD-H semi-preparative (250×20 mm, 5 μm) HPLC column, eluting with acetonitrile (containing a 0.1% of formic acid)/water, 9:1, at 17 mL/min with UV detection at 300 nm. The enantiomers were separated with the faster eluting enantiomer having a retention time of 5.5 min (enantiomer 1, example 132) and the slower eluting enantiomer having a retention time of 8.0 min (enantiomer 2, example 133). The eluents were concentrates to provide the enantiomers as white solids: Enantiomer 1 (808 mg), Enantiomer 2 (767 mg).

### Example 132

### Enantiomer 1

LRMS: m/Z 507 (M+1)⁺.

Retention Time*²: 9.7 min. ²Chromatographic analysis using a Chiralpak AD-H (250×4.6 mm) analytical HPLC column eluting with hex-ane/etanol 6:4 at 1 mL/min.

ee: 98.1%

[α]_{D}=−52.6 (c 1.0, AcCN)

### Example 133

### Enantiomer 2

LRMS: m/Z 507 (M+1)⁺.

Retention Time*²: 15.1 min.

ee: 99.3%

[α]_{D}=+57.9 (c 1.0, AcCN)

### Example 134

### 1-{[(1-Ethylpropoxy)carbonyl]oxy}ethyl 1-ethyl-5-[(4-methylpyridin-3-yl)amino]-6-oxo-3-phenyl-1,6-dihydropyridazine-4-carboxylate

Obtained as a solid (46%) from the title compound of Preparation 29 and carbonic acid 1-chloro-ethyl ester 1-ethyl-propyl ester following the procedure of Example 71.

m.p.: 95.8-96.1° C.

δ(DMSO-d₆): 0.74 (t, 3H), 0.81 (t, 3H), 0.91 (d, 3H), 1.35 (t, 3H), 1.53 (m, 4H), 2.22 (s, 3H), 4.18 (m, 2H), 4.38 (m, 1H), 5.52 (q, 1H), 7.26-7.37 (m, 6H), 8.23 (s, 1H), 8.28 (s, 1H), 9.03 (s, 1H).

### Example 135

### (Acetyloxy)methyl 1-ethyl-5-[(4-methylpyridin-3-yl)amino]-6-oxo-3-phenyl-1,6-dihydropyridazine-4-carboxylate

Obtained as a solid (71%) from the title compound of Preparation 29 and acetic acid bromomethyl ester following the procedure of Example 71.

LRMS: m/Z 423 (M+1)⁺.

Retention Time: 14 min.

### Example 136

### {[(Cyclohexyloxy)carbonyl]oxy}methyl 1-ethyl-5-[(4-methylpyridin-3-yl)amino]-6-oxo-3-phenyl-1,6-dihydropyridazine-4-carboxylate

Obtained as a solid (21%) from the title compound of Preparation 29 and carbonic acid chloromethyl ester cyclohexyl ester following the procedure of Example 71.

m.p.: 113.9-114.8° C.

δ(DMSO-d6): 1.20-1.40 (m, 9H), 1.62 (m, 2H), 1.80 (m, 2H), 2.22 (s, 3H), 4.18 (q, 2H), 4.48 (m, 1H), 4.71 (s, 2H), 7.25-7.36 (m, 6H), 8.22 (s, 1H), 8.29 (d, 1H), 9.07 (s, 1H).

### Example 137

### (Isobutyryloxy)methyl 1-ethyl-5-[(4-methylpyridin-3-yl)amino]-6-oxo-3-phenyl-1,6-dihydropyridazine-4-carboxylate

Obtained as a solid (38%) from the title compound of Preparation 29 and isobutyric acid chloromethyl ester following the procedure of Example 71.

m.p.: 162.3-163.1° C.

δ(DMSO-d₆): 0.95 (d, 6H), 1.33 (t, 2H), 2.21 (s, 3H), 2.32 (quint, 1H), 4.16 (q, 2H), 4.68 (s, 2H), 7.25-7.36 (m, 6H), 8.21 (s, 1H), 8.30 (d, 1H), 9.05 (s, 1H).

### Example 138

### [(Isopropoxycarbonyl)oxy]methyl 1-ethyl-5-[(4-methylpyridin-3-yl)amino]-6-oxo-3-phenyl-1,6-dihydropyridazine-4-carboxylate

Obtained as a solid (20%) from the title compound of Preparation 29 and carbonic acid chloromethyl ester isopropyl ester following the procedure of Example 71.

m.p.: 145.6-147.0° C.

δ(DMSO-d6): 1.21 (d, 6H), 1.33 (t, 2H), 2.22 (s, 3H), 4.18 (q, 2H), 4.70 (m, 3H), 7.25-7.37 (m, 6H), 8.22 (s, 1H), 8.30 (d, 1H), 9.08 (s, 1H).

### Example 139

### {[(1-Ethylpropoxy)carbonyl]oxy}methyl 1-ethyl-5-[(4-methylpyridin-3-yl)amino]-6-oxo-3-phenyl-1,6-dihydropyridazine-4-carboxylate

Obtained as a solid (9%) from the title compound of Preparation 29 and carbonic acid chloromethyl ester 1-ethyl-propyl ester following the procedure of Example 71.

m.p.: 123.7-124.3° C.

δ(DMSO-d₆): 0.81 (t, 6H), 1.33 (t, 3H), 1.55 (m, 4H), 2.21 (s, 3H), 4.18 (q, 2H), 4.46 (m, 1H), 4.72 (s, 1H), 7.26-7.37 (m, 6H), 8.23 (s, 1H), 8.32 (d, 1H), 9.09 (s, 1H).

### Example 140

### (Butyryloxy)methyl 1-ethyl-5-[(4-methylpyridin-3-yl)amino]-6-oxo-3-phenyl-1,6-dihydropyridazine-4-carboxylate

Obtained as a solid (10%) from the title compound of Preparation 29 and butyric acid chloromethyl ester following the procedure of Example 71.

m.p.: 117.1-117.9° C.

LRMS: m/Z 451 (M+1)⁺.

Retention Time: 16 min.

### Example 141

### (Propionyloxy)methyl 1 ethyl-5-[(4-methylpyridin-3-yl)amino]-6-oxo-3-phenyl-1,6-dihydropyridazine-4-carboxylate

Obtained as a solid (44%) from the title compound of Preparation 29 and propionic acid chloromethyl ester following the procedure of Example 71.

m.p.: 163.1-164.2° C.

δ(DMSO-d₆): 0.92 (t, 3H), 1.34 (t, 3H), 2.21 (q, 2H), 2.22 (s, 3H), 4.18 (q, 2H), 4.71 (s, 2H), 7.26-7.37 (m, 6H), 8.22 (s, 1H), 8.30 (d, 1H), 9.05 (s, 1H).

### Example 142

### (5-Methyl-2-oxo-1,3-dioxol-4-yl)methyl 1-ethyl-5-[(4-methylpyridin-3-yl)amino]-6-oxo-3-phenyl-1,6-dihydropyridazine-4-carboxylate

Obtained as a solid (82%) from the title compound of Preparation 29 and 4-chloromethyl-5-methyl-[1,3]dioxol-2-one following the procedure of Example 71.

m.p.: 197.3-198.1° C.

LRMS: m/Z 463 (M+1)⁺.

Retention Time: 14 min.

### Example 143

### 1-{[(1-Ethylpropoxy)carbonyl]oxy}ethyl 1-ethyl-5-(isoquinolin-4-ylamino)-6-oxo-3-phenyl-1,6-dihydropyridazine-4-carboxylate

Obtained as a solid (27%) from the title product of Preparation 30 and carbonic acid 1-chloro-ethyl ester 1-ethyl-propyl ester following the experimental procedure of Example 17.

m.p.: 126.8-127.3° C.

δ(DMSO-d₈): 0.41 (d, 3H), 0.67 (t, 3H), 0.75 (t, 3H), 1.38 (t, 3H), 1.47 (m, 4H), 4.25 (m, 2H), 4.30 (m, 1H), 5.22 (q, 1H), 7.32 (m, 5H), 7.74 (t, 1H), 7.82 (t, 1H), 8.00 (d, 1H), 8.18 (d, 1H), 8.29 (s, 1H), 9.20 (s, 1H), 9.39 (s, 1H).

### Example 144

### (Butyryloxy)methyl 1-ethyl-5-(isoquinolin-4-ylamino)-6-oxo-3-phenyl-1,6-dihydropyridazine-4-carboxylate

Obtained as a solid (38%) from the title product of Preparation 30 and butyric acid chloromethyl ester following the experimental procedure of Example 17.

LRMS: m/Z 487 (M+1)⁺.

Retention Time: 17 min.

δ(CDCl₃): 0.85 (t, 3H), 1.44 (m, 5H), 1.99 (t, 3H), 4.38 (q, 2H), 4.62 (s, 1H), 7.33 (m, 6H), 7.71 (t, 1H), 7.82 (t, 1H), 8.06 (m, 2H), 8.39 (s, 1H), 9.18 (s, 1H).

### Example 145

### (Isobutyryloxy)methyl 1 ethyl-5-(isoquinolin-4-ylamino)-6-oxo-3-phenyl-1,6-dihydropyridazine-4-carboxylate

Obtained as a solid (36%) from the title product of Preparation 30 and isobutyric acid chloromethyl ester following the experimental procedure of Example 17. Polimer-supported diisopropylethylamine was used as a base instead of diisopropylethylamine.

LRMS: m/Z 487 (M+1)⁺.

Retention Time: 17 min.

δ(CDCl₃): 0.97 (d, 6H), 1.49 (t, 3H), 2.24 (m, 1H), 4.36 (q, 2H), 4.59 (s, 2H), 7.33 (m, 6H), 7.73 (t, 1H), 7.83 (t, 1H), 8.07 (m, 2H), 8.40 (s, 1H), 9.19 (s, 1H).

### Example 146

### (5-Methyl-2-oxo-1,3-dioxol-4-yl)methyl 1-ethyl-5-(isoquinolin-4-ylamino)-6-oxo-3-phenyl-1,6-dihydropyridazine-4-carboxylate

Obtained as a solid (59%) from the title product of Preparation 30 and 4-chloromethyl-5-methyl-[1,3]dioxol-2-one following the experimental procedure of Example 17.

m.p.: 205.5-203.2° C.

δ(DMSO-d₆): 1.38 (t, 3H), 1.70 (s, 3H), 3.69 (s, 2H), 4.22 (q, 2H), 7.19 (m, 2H), 7.28 (m, 3H), 7.71 (t, 1H), 7.83 (t, 1H), 7.97 (d, 1H), 8.17 (d, 1H), 8.29 (s, 1H), 9.21 (s, 1H), 9.41 (s, 1H).

### Example 147

### {[(Cyclohexyloxy)carbonyl]oxy}methyl 1-ethyl-5-(isoquinolin-4-ylamino)-6-oxo-3-phenyl-1,6-dihydropyridazine-4-carboxylate

Obtained as a solid (59%) from the title product of Preparation 30 and carbonic acid chloromethyl ester cyclohexyl ester following the experimental procedure of Example 17. Polimer-supported diisopropylethylamine was used as a base.

m.p.: 133.0-133.4° C.

δ(DMSO-d₆): 1.20-1.40 (m, 9H), 1.62 (m, 2H), 1.75 (m, 2H), 4.22 (m, 4H), 4.37 (m, 1H), 7.20-7.35 (m, 5H), 7.40 (t, 1H), 7.83 (t, 1H), 7.97 (d, 1H), 8.17 (d, 1H), 8.30 (s, 1H), 9.22 (s, 1H), 9.45 (s, 1H).

### Example 148

### [(Isopropoxycarbonyl)oxy]methyl 1-ethyl-5-(isoquinolin-4-ylamino)-6-oxo-3-phenyl-1,6-dihydropyridazine-4-carboxylate

Obtained as a solid (94%) from the title product of Preparation 30 and carbonic acid chloromethyl ester isopropyl ester following the experimental procedure of Example 17. Polimer-supported diisopropylethylamine was used as a base.

m.p.: 132.0-133.0° C.

δ(DMSO-d₆): 1.17 (d, 6H), 1.38 (t, 3H), 4.25 (m, 4H), 4.61 (m, 1H), 7.21-7.36 (m, 5H), 7.74 (t, 1H), 7.84 (t, 1H), 7.98 (d, 1H), 8.18 (d, 1H), 8.31 (s, 1H), 9.22 (s, 1H), 9.46 (s, 1H).

### Example 149

### {[(1-Ethylpropoxy)carbonyl]oxy}methyl 1-ethyl-5-(isoquinolin-4-ylamino)-6-oxo-3-phenyl-1,6-dihydropyridazine-4-carboxylate

Obtained as a solid (22%) from the title product of Preparation 30 and carbonic acid chloromethyl ester 1-ethyl-propyl ester following the experimental procedure of Example 17. Polimer-supported diisopropylethylamine was used as a base.

m.p.: 109.6-110.7° C.

δ(DMSO-d₆): 0.77 (t, 6H), 1.38 (t, 3H), 1.48 (m, 4H), 4.22 (m, 4H), 4.35 (m, 1H), 7.21-7.33 (m, 5H), 7.72 (t, 1H), 7.84 (t, 1H), 7.98 (d, 1H), 8.17 (d, 1H), 8.30 (s, 1H), 9.22 (s, 1H), 9.45 (s, 1H).

### Example 150

### 2-Methoxy-2-oxoethyl 1-ethyl-5-(isoquinolin-4-ylamino)-6-oxo-3-phenyl-1,6-dihydropyridazine-4-carboxylate

Obtained as a solid (10%) from the title product of Preparation 30 and chloro-acetic acid methyl ester following the experimental procedure of Example 17. Polimer-supported diisopropylethylamine was used as a base.

m.p.: 175.0-176.2° C.

δ(DMSO-d₆): 1.40 (t, 3H), 3.05 (s, 2H), 3.42 (s, 3H), 4.23 (m, 2H), 7.33 (m, 5H), 7.74 (t, 1H), 7.81 (t, 1H), 7.98 (d, 1H), 8.18 (d, 1H), 8.27 (s, 1H), 9.22 (s, 1H), 9.41 (s, 1H).

### Example 151

### (Acetyloxy)methyl 1-ethyl-5-(isoquinolin-4-ylamino)-6-oxo-3-phenyl-1,6-dihydropyridazine-4-carboxylate

Obtained as a solid (13%) from the title product of Preparation 30 and acetic acid bromomethyl ester following the experimental procedure of Example 17. Polimer-supported diisopropylethylamine was used as a base instead of potassium carbonate.

m.p.: 133.5-134.4° C.

δ(DMSO-d₆): 1.38 (t, 3H), 1.76 (s, 3H), 3.05 (s, 2H), 4.25 (m, 4H), 7.23 (m, 2H), 7.33 (m, 3H), 7.75 (t, 1H), 7.83 (t, 1H), 7.98 (d, 1H), 8.18 (d, 1H), 8.30 (s, 1H), 9.24 (s, 1H), 9.44 (s, 1H).

### Example 152

### (Propionyloxy)methyl 1-ethyl-5-(isoquinolin-4-ylamino)-6-oxo-3-phenyl-1,6-dihydropyridazine-4-carboxylate

Obtained as a solid (54%) from the title product of Preparation 30 and propionic acid chloromethyl ester following the experimental procedure of Example 17. Polimer-supported diisopropylethylamine was used as a base.

m.p.: 122.7-123.4° C.

δ(DMSO-d₈): 0.85 (t, 3H), 1.37 (t, 3H), 2.02 (q, 2H), 4.25 (m, 4H), 7.22 (m, 2H), 7.33 (m, 3H), 7.74 (t, 1H), 7.84 (t, 1H), 7.98 (d, 1H), 8.18 (d, 1H), 8.30 (s, 1H), 9.23 (s, 1H), 9.44 (s, 1H).

### Example 153

### (Isobutyryloxy)methyl 1-ethyl-6-oxo-3-phenyl-5-(pyridin-3-ylamino)-1,6-dihydropyridazine-4-carboxylate

Obtained as a solid (8%) from the title product of Preparation 28 and isobutyric acid chloromethyl ester following the experimental procedure of Example 17.

LRMS: m/Z 437 (M+1)⁺.

Retention Time: 16 min

### Example 154

### [(Isopropoxycarbonyl)oxy]methyl 1-ethyl-6-oxo-3-phenyl-5-(pyridin-3-ylamino)-1,6-dihydropyridazine-4-carboxylate

Obtained as a solid (16%) from the title product of Preparation 28 and carbonic acid chloromethyl ester isopropyl ester following the experimental procedure of Example 17.

LRMS: m/Z 453 (M+1)⁺.

Retention Time: 16 min

δ(DMSO-d6): 1.22 (d, 6H), 1.34 (t, 3H), 4.18 (q, 2H), 4.70 (m, 1H), 4.86 (s, 2H), 7.35 (m, 7H), 8.37 (m, 2H), 9.37 (s, 1H).

### Example 155

### [(2,2-Dimethylbutanoyl)oxy]methyl 1-ethyl-6-oxo-3-phenyl-5-(pyridin-3-ylamino)-1,6-dihydropyridazine-4-carboxylate

Obtained as a solid (27%) from the title product of Preparation 28 and 2,2-dimethyl-butyric acid chloromethyl ester following the experimental procedure of Example 17.

LRMS: m/Z 493 (M+1)⁺.

Retention Time: 17 min

δ(DMSO-d6): 0.67 (t, 3H), 0.98 (s, 6H), 1.34 (m, 5H), 4.18 (m, 2H), 4.81 (s, 2H), 7.32 (m, 6H), 7.45 (m, 2H), 8.39 (s, 1H), 9.38 (s, 1H)

### Example 156

### {[(Cyclohexyloxy)carbonyl]oxy}methyl 1-ethyl-6-oxo-3-phenyl-5-(pyridin-3-ylamino)-1,6-dihydropyridazine-4-carboxylate

Obtained as a solid (9%) from the title product of Preparation 28 and carbonic acid chloromethyl ester cyclohexyl ester following the experimental procedure of Example 17.

LRMS: m/Z 493 (M+1)⁺.

Retention Time: 17 min

δ(DMSO-d6): 1.34 (m, 9H), 1.64 (m, 2H), 1.80 (m, 2H), 4.18 (q, 2H), 4.48 (m, 1H), 4.86 (s, 2H), 7.32 (m, 6H), 7.50 (m, 1H), 8.36 (m, 2H), 9.37 (s, 1H).

### Example 157

### {[(1-Ethylpropoxy)carbonyl]oxy}methyl 1-ethyl-6-oxo-3-phenyl-5-(pyridin-3-ylamino)-1,6-dihydropyridazine-4-carboxylate

Obtained as a solid (33%) from the title product of Preparation 28 and carbonic acid chloromethyl ester 1-ethyl-propyl ester following the experimental procedure of Example 17.

LRMS: m/Z 481 (M+1)⁺.

Retention Time: 17 min

δ(DMSO-d6): 0.82 (t, 6H), 1.34 (t, 3H), 1.55 (m, 4H), 4.18 (q, 2H), 4.45 (m, 1H), 4.85 (s, 2H), 7.34 (m, 6H), 7.51 (m, 1H), 8.37 (m, 2H), 9.38 (s, 1H).

### Example 158

### [(2-Methylbutanoyl)oxy]methyl 1 ethyl-6-oxo-3-phenyl-5-(pyridin-3-ylamino)-1,6-dihydropyridazine-4-carboxylate

Obtained as a solid (47%) from the title product of Preparation 28 and 2-methyl-butyric acid chloromethyl ester following the experimental procedure of Example 17.

LRMS: m/Z 451 (M+1)⁺.

Retention Time: 17 min

### Example 159

### [(Dibutoxyphosphoryl)oxy]methyl 1-ethyl-6-oxo-3-phenyl-5-(pyridin-3-ylamino)-1,6-dihydropyridazine-4-carboxylate

Obtained as a solid (43%) from the title product of Preparation 28 and phosphoric acid dibutyl ester chloromethyl ester following the experimental procedure of Example 17.

LRMS: m/Z 559 (M+1)⁺.

Retention Time: 18 min

δ(DMSO-d6): 0.82 (t, 6H), 1.26 (m, 4H), 1.33 (t, 3H), 1.50 (m, 4H), 3.85 (m, 4H), 4.17 (q, 2H), 4.68 (d, 2H), 7.34 (m, 6H), 7.48 (m, 1H), 8.35 (m, 2H), 9.40 (s, 1H).

### Example 160

### {[(1-Ethylpropoxy)carbonyl]oxy}methyl 1-ethyl-3-(4-fluorophenyl)-6-oxo-5-(pyridin-3-ylamino)-1,6-dihydropyridazine-4-carboxylate

Obtained as a solid (44%) from the title product of Preparation 33 and carbonic acid chloromethyl ester 1-ethyl-propyl ester following the experimental procedure of Example 17.

LRMS: m/Z 499 (M+1)⁺.

Retention Time: 18 min

δ(DMSO-d6): 0.82 (t, 6H), 1.34 (t, 3H), 1.54 (m, 4H), 4.18 (q, 2H), 4.43 (m, 1H), 4.89 (s, 2H), 7.15 (m, 2H), 7.34 (m, 3H), 7.50 (m, 1H), 8.36 (m, 2H), 9.40 (s, 1H).

### Example 161

### {[(Cyclohexyloxy)carbonyl]oxy}methyl 1-ethyl-3-(4-fluorophenyl)-6-oxo-5-(pyridin-3-ylamino)-1,6-dihydropyridazine-4-carboxylate

Obtained as a solid (50%) from the title product of Preparation 28 and carbonic acid chloromethyl ester cyclohexyl ester following the experimental procedure of Example 17.

LRMS: m/Z 511 (M+1)⁺.

Retention Time: 18 min

δ(DMSO-d6): 1.34 (M, 7H), 1.48 (m, 2H), 1.64 (m, 2H), 1.80 (m, 2H), 4.17 (q, 2H), 4.45 (m, 1H), 4.90 (s, 2H), 7.16 (M, 2H), 7.33 (m, 3H), 7.51 (m, 1H), 8.35 (m, 2H), 9.40 (s, 1H).

### Example 162

Chloromethyl 1-ethyl-6-oxo-3-phenyl-5-(pyridin-3-ylamino)-1,6-dihydropyridazine-4-carboxylate

To a solution of the title compound of Preparation 28 (672 mg, 2 mmol), tetrabutyl-ammonium hydrogen sulfate (68 mg, 0.2 mmol) and sodium hydrogencarbonate (672 mg, 8 mmol) in 15 ml of water and 15 ml of dichloromethane at 0° C., chlorosulfuric acid chloromethyl ester (247 μl, 2.4 mmol) was added. The mixture was stirred at 0° C. for 30 min and at room temperature for 5 h. The organic layer was separated, washed with water, brine and dried over anhydrous sodium sulphate. Removal of the solvent under reduced pressure afforded 650 mg of a brown solid which was purified by column cromatography (CH₂Cl₂) to give the title compound as a yellow solid (520 mg, 68% yield).

LRMS (m/z): 385 (M+1)⁺.

Retention Time: 14 min.

δ(DMSO-d₆): 1.35 (t, 3H), 4.18 (q, 2H), 5.01 (s, 2H), 7.28-7.41 (m, 5H), 7.50 (m, 2H), 8.36 (m, 2H), 9.44 (s, 1H).

### Example 163

### Chloromethyl 1-ethyl-5-[(4-methylpyridin-3-yl)amino]-6-oxo-3-phenyl-1,6-dihydropyridazine-4-carboxylate

Obtained as a solid (56%) from the title compound of Preparation 29 following the procedure of Example 162.

LRMS (m/z): 399 (M+1)⁺.

Retention Time: 15 min.

δ(DMSO-d₆): 1.35 (t, 3H), 2.22 (s, 3H), 4.18 (q, 2H), 4.85 (s, 2H), 7.28-7.38 (m, 6H), 8.21 (s, 1H), 8.30 (d, 1H), 9.12 (s, 1H).

### Example 164

### Chloromethyl 1-ethyl-5-(isoquinolin-4-ylamino)-6-oxo-3-phenyl-1,6-dihydropyridazine-4-carboxylate

Obtained as a solid (64%) from the title compound of Preparation 30 following the procedure of Example 162.

LRMS (m/z): 435 (M+1)⁺.

Retention Time: 16 min.

δ(DMSO-d₆): 1.39 (t, 3H), 4.23 (q, 2H), 4.42 (s, 2H), 7.23-7.35 (m, 5H), 7.74 (ddd, 1H), 7.84 (ddd, 1H), 7.96 (dd, 1H), 8.17 (dd, 1H), 8.30 (d, 1H), 9.22 (d, 1H), 9.50 (s, 1H).

### Example 165

### Iodomethyl 1-ethyl-6-oxo-3-phenyl-5-(pyridin-3-ylamino)-1,6-dihydropyridazine-4-carboxylate

A solution of the title compound of Example 162 (200 mg, 0.52 mmol) and sodium iodide (130 mg, 0.86 mmol) in 8 ml of acetone was stirred at room temperature for 20 h. The solvent was removed under reduced pressure and dichloromethane was added. The organic layer was washed with Na₂S₂O₃, water, brine and dried over anhydrous sodium sulphate. Removal of the solvent under reduced pressure afforded 100 mg of a yellow product (30%).

LRMS (m/z): 477 (M+1)⁺.

Retention Time: 16 min.

### Example 166

### Fluoromethyl 1-ethyl-6-oxo-3-phenyl-5-(pyridin-3-ylamino)-1,6-dihydropyridazine-4-carboxylate

To a solution of the title compound of Example 14 (200 mg, 0.52 mmol) in 4 ml of acetone, silver (I) fluoride (98 mg, 0.78) was added portion wise. The mixture was stirred at room temperature for 20 h. The mixture was diluted with 30 ml of ethyl acetate and filtered through Zelite®. The solvent was removed under reduced pressure. Purification by reverse phase column chromatography (Biotage® 25M C18 preparative chromatography column H₂O:AcCN gradient from 0% AcCN to 100% AcCN) gave the title compound as a solid (18 mg, 11% yield).

LRMS (m/z): 369 (M+1)⁺.

Retention Time: 13 min.

### Example 167-176

### 1-{[(1-Ethylpropoxy)carbonyl]oxy}ethyl 1-ethyl-6-oxo-3-phenyl-5-(pyridin-3-ylamino)-1,6-dihydropyridazine-4-carboxylate

### (5-Methyl-2-oxo-1,3-dioxol-4-yl)methyl 1-ethyl-6-oxo-3-phenyl-5-(pyridin-3-ylamino)-1,6-dihydropyridazine-4-carboxylate

### ({N—[(Benzyloxy)carbonyl]-L-valyl}oxy)methyl 1-ethyl-6-oxo-3-phenyl-5-(pyridin-3-ylamino)-1,6-dihydropyridazine-4-carboxylate

### ({[1-Ethyl-6-oxo-3-phenyl-5-(pyridin-3-ylamino)-1,6-dihydropyridazin-4-yl]carbonyl}oxy)methyl N-(tert-butoxycarbonyl)-L-leucinate

({[1-Ethyl-6-oxo-3-phenyl-5-(pyridin-3-ylamino)-1,6-dihydropyridazin-4-yl]carbonyl}oxy)methyl morpholine-4-carboxylate

### {[(Methylamino)carbonyl]oxy}methyl 1-ethyl-6-oxo-3-phenyl-5-(pyridin-3-ylamino)-1,6-dihydropyridazine-4-carboxylate

### {[(Dimethylamino)carbonyl]oxy}methyl 1-ethyl-6-oxo-3-phenyl-5-(pyridin-3-ylamino)-1,6-dihydropyridazine-4-carboxylate

### (Propionyloxy)methyl 1 ethyl-6-oxo-3-phenyl-5-(pyridin-3-ylamino)-1,6-dihydropyridazine-4-carboxylate

### (Pentanoyloxy)methyl 1-ethyl-6-oxo-3-phenyl-5-(pyridin-3-ylamino)-1,6-dihydropyridazine-4-carboxylate

### 2-Oxo-1,3-dioxolan-4-yl 1-ethyl-6-oxo-3-phenyl-5-(pyridin-3-ylamino)-1,6-dihydropyridazine-4-carboxylate

The title compounds were synthesized from the title compound of Preparation 28 and the corresponding bromide or chloride following the procedure of Example 17. The ESI/MS data and HPLC retention times are summarized in Table 8.

TABLE 8

| | ESI/MS | Retention |
|---|---|---|
| EXAMPLE | m/e | Time (min) |
| 167 | 495 | 18 |
| 168 | 449 | 13 |
| 169 | 600 | 17 |
| 170 | 580 | 18 |
| 171 | 480 | 12 |
| 172 | 424 | 11 |
| 173 | 438 | 13 |
| 174 | 423 | 14 |
| 175 | 451 | 16 |
| 176 | 422 | 12 |

### Example 177

### Ethyl 1-ethyl-5-[(1,7-naphthyridin-5-ylamino)]-6-oxo-3-(3-thienyl)-1,6-dihydropyridazine-4-carboxylate

Obtained as a solid (48%) from the title product of Preparation 4 and 5-bromo-[1,7]naphthyridine following the experimental procedure described in Example 43.

LRMS: m/Z 416 (M+1)⁺.

Retention Time*: 9.2 min. *Chromatographic method B.

### Example 178

### 1-{[(Cyclohexyloxy)carbonyl]oxy}ethyl 1-ethyl-5-(1,7-naphthyridin-5-ylamino)-6-oxo-3-phenyl-1,6-dihydropyridazine-4-carboxylate

Obtained as a solid (20%) from the title product of Preparation 52 and carbonic acid cyclohexyl ester 1-chloro-ethyl ester following the experimental procedure of Example 17.

m.p.: 111.8-113.7° C.

δ(DMSO-d6): 0.51 (d, 3H), 1.25-1.39 (m, 9H), 1.62 (m, 2H), 1.75 (m, 2H), 4.18 (m, 2H), 4.32 (m, 1H), 5.42 (m, 1H), 7.26 (m, 2H), 7.34 (m, 3H), 7.81 (m, 1H), 8.41 (s, 1H), 8.43 (d, 1H), 9.08 (m, 1H), 9.26 (1H), 9.49 (s, 1H).

The following examples illustrate pharmaceutical compositions according to the present invention.

### COMPOSITION EXAMPLES

### Composition Example 1

Preparation of tablets
Formulation:

| | |
|---|---|
| Compound of the present invention | 5.0 mg |
| Lactose | 113.6 mg |
| Microcrystalline cellulose | 28.4 mg |
| Light silicic anhydride | 1.5 mg |
| Magnesium stearate | 1.5 mg |

Using a mixer machine, 15 g of the compound of the present invention are mixed with 340.8 g of lactose and 85.2 g of microcrystalline cellulose. The mixture is subjected to compression moulding using a roller compactor to give a flake-like compressed material. The flake-like compressed material is pulverised using a hammer mill, and the pulverised material is screened through a 20 mesh screen. A 4.5 g portion of light silicic anhydride and 4.5 g of magnesium stearate are added to the screened material and mixed. The mixed product is subjected to a tablet making machine equipped with a die/punch system of 7.5 mm in diameter, thereby obtaining 3,000 tablets each having 150 mg in weight.

### Composition Example 2

Preparation of coated tablets
Formulation:

| | |
|---|---|
| Compound of the present invention | 5.0 mg |
| Lactose | 95.2 mg |
| Corn starch | 40.8 mg |
| Polyvinylpyrrolidone K25 | 7.5 mg |
| Magnesium stearate | 1.5 mg |
| Hydroxypropylcellulose | 2.3 mg |
| Polyethylene glycol 6000 | 0.4 mg |
| Titanium dioxide | 1.1 mg |
| Purified talc | 0.7 mg |

Using a fluidised bed granulating machine, 15 g of the compound of the present invention are mixed with 285.6 g of lactose and 122.4 g of corn starch. Separately, 22.5 g of polyvinylpyrrolidone is dissolved in 127.5 g of water to prepare a binding solution. Using a fluidised bed granulating machine, the binding solution is sprayed on the above mixture to give granulates. A 4.5 g portion of magnesium stearate is added to the obtained granulates and mixed. The obtained mixture is subjected to a tablet making machine equipped with a die/punch biconcave system of 6.5 mm in diameter, thereby obtaining 3,000 tablets, each having 150 mg in weight.

Separately, a coating solution is prepared by suspending 6.9 g of hydroxypropylmethyl-cellulose 2910, 1.2 g of polyethylene glycol 6000, 3.3 g of titanium dioxide and 2.1 g of purified talc in 72.6 g of water. Using a High Coated, the 3,000 tablets prepared above are coated with the coating solution to give film-coated tablets, each having 154.5 mg in weight.

### Composition Example 3

Preparation of capsules
Formulation:

| | |
|---|---|
| Compound of the present invention | 5.0 mg |
| Lactose monohydrate | 200 mg |
| Colloidal silicon dioxide | 2 mg |
| Corn starch | 20 mg |
| Magnesium stearate | 4 mg |

25 g of active compound, 1 Kg of lactose monohydrate, 10 g of colloidal silicon dioxide, 100 g of corn starch and 20 g of magnesium stearate are mixed. The mixture is sieved through a 60 mesh sieve, and then filled into 5,000 gelatine capsules.

### Composition Example 4

Preparation of a cream
Formulation:

| | |
|---|---|
| Compound of the present invention | 1% |
| Cetyl alcohol | 3% |
| Stearyl alcohol | 4% |
| Gliceryl monostearate | 4% |
| Sorbitan monostearate | 0.8% |
| Sorbitan monostearate POE | 0.8% |
| Liquid vaseline | 5% |
| Methylparaben | 0.18% |
| Propylparaben | 0.02% |
| Glycerine | 15% |
| Purified water csp. | 100% |

An oil-in-water emulsion cream is prepared with the ingredients listed above, using conventional methods.

1-18. (canceled)

19. A compound of formula (I) 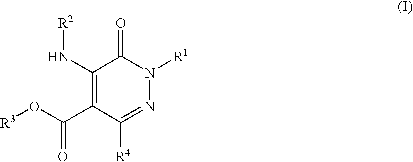
wherein
R¹ represents:
a hydrogen atom;
an alkyl, alkenyl or alkynyl group, which is optionally substituted by one or more substituents selected from halogen atoms and hydroxy, alkoxy, aryloxy, alkylthio, arylthio, oxo, amino, mono-alkylamino, di-alkylamino, acylamino, hydroxycarbonyl, alkoxycarbonyl, carbamoyl, mono-alkylcarbamoyl, and di-alkylcarbamoyl groups;
R² represents a monocyclic or polycyclic heteroaryl group, which is optionally substituted by one or more substituents selected from:
halogen atoms;
alkyl and alkylene groups, which are optionally substituted by one or more substituents selected from halogen atoms and phenyl, hydroxy, alkoxy, aryloxy, alkylthio, arylthio, oxo, amino, mono-alkylamino, di-alkylamino, acylamino, hydroxycarbonyl, alkoxycarbonyl, carbamoyl, mono-alkylcarbamoyl, and di-alkylcarbamoyl groups; and
phenyl, hydroxy, hydroxycarbonyl, hydroxyalkyl, alkoxycarbonyl, alkoxy, cycloalkoxy, nitro, cyano, aryloxy, alkylthio, arylthio, alkylsulfinyl, alkylsulfonyl, alkylsulfamoyl, acyl, amino, mono- or di-alkylamino, acylamino, hydroxycarbonyl, alkoxycarbonyl, carbamoyl, mono-alkylcarbamoyl, di-alkylcarbamoyl, ureido, N′-alkylureido, N′,N′-dialkylureido, alkylsulfamido, aminosulfonyl, mono-alkylaminosulfonyl, di-alkylaminosulfonyl, cyano, difluoromethoxy, and trifluoromethoxy groups;
R³ represents a group of formula:
G-L1-(CRR′)ₙ—
wherein
n is an integer from 0 to 6;
R and R′ are each independently selected from hydrogen atoms or lower alkyl groups;
L1 is a linker selected from a direct bond or —CO—, —NR″—, —NR″—CO—, —O(CO)NR″—, —NR″(CO)O—, —O(CO)—, —O(CO)O—, —(CO)O— or —O(R″O)(PO)O— group wherein R″ is selected from hydrogen atoms or lower alkyl groups; and
G is selected from hydrogen atoms or alkyl, alkenyl, alkynyl, cycloalkyl, cycloalkenyl, heterocyclyl, aryl, arylalkyl, or heteroaryl groups said groups being optionally substituted with one or more substituents selected from:
halogen atoms;
alkyl and alkenyl groups, which are optionally substituted by one or more substituents selected from halogen atoms; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono-alkylaminosulfonyl, di-alkylaminosulfonyl, cyano, difluoromethoxy, and trifluoromethoxy groups;
with the proviso that R³ is not a hydrogen atom; and
R⁴ represents a monocyclic or polycyclic aryl group or monocyclic or polycyclic heteroaryl group, which is optionally substituted by one or more substituents selected from:
halogen atoms;
alkyl and alkenyl groups, which are optionally substituted by one or more substituents selected from halogen atoms and phenyl, hydroxy, hydroxyalkyl, alkoxy, aryloxy, alkylthio, arylthio, oxo, amino, mono-alkylamino, di-alkylamino, acylamino, hydroxycarbonyl, alkoxycarbonyl, carbamoyl, mono-alkylcarbamoyl, and di-alkylcarbamoyl groups; and
hydroxy, alkylenedioxy, alkoxy, cycloalkyloxy, alkylthio, alkylsulfinyl, alkylsulfonyl, alkylsulfamoyl, amino, mono-alkylamino, di-alkylamino, acylamino, nitro, acyl, hydroxycarbonyl, alkoxycarbonyl, carbamoyl, mono-alkylcarbamoyl, di-alkylcarbamoyl, ureido, N′-alkylureido, N′,N′-dialkylureido, alkylsulfamido, aminosulfonyl, mono-alkylaminosulfonyl, di-alkylaminosulfonyl, cyano, difluoromethoxy, and trifluoromethoxy groups;
or a pharmaceutically acceptable salts or N-oxides thereof.

20. A compound according to claim 19 wherein R¹ is a hydrogen atom or lower alkyl group, which is optionally substituted by one or more substituents selected from halogen atoms and hydroxy, alkoxy, alkylthio, hydroxycarbonyl and alkoxycarbonyl groups.

21. A compound according to claim 19 wherein R² is an heteroaryl group which is optionally substituted by one or more substituents selected from halogen atoms and hydroxy, lower alkyl, hydroxyalkyl, hydroxycarbonyl, alkoxy, alkylenedioxy, alkoxycarbonyl, aryloxy, acyl, acyloxy, alkylthio, arylthio, amino, nitro, cyano, mono-alkylamino, di-alkylamino, acylamino, carbamoyl, mono-alkylcarbamoyl, di-alkylcarbamoyl, difluoromethyl, trifluoromethyl, difluoromethoxy, and trifluoromethoxy groups.

22. A compound according to claim 19 wherein R² is a N-containing heteroaryl group.

23. A compound according to claim 19 wherein R² is optionally substituted by one or more substituents selected from halogen atoms and lower alkyl groups.

24. A compound according to claim 19 wherein R³ represents a group of formula:
G-L1-(CRR′)ₙ—
wherein
n is an integer from 0 to 3;
R and R′ are independently selected from hydrogen atoms or lower alkyl groups;
L1 is a linker selected from a direct bond or —CO—, —O(CO)—, —O(CO)O— or —(CO)O— groups; and
G is selected from hydrogen atoms or alkyl, cycloalkyl, heterocyclyl, aryl or heteroaryl groups said groups being optionally substituted with one or more substituents selected from:
halogen atoms;
alkyl and alkenyl groups, which are optionally substituted by one or more substituents selected from halogen atoms; and
hydroxy, alkylenedioxy, alkoxy, cycloalkyloxy, alkylthio, alkylsulfinyl, alkylsulfonyl, alkylsulfamoyl, amino, mono-alkylamino, di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono-alkylaminosulfonyl, di-alkylaminosulfonyl, cyano, difluoromethoxy, and trifluoromethoxy groups.

25. A compound according to claim 24 wherein R³ represents a group of formula:
G-L1-(CRR′)ₙ—
wherein
n is an integer from 0 to 3;
R and R′ are independently selected from hydrogen atoms or methyl groups;
L1 is a linker selected from a direct bond or —CO—, —O(CO)—, —O(CO)O— or —(CO)O— groups; and
G is selected from alkyl, cycloalkyl, heterocyclyl, aryl or heteroaryl groups said groups being optionally substituted with one or more halogen atoms.

26. A compound according to claim 25 wherein R³ represents a group of formula:
G-L1-(CRR′)ₙ—
wherein
n is 0 or 1
R is a hydrogen atom
R′ is a hydrogen atom or a methyl group
L1 is a linker selected from a direct bond —O(CO)O— or —(CO)O—; and
G is selected from alkyl or cycloalkyl groups said groups being optionally substituted with one halogen atom.

27. A compound according to claim 19 wherein R⁴ represents a phenyl, pyridyl, or thienyl group, which is optionally substituted by one or more substituents selected from:
halogen atoms;
alkyl groups, which are optionally substituted by one or more substituents selected from halogen atoms and hydroxy, hydroxyalkyl, alkoxy, alkylthio, mono-alkylamino, di-alkylamino, acylamino, hydroxycarbonyl, alkoxycarbonyl, carbamoyl, mono-alkylcarbamoyl, and di-alkylcarbamoyl groups; and
hydroxy, alkylenedioxy, alkoxy, cycloalkyloxy, alkylthio, alkylsulfinyl, alkylsulfonyl, alkylsulfamoyl, amino, mono- or di-alkylamino, acylamino, nitro, acyl, hydroxycarbonyl, alkoxycarbonyl, carbamoyl, mono- or di-alkylcarbamoyl, ureido, N′-alkylureido, N′,N′-dialkylureido, alkylsulfamido, aminosulfonyl, mono-alkylaminosulfonyl, di-alkylaminosulfonyl, cyano, difluoromethoxy, and trifluoromethoxy groups.

28. A compound according to claim 27 wherein R⁴ is optionally substituted by one or more substituents selected from halogen atoms and lower alkyl groups.

29. A compound according to claim 28 wherein R⁴ is a phenyl group.

30. A compound according to claim 19 which is one of:
4-(methoxycarbonyl)benzyl 1-ethyl-6-oxo-3-phenyl-5-(quinolin-5-ylamino)-1,6-dihydropyridazine-4-carboxylate;
benzyl 1-ethyl-6-oxo-3-phenyl-5-(quinolin-5-ylamino)-1,6-dihydropyridazine-4-carboxylate;
2-(benzyloxy)-2-oxoethyl 1-ethyl-6-oxo-3-phenyl-5-(quinolin-5-ylamino)-1,6-dihydropyridazine-4-carboxylate;
2-ethoxy-2-oxoethyl 1-ethyl-6-oxo-3-phenyl-5-(quinolin-5-ylamino)-1,6-dihydropyridazine-4-carboxylate;
2-oxo-2-pyrrolidin-1-ylethyl 1-ethyl-6-oxo-3-phenyl-5-(quinolin-5-ylamino)-1,6-dihydropyridazine-4-carboxylate;
3-amino-3-oxopropyl 1-ethyl-6-oxo-3-phenyl-5-(quinolin-5-ylamino)-1,6-dihydropyridazine-4-carboxylate;
2-(dimethylamino)ethyl 1-ethyl-6-oxo-3-phenyl-5-(quinolin-5-ylamino)-1,6-dihydropyridazine-4-carboxylate;
2-[(tert-butoxycarbonyl)amino]ethyl 1-ethyl-6-oxo-3-phenyl-5-(quinolin-5-ylamino)-1,6-dihydropyridazine-4-carboxylate;
2-(acetyloxy)ethyl 1-ethyl-6-oxo-3-phenyl-5-(quinolin-5-ylamino)-1,6-dihydropyridazine-4-carboxylate;
3-fluorobenzyl 1-ethyl-6-oxo-3-phenyl-5-(quinolin-5-ylamino)-1,6-dihydropyridazine-4-carboxylate;
[(2,2-dimethylpropanoyl)oxy]methyl 1-ethyl-6-oxo-3-phenyl-5-(quinolin-5-ylamino)-1,6-dihydropyridazine-4-carboxylate;
2-oxo-2-pyridin-4-ylethyl 1-ethyl-6-oxo-3-phenyl-5-(quinolin-5-ylamino)-1,6-dihydropyridazine-4-carboxylate;
2-(dimethylamino)-2-oxoethyl 1-ethyl-6-oxo-3-phenyl-5-(quinolin-5-ylamino)-1,6-dihydropyridazine-4-carboxylate;
2-aminoethyl 1-ethyl-6-oxo-3-phenyl-5-(quinolin-5-ylamino)-1,6-dihydropyridazine-4-carboxylate;
ethyl 1-ethyl-6-oxo-3-phenyl-5-(pyridin-3-ylamino)-1,6-dihydropyridazine-4-carboxylate;
2-(benzyloxy)-2-oxoethyl 1-ethyl-6-oxo-3-phenyl-5-(pyridin-3-ylamino)-1,6-dihydropyridazine-4-carboxylate;
(butyryloxy)methyl 1-ethyl-6-oxo-3-phenyl-5-(pyridin-3-ylamino)-1,6-dihydropyridazine-4-carboxylate;
3-oxo-1,3-dihydro-2-benzofuran-1-yl 1-ethyl-6-oxo-3-phenyl-5-(pyridin-3-ylamino)-1,6-dihydropyridazine-4-carboxylate;
(acetyloxy)methyl 1-ethyl-6-oxo-3-phenyl-5-(pyridin-3-ylamino)-1,6-dihydropyridazine-4-carboxylate;
1-(acetyloxy)ethyl 1-ethyl-6-oxo-3-phenyl-5-(pyridin-3-ylamino)-1,6-dihydropyridazine-4-carboxylate;
2-(dimethylamino)-2-oxoethyl 1-ethyl-6-oxo-3-phenyl-5-(pyridin-3-ylamino)-1,6-dihydropyridazine-4-carboxylate;
benzyl 1-ethyl-6-oxo-3-phenyl-5-(pyridin-3-ylamino)-1,6-dihydropyridazine-4-carboxylate;
[(2,2-dimethylpropanoyl)oxy]methyl 1-ethyl-6-oxo-3-phenyl-5-(pyridin-3-ylamino)-1,6-dihydropyridazine-4-carboxylate;
1-(acetyloxy)-1-methylethyl 1-ethyl-6-oxo-3-phenyl-5-(pyridin-3-ylamino)-1,6-dihydropyridazine-4-carboxylate;
ethyl 1-ethyl-5-[(4-methylpyridin-3-yl)amino]-6-oxo-3-phenyl-1,6-dihydropyridazine-4-carboxylate;
[(2,2-dimethylpropanoyl)oxy]methyl 1-ethyl-5-[(4-methylpyridin-3-yl)amino]-6-oxo-3-phenyl-1,6-dihydropyridazine-4-carboxylate;
1-[(ethoxycarbonyl)oxy]ethyl 1-ethyl-5-[(4-methylpyridin-3-yl)amino]-6-oxo-3-phenyl-1,6-dihydropyridazine-4-carboxylate;
2-(benzyloxy)-2-oxoethyl 1-ethyl-5-[(4-methylpyridin-3-yl)amino]-6-oxo-3-phenyl-1,6-dihydropyridazine-4-carboxylate;
1-[(ethoxycarbonyl)oxy]ethyl 1-ethyl-6-oxo-3-phenyl-5-(pyridin-3-ylamino)-1,6-dihydropyridazine-4-carboxylate;
ethyl 1-ethyl-5-(isoquinolin-4-ylamino)-6-oxo-3-phenyl-1,6-dihydropyridazine-4-carboxylate;
[(2,2-dimethylpropanoyl)oxy]methyl 1-ethyl-5-(isoquinolin-4-ylamino)-6-oxo-3-phenyl-1,6-dihydropyridazine-4-carboxylate;
1-(acetyloxy)ethyl 1-ethyl-5-(isoquinolin-4-ylamino)-6-oxo-3-phenyl-1,6-dihydropyridazine-4-carboxylate;
({[1-ethyl-6-oxo-3-phenyl-5-(pyridin-3-ylamino)-1,6-dihydropyridazin-4-yl]carbonyl}oxy)acetic acid;
ethyl 1-ethyl-3-(3-methylphenyl)-6-oxo-5-(pyridin-3-ylamino)-1,6-dihydropyridazine-4-carboxylate;
[(2,2-dimethylpropanoyl)oxy]methyl 1-ethyl-3-(3-methylphenyl)-6-oxo-5-(pyridin-3-ylamino)-1,6-dihydropyridazine-4-carboxylate;
ethyl 1-ethyl-3-(3-fluorophenyl)-6-oxo-5-(pyridin-3-ylamino)-1,6-dihydropyridazine-4-carboxylate;
(butyryloxy)methyl 1-ethyl-3-(3-fluorophenyl)-6-oxo-5-(pyridin-3-ylamino)-1,6-dihydropyridazine-4-carboxylate;
ethyl 1-ethyl-3-(4-fluorophenyl)-6-oxo-5-(pyridin-3-ylamino)-1,6-dihydropyridazine-4-carboxylate;
(butyryloxy)methyl 1-ethyl-3-(4-fluorophenyl)-6-oxo-5-(pyridin-3-ylamino)-1,6-dihydropyridazine-4-carboxylate;
ethyl 5-[(2-chloropyridin-3-yl)amino]-1-ethyl-6-oxo-3-phenyl-1,6-dihydropyridazine-4-carboxylate;
methyl 1-ethyl-6-oxo-3-phenyl-5-(pyridin-3-ylamino)-1,6-dihydropyridazine-4-carboxylate;
methyl 1-ethyl-6-oxo-3-phenyl-5-(quinolin-5-ylamino)-1,6-dihydropyridazine-4-carboxylate;
ethyl 1-ethyl-6-oxo-5-(pyridin-3-ylamino)-3-(2-thienyl)-1,6-dihydropyridazine-4-carboxylate;
2-(acetyloxy)ethyl 1-ethyl-6-oxo-5-(pyridin-3-ylamino)-3-(2-thienyl)-1,6-dihydropyridazine-4-carboxylate;
2-[(tert-butoxycarbonyl)amino]ethyl 1-ethyl-6-oxo-5-(pyridin-3-ylamino)-3-(2-thienyl)-1,6-dihydropyridazine-4-carboxylate;
2-ethoxy-2-oxoethyl 1-ethyl-6-oxo-5-(pyridin-3-ylamino)-3-(2-thienyl)-1,6-dihydropyridazine-4-carboxylate;
2-(benzyloxy)-2-oxoethyl 1-ethyl-6-oxo-5-(pyridin-3-ylamino)-3-(2-thienyl)-1,6-dihydropyridazine-4-carboxylate;
benzyl 1-ethyl-6-oxo-5-(pyridin-3-ylamino)-3-(2-thienyl)-1,6-dihydropyridazine-4-carboxylate;
ethyl 1-ethyl-5-(4-methyloyridin-3-ylamino)-6-oxo-3-thien-2-yl-1,6-dihydropyridazin-4-carboxylate;
2-(acetyloxy)ethyl 1-ethyl-5-[(4-methylpyridin-3-yl)amino]-6-oxo-3-(2-thienyl)-1,6-dihydropyridazine-4-carboxylate;
2-[(tert-butoxycarbonyl)amino]ethyl 1-ethyl-5-[(4-methylpyridin-3-yl)amino]-6-oxo-3-(2-thienyl)-1,6-dihydropyridazine-4-carboxylate;
ethyl 1-ethyl-5-(isoquinolin-4-ylamino)-6-oxo-3-(2-thienyl)-1,6-dihydropyridazine-4-carboxylate;
2-(acetyloxy)ethyl 1-ethyl-5-(isoquinolin-4-ylamino)-6-oxo-3-(2-thienyl)-1,6-dihydropyridazine-4-carboxylate;
2-[(tert-butoxycarbonyl)amino]ethyl 1-ethyl-5-(isoquinolin-4-ylamino)-6-oxo-3-(2-thienyl)-1,6-dihydropyridazine-4-carboxylate;
2-ethoxy-2-oxoethyl 1-ethyl-5-(isoquinolin-4-ylamino)-6-oxo-3-(2-thienyl)-1,6-dihydropyridazine-4-carboxylate;
2-(benzyloxy)-2-oxoethyl 1-ethyl-5-(isoquinolin-4-ylamino)-6-oxo-3-(2-thienyl)-1,6-dihydropyridazine-4-carboxylate;
benzyl 1-ethyl-5-(isoquinolin-4-ylamino)-6-oxo-3-(2-thienyl)-1,6-dihydropyridazine-4-carboxylate;
ethyl 1-ethyl-5-(isoquinolin-4-ylamino)-6-oxo-3-(3-thienyl)-1,6-dihydropyridazine-4-carboxylate;
2-(acetyloxy)ethyl 1-ethyl-5-(isoquinolin-4-ylamino)-6-oxo-3-(3-thienyl)-1,6-dihydropyridazine-4-carboxylate;
2-[(tert-butoxycarbonyl)amino]ethyl 1-ethyl-5-(isoquinolin-4-ylamino)-6-oxo-3-(3-thienyl)-1,6-dihydropyridazine-4-carboxylate;
2-ethoxy-2-oxoethyl 1-ethyl-5-(isoquinolin-4-ylamino)-6-oxo-3-(3-thienyl)-1,6-dihydropyridazine-4-carboxylate;
2-(benzyloxy)-2-oxoethyl 1-ethyl-5-(isoquinolin-4-ylamino)-6-oxo-3-(3-thienyl)-1,6-dihydropyridazine-4-carboxylate;
4-fluorobenzyl 1-ethyl-5-(isoquinolin-4-ylamino)-6-oxo-3-(2-thienyl)-1,6-dihydropyridazine-4-carboxylate;
4-(methoxycarbonyl)benzyl 1-ethyl-5-(isoquinolin-4-ylamino)-6-oxo-3-(2-thienyl)-1,6-dihydropyridazine-4-carboxylate;
ethyl 1-ethyl-3-(4-methylphenyl)-6-oxo-5-(pyridin-3-ylamino)-1,6-dihydropyridazine-4-carboxylate;
ethyl 1-ethyl-5-(isoquinolin-4-ylamino)-3-(4-methylphenyl)-6-oxo-1,6-dihydropyridazine-4-carboxylate;
ethyl 1-ethyl-3-(4-methylphenyl)-5-[(4-methylpyridin-3-yl)amino]-6-oxo-1,6-dihydropyridazine-4-carboxylate;
[(2,2-dimethylpropanoyl)oxy]methyl 1-ethyl-3-(4-methylphenyl)-6-oxo-5-(pyridin-3-ylamino)-1,6-dihydropyridazine-4-carboxylate;
[(2,2-dimethylpropanoyl)oxy]methyl 1-ethyl-5-(isoquinolin-4-ylamino)-3-(4-methylphenyl)-6-oxo-1,6-dihydropyridazine-4-carboxylate;
[(2,2-dimethylpropanoyl)oxy]methyl 1-ethyl-3-(4-methylphenyl)-5-[(4-methylpyridin-3-yl)amino]-6-oxo-1,6-dihydropyridazine-4-carboxylate;
1-[(isopropoxycarbonyl)oxy]ethyl 1-ethyl-6-oxo-3-phenyl-5-(pyridin-3-ylamino)-1,6-dihydropyridazine-4-carboxylate;
1-[(isopropoxycarbonyl)oxy]ethyl 1-ethyl-5-(isoquinolin-4-ylamino)-6-oxo-3-phenyl-1,6-dihydropyridazine-4-carboxylate;
1-[(isopropoxycarbonyl)oxy]ethyl 1-ethyl-5-[(4-methylpyridin-3-yl)amino]-6-oxo-3-phenyl-1,6-dihydropyridazine-4-carboxylate;
1-{[(cyclohexyloxy)carbonyl]oxy}ethyl 1-ethyl-6-oxo-3-phenyl-5-(pyridin-3-ylamino)-1,6-dihydropyridazine-4-carboxylate;
1-{[(cyclohexyloxy)carbonyl]oxy}ethyl 1-ethyl-5-(isoquinolin-4-ylamino)-6-oxo-3-phenyl-1,6-dihydropyridazine-4-carboxylate;
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate;
ethyl 1-ethyl-6-oxo-3-phenyl-5-(thieno[2,3-c]pyridin-3-ylamino)-1,6-dihydropyridazine-4-carboxylate;
ethyl 1-ethyl-6-oxo-3-phenyl-5-(thieno[2,3-b]pyridin-3-ylamino)-1,6-dihydropyridazine-4-carboxylate;
[(2,2-dimethylpropanoyl)oxy]methyl 1-ethyl-6-oxo-3-phenyl-5-(thieno[2,3-b]pyridin-3-ylamino)-1,6-dihydropyridazine-4-carboxylate;
7-ethoxy-7-oxoheptyl 1-ethyl-5-(isoquinolin-4-ylamino)-6-oxo-3-(3-thienyl)-1,6-dihydropyridazine-4-carboxylate;
6-ethoxy-6-oxohexyl 1-ethyl-5-(isoquinolin-4-ylamino)-6-oxo-3-(3-thienyl)-1,6-dihydropyridazine-4-carboxylate;
3-amino-3-oxopropyl 1-ethyl-5-(isoquinolin-4-ylamino)-6-oxo-3-(3-thienyl)-1,6-dihydropyridazine-4-carboxylate;
2-ethoxy-2-oxoethyl 1-ethyl-5-[(4-methylpyridin-3-yl)amino]-6-oxo-3-(2-thienyl)-1,6-dihydropyridazine-4-carboxylate;
2-(benzyloxy)-2-oxoethyl 1-ethyl-5-[(4-methylpyridin-3-yl)amino]-6-oxo-3-(2-thienyl)-1,6-dihydropyridazine-4-carboxylate;
1-{[(1-ethylpropoxy)carbonyl]oxy}ethyl 1-ethyl-6-oxo-3-phenyl-5-(pyridin-3-ylamino)-1,6-dihydropyridazine-4-carboxylate;
1-{[(1-ethylpropoxy)carbonyl]oxy}ethyl 1-ethyl-5-[(4-methylpyridin-3-yl)amino]-6-oxo-3-phenyl-1,6-dihydropyridazine-4-carboxylate;
1-{[(1-ethylpropoxy)carbonyl]oxy}ethyl 1-ethyl-5-(isoquinolin-4-ylamino)-6-oxo-3-phenyl-1,6-dihydropyridazine-4-carboxylate;
(butyryloxy)methyl 1-ethyl-5-(isoquinolin-4-ylamino)-6-oxo-3-phenyl-1,6-dihydropyridazine-4-carboxylate;
(acetyloxy)methyl 1-ethyl-5-[(4-methylpyridin-3-yl)amino]-6-oxo-3-phenyl-1,6-dihydropyridazine-4-carboxylate;
benzyl 1-ethyl-5-[(4-methylpyridin-3-yl)amino]-6-oxo-3-(2-thienyl)-1,6-dihydropyridazine-4-carboxylate;
4-(methoxycarbonyl)benzyl 1-ethyl-5-[(4-methylpyridin-3-yl)amino]-6-oxo-3-(2-thienyl)-1,6-dihydropyridazine-4-carboxylate;
(isobutyryloxy)methyl 1-ethyl-5-(isoquinolin-4-ylamino)-6-oxo-3-phenyl-1,6-dihydropyridazine-4-carboxylate;
(isobutyryloxy)methyl 1-ethyl-6-oxo-3-phenyl-5-(pyridin-3-ylamino)-1,6-dihydropyridazine-4-carboxylate;
4-fluorobenzyl 1-ethyl-6-oxo-5-(pyridin-3-ylamino)-3-(2-thienyl)-1,6-dihydropyridazine-4-carboxylate;
4-(methoxycarbonyl)benzyl 1-ethyl-6-oxo-5-(pyridin-3-ylamino)-3-(2-thienyl)-1,6-dihydropyridazine-4-carboxylate;
[(isopropoxycarbonyl)oxy]methyl 1-ethyl-6-oxo-3-phenyl-5-(pyridin-3-ylamino)-1,6-dihydropyridazine-4-carboxylate;
7-ethoxy-7-oxoheptyl 1-ethyl-6-oxo-5-(pyridin-3-ylamino)-3-(2-thienyl)-1,6-dihydropyridazine-4-carboxylate;
6-ethoxy-6-oxohexyl 1-ethyl-6-oxo-5-(pyridin-3-ylamino)-3-(2-thienyl)-1,6-dihydropyridazine-4-carboxylate;
4-fluorobenzyl 1-ethyl-5-[(4-methylpyridin-3-yl)amino]-6-oxo-3-(2-thienyl)-1,6-dihydropyridazine-4-carboxylate;
(5-methyl-2-oxo-1,3-dioxol-4-yl)methyl 1-ethyl-6-oxo-3-phenyl-5-(pyridin-3-ylamino)-1,6-dihydropyridazine-4-carboxylate;
ethyl 1-ethyl-5-[(4-methylpyridin-3-yl)amino]-6-oxo-3-(3-thienyl)-1,6-dihydropyridazine-4-carboxylate;
chloromethyl 1-ethyl-6-oxo-3-phenyl-5-(pyridin-3-ylamino)-1,6-dihydropyridazine-4-carboxylate;
{[(cyclohexyloxy)carbonyl]oxy}methyl 1-ethyl-5-[(4-methylpyridin-3-yl)amino]-6-oxo-3-phenyl-1,6-dihydropyridazine-4-carboxylate;
(5-methyl-2-oxo-1,3-dioxol-4-yl)methyl 1-ethyl-5-[(4-methylpyridin-3-yl)amino]-6-oxo-3-phenyl-1,6-dihydropyridazine-4-carboxylate;
[(2,2-dimethylbutanoyl)oxy]methyl 1-ethyl-6-oxo-3-phenyl-5-(pyridin-3-ylamino)-1,6-dihydropyridazine-4-carboxylate;
({N-[(benzyloxy)carbonyl]-L-valyl}oxy)methyl 1-ethyl-6-oxo-3-phenyl-5-(pyridin-3-ylamino)-1,6-dihydropyridazine-4-carboxylate;
(5-methyl-2-oxo-1,3-dioxol-4-yl)methyl 1-ethyl-5-(isoquinolin-4-ylamino)-6-oxo-3-phenyl-1,6-dihydropyridazine-4-carboxylate;
ethyl 1-ethyl-6-oxo-5-(pyridin-3-ylamino)-3-(3-thienyl)-1,6-dihydropyridazine-4-carboxylate;
{[(cyclohexyloxy)carbonyl]oxy}methyl 1-ethyl-5-(isoquinolin-4-ylamino)-6-oxo-3-phenyl-1,6-dihydropyridazine-4-carboxylate;
{[(cyclohexyloxy)carbonyl]oxy}methyl 1-ethyl-6-oxo-3-phenyl-5-(pyridin-3-ylamino)-1,6-dihydropyridazine-4-carboxylate;
2-(acetyloxy)ethyl 1-ethyl-5-[(4-methylpyridin-3-yl)amino]-6-oxo-3-(3-thienyl)-1,6-dihydropyridazine-4-carboxylate;
2-[(tert-butoxycarbonyl)amino]ethyl 1-ethyl-5-[(4-methylpyridin-3-yl)amino]-6-oxo-3-(3-thienyl)-1,6-dihydropyridazine-4-carboxylate;
{[(1-ethylpropoxy)carbonyl]oxy}methyl 1-ethyl-6-oxo-3-phenyl-5-(pyridin-3-ylamino)-1,6-dihydropyridazine-4-carboxylate;
(isobutyryloxy)methyl 1-ethyl-5-[(4-methylpyridin-3-yl)amino]-6-oxo-3-phenyl-1,6-dihydropyridazine-4-carboxylate;
2-(acetyloxy)ethyl 1-ethyl-6-oxo-5-(pyridin-3-ylamino)-3-(3-thienyl)-1,6-dihydropyridazine-4-carboxylate;
2-[(tert-butoxycarbonyl)amino]ethyl 1-ethyl-6-oxo-5-(pyridin-3-ylamino)-3-(3-thienyl)-1,6-dihydropyridazine-4-carboxylate;
2-ethoxy-2-oxoethyl 1-ethyl-6-oxo-5-(pyridin-3-ylamino)-3-(3-thienyl)-1,6-dihydropyridazine-4-carboxylate;
[(isopropoxycarbonyl)oxy]methyl 1-ethyl-5-(isoquinolin-4-ylamino)-6-oxo-3-phenyl-1,6-dihydropyridazine-4-carboxylate;
2-(benzyloxy)-2-oxoethyl 1-ethyl-6-oxo-5-(pyridin-3-ylamino)-3-(3-thienyl)-1,6-dihydropyridazine-4-carboxylate;
2-ethoxy-2-oxoethyl 1-ethyl-5-[(4-methylpyridin-3-yl)amino]-6-oxo-3-(3-thienyl)-1,6-dihydropyridazine-4-carboxylate;
2-(benzyloxy)-2-oxoethyl 1-ethyl-5-[(4-methylpyridin-3-yl)amino]-6-oxo-3-(3-thienyl)-1,6-dihydropyridazine-4-carboxylate;
{[(1-ethylpropoxy)carbonyl]oxy}methyl 1-ethyl-5-(isoquinolin-4-ylamino)-6-oxo-3-phenyl-1,6-dihydropyridazine-4-carboxylate;
benzyl 1-ethyl-5-[(4-methylpyridin-3-yl)amino]-6-oxo-3-(3-thienyl)-1,6-dihydropyridazine-4-carboxylate;
benzyl 1-ethyl-6-oxo-5-(pyridin-3-ylamino)-3-(3-thienyl)-1,6-dihydropyridazine-4-carboxylate;
[(isopropoxycarbonyl)oxy]methyl 1-ethyl-5-[(4-methylpyridin-3-yl)amino]-6-oxo-3-phenyl-1,6-dihydropyridazine-4-carboxylate;
6-ethoxy-6-oxohexyl 1-ethyl-5-(isoquinolin-4-ylamino)-6-oxo-3-(2-thienyl)-1,6-dihydropyridazine-4-carboxylate;
7-ethoxy-7-oxoheptyl 1-ethyl-5-(isoquinolin-4-ylamino)-6-oxo-3-(2-thienyl)-1,6-dihydropyridazine-4-carboxylate;
({[1-ethyl-6-oxo-3-phenyl-5-(pyridin-3-ylamino)-1,6-dihydropyridazin-4-yl]carbonyl}oxy)methyl N-(tert-butoxycarbonyl)-L-leucinate;
2-methoxy-2-oxoethyl 1-ethyl-5-(isoquinolin-4-ylamino)-6-oxo-3-phenyl-1,6-dihydropyridazine-4-carboxylate;
4-fluorobenzyl 1-ethyl-6-oxo-5-(pyridin-3-ylamino)-3-(3-thienyl)-1,6-dihydropyridazine-4-carboxylate;
4-(methoxycarbonyl)benzyl 1-ethyl-6-oxo-5-(pyridin-3-ylamino)-3-(3-thienyl)-1,6-dihydropyridazine-4-carboxylate;
(butyryloxy)methyl 1-ethyl-5-[(4-methylpyridin-3-yl)amino]-6-oxo-3-phenyl-1,6-dihydropyridazine-4-carboxylate;
6-ethoxy-6-oxohexyl 1-ethyl-5-[(4-methylpyridin-3-yl)amino]-6-oxo-3-(2-thienyl)-1,6-dihydropyridazine-4-carboxylate;
{[(1-ethylpropoxy)carbonyl]oxy}methyl 1-ethyl-5-[(4-methylpyridin-3-yl)amino]-6-oxo-3-phenyl-1,6-dihydropyridazine-4-carboxylate;
7-ethoxy-7-oxoheptyl 1-ethyl-5-[(4-methylpyridin-3-yl)amino]-6-oxo-3-(2-thienyl)-1,6-dihydropyridazine-4-carboxylate;
({[1-ethyl-6-oxo-3-phenyl-5-(pyridin-3-ylamino)-1,6-dihydropyridazin-4-yl]carbonyl}oxy)methyl L-leucinate;
benzyl 1-ethyl-5-(isoquinolin-4-ylamino)-6-oxo-3-(3-thienyl)-1,6-dihydropyridazine-4-carboxylate;
3-amino-3-oxopropyl 1-ethyl-5-[(4-methylpyridin-3-yl)amino]-6-oxo-3-(3-thienyl)-1,6-dihydropyridazine-4-carboxylate;
4-fluorobenzyl 1-ethyl-5-[(4-methylpyridin-3-yl)amino]-6-oxo-3-(3-thienyl)-1,6-dihydropyridazine-4-carboxylate;
4-(methoxycarbonyl)benzyl 1-ethyl-5-[(4-methylpyridin-3-yl)amino]-6-oxo-3-(3-thienyl)-1,6-dihydropyridazine-4-carboxylate;
[(2-methylbutanoyl)oxy]methyl 1-ethyl-6-oxo-3-phenyl-5-(pyridin-3-ylamino)-1,6-dihydropyridazine-4-carboxylate;
4-(methoxycarbonyl)benzyl 1-ethyl-5-(isoquinolin-4-ylamino)-6-oxo-3-(3-thienyl)-1,6-dihydropyridazine-4-carboxylate;
7-ethoxy-7-oxoheptyl 1-ethyl-5-[(4-methylpyridin-3-yl)amino]-6-oxo-3-(3-thienyl)-1,6-dihydropyridazine-4-carboxylate;
7-ethoxy-7-oxoheptyl 1-ethyl-6-oxo-5-(pyridin-3-ylamino)-3-(3-thienyl)-1,6-dihydropyridazine-4-carboxylate;
4-fluorobenzyl 1-ethyl-5-(isoquinolin-4-ylamino)-6-oxo-3-(3-thienyl)-1,6-dihydropyridazine-4-carboxylate;
6-ethoxy-6-oxohexyl 1-ethyl-5-[(4-methylpyridin-3-yl)amino]-6-oxo-3-(3-thienyl)-1,6-dihydropyridazine-4-carboxylate;
6-ethoxy-6-oxohexyl 1-ethyl-6-oxo-5-(pyridin-3-ylamino)-3-(3-thienyl)-1,6-dihydropyridazine-4-carboxylate;
1-{[(cyclohexyloxy)carbonyl]oxy}ethyl 1-ethyl-5-(1,7-naphthyridin-5-ylamino)-6-oxo-3-phenyl-1,6-dihydropyridazine-4-carboxylate;
benzyl 1-ethyl-6-oxo-3-pyridin-4-yl-5-(pyridin-3-ylamino)-1,6-dihydropyridazine-4-carboxylate;
({[1-ethyl-6-oxo-3-phenyl-5-(pyridin-3-ylamino)-1,6-dihydropyridazin-4-yl]carbonyl}oxy)methyl morpholine-4-carboxylate;
{[(methylamino)carbonyl]oxy}methyl 1-ethyl-6-oxo-3-phenyl-5-(pyridin-3-ylamino)-1,6-dihydropyridazine-4-carboxylate;
{[(dimethylamino)carbonyl]oxy}methyl 1-ethyl-6-oxo-3-phenyl-5-(pyridin-3-ylamino)-1,6-dihydropyridazine-4-carboxylate;
(acetyloxy)methyl 1-ethyl-5-(isoquinolin-4-ylamino)-6-oxo-3-phenyl-1,6-dihydropyridazine-4-carboxylate;
[(dibutoxyphosphoryl)oxy]methyl 1-ethyl-6-oxo-3-phenyl-5-(pyridin-3-ylamino)-1,6-dihydropyridazine-4-carboxylate;
(acetyloxy)methyl 1-ethyl-6-oxo-5-(pyridin-3-ylamino)-3-(2-thienyl)-1,6-dihydropyridazine-4-carboxylate;
[(isopropoxycarbonyl)oxy]methyl 1-ethyl-6-oxo-5-(pyridin-3-ylamino)-3-(2-thienyl)-1,6-dihydropyridazine-4-carboxylate;
{[(cyclohexyloxy)carbonyl]oxy}methyl 1-ethyl-6-oxo-5-(pyridin-3-ylamino)-3-(2-thienyl)-1,6-dihydropyridazine-4-carboxylate;
(acetyloxy)methyl 1-ethyl-5-[(4-methylpyridin-3-yl)amino]-6-oxo-3-(2-thienyl)-1,6-dihydropyridazine-4-carboxylate;
[(isopropoxycarbonyl)oxy]methyl 1-ethyl-5-[(4-methylpyridin-3-yl)amino]-6-oxo-3-(2-thienyl)-1,6-dihydropyridazine-4-carboxylate;
{[(cyclohexyloxy)carbonyl]oxy}methyl 1-ethyl-5-[(4-methylpyridin-3-yl)amino]-6-oxo-3-(2-thienyl)-1,6-dihydropyridazine-4-carboxylate;
(acetyloxy)methyl 1-ethyl-5-(isoquinolin-4-ylamino)-6-oxo-3-(2-thienyl)-1,6-dihydropyridazine-4-carboxylate;
[(isopropoxycarbonyl)oxy]methyl 1-ethyl-5-(isoquinolin-4-ylamino)-6-oxo-3-(2-thienyl)-1,6-dihydropyridazine-4-carboxylate;
{[(cyclohexyloxy)carbonyl]oxy}methyl 1-ethyl-5-(isoquinolin-4-ylamino)-6-oxo-3-(2-thienyl)-1,6-dihydropyridazine-4-carboxylate;
{[(cyclohexyloxy)carbonyl]oxy}methyl 1-ethyl-6-oxo-5-(pyridin-3-ylamino)-3-(3-thienyl)-1,6-dihydropyridazine-4-carboxylate;
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 1;
1-{[(cyclohexyloxy)carbonyl]oxy}ethyl 1-ethyl-5-[(4-methylpyridin-3-yl)amino]-6-oxo-3-phenyl-1,6-dihydropyridazine-4-carboxylate—Enantiomer 2;
chloromethyl 1-ethyl-5-[(4-methylpyridin-3-yl)amino]-6-oxo-3-phenyl-1,6-dihydropyridazine-4-carboxylate;
(propionyloxy)methyl 1-ethyl-5-[(4-methylpyridin-3-yl)amino]-6-oxo-3-phenyl-1,6-dihydropyridazine-4-carboxylate;
{[(1-ethylpropoxy)carbonyl]oxy}methyl 1-ethyl-3-(4-fluorophenyl)-6-oxo-5-(pyridin-3-ylamino)-1,6-dihydropyridazine-4-carboxylate;
{[(cyclohexyloxy)carbonyl]oxy}methyl 1-ethyl-3-(4-fluorophenyl)-6-oxo-5-(pyridin-3-ylamino)-1,6-dihydropyridazine-4-carboxylate;
chloromethyl 1-ethyl-5-(isoquinolin-4-ylamino)-6-oxo-3-phenyl-1,6-dihydropyridazine-4-carboxylate;
(propionyloxy)methyl 1-ethyl-6-oxo-3-phenyl-5-(pyridin-3-ylamino)-1,6-dihydropyridazine-4-carboxylate;
(propionyloxy)methyl 1-ethyl-5-(isoquinolin-4-ylamino)-6-oxo-3-phenyl-1,6-dihydropyridazine-4-carboxylate;
(pentanoyloxy)methyl 1-ethyl-6-oxo-3-phenyl-5-(pyridin-3-ylamino)-1,6-dihydropyridazine-4-carboxylate;
2-oxo-1,3-dioxolan-4-yl 1-ethyl-6-oxo-3-phenyl-5-(pyridin-3-ylamino)-1,6-dihydropyridazine-4-carboxylate;
fluoromethyl 1-ethyl-6-oxo-3-phenyl-5-(pyridin-3-ylamino)-1,6-dihydropyridazine-4-carboxylate;
1-{[(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 1; or
1-{[(cyclohexyloxy)carbonyl]oxy}ethyl 1-ethyl-6-oxo-3-phenyl-5-(pyridin-3-ylamino)-1,6-dihydropyridazine-4-carboxylate—Enantiomer 2;
or a pharmaceutically acceptable salt thereof.

30. A pharmaceutical composition comprising a compound according to claim 19 in admixture with a pharmaceutically acceptable diluent or carrier.

31. A method for treating a subject afflicted with a pathological condition or disease susceptible to amelioration by inhibition of phosphodiesterase 4, which method comprises administering to said subject an effective amount of a compound according to claim 19.

32. A method according to claim 31, wherein the pathological condition or disease is asthma, chronic obstructive pulmonary disease, rheumatoid arthritis, atopic dermatitis, psoriasis or irritable bowel disease.

33. A combination product comprising:
(i) a compound according to claim 19; and
(ii) another compound selected from (a) steroids, (b) immunosuppressive agents, (c) T-cell receptor blockers, (d) antiinflammatory drugs, (e) β2-adrenergic agonists and (f) antagonists of M3 muscarinic receptors
for simultaneous, separate or sequential administration to a human or animal.

34. A method for treating a subject afflicted with a pathological condition or disease susceptible to amelioration by inhibition of phosphodiesterase 4, which method comprises administering to said subject an effective amount of a combination product according to claim 33.
